SEQUENCE LISTING (15 sequences)

SEQ ID NO: 1 - DNA, 381 bp, Artificial Sequence
  Description of Artificial Sequence Synthetic polynucleotide
tcccaagtacaactggtagaaagcggtggtggtctggtacaagcaggtggtagcctgcgtctgagctgcgcagcatcccgtcgttcctcccgttcctgggctatggcttggttccgtcaagcaccaggtaaagaacgtgaatttgtggcgaaaattagcggcgatggccgtctgaccacctatggagacagcgtaaaaggtcgtttcaccatcagccgtgacaacgcggaatatctggtgtatctgcagatggacagcctgaaaccagaagacaccgcagtttactactgcgctgctgatgataactatgtgaccgcgagctggcgtagcggcccggattattggggccagggcacccaagtaaccgtaagcagctaatag

SEQ ID NO: 2 - DNA, 363 bp, Artificial Sequence
  Description of Artificial Sequence Synthetic polynucleotide
tcccaagtacaactggtagaaagcggtggtggtctggtacaagcaggtggtagcctgcgtctgagctgcaccgcttccggtcgtaccggtaccatctactccatggcttggttccgtcaagcaccaggtaaagaacgtgaattcctggctaccgttggttggtcctccggtatcacctactacatggacagcgtaaaaggtcgtttcaccatcagccgtgacaaaggtaaaaacaccgtatacctgcagatggacagcctgaaaccagaagacaccgcagtttactactgcaccgctacccgtgcttactccgttggttacgactactggggtcaaggcacccaagtaaccgtaagcagctaa

SEQ ID NO: 3 - protein, 130 aa, Artificial Sequence
  Description of Artificial Sequence Synthetic polypeptide
MAQVQLVETGGGLVQPGGSLRLSCTASGFTFSMHAMTWYRQAPGKQRELVAVITSHGDRANYTDSVRGRFTISRDNTKNMVYLQMNSLKPEDTAVYYCNVPRYDSWGQGTQVTVSSGGLPETGGHHHHHH

SEQ ID NO: 4 - protein, 129 aa, Artificial Sequence
  Description of Artificial Sequence Synthetic polypeptide
MAQVQLVESGGGLAQPGGSLRLSCAASGSTISSVAVGWYRQTPGNQREWVATSSTSSTTATYADSVKGRFTISRDNAKNTIYLQMNSLKPEDTAVYYCKTGLTNWGRGTQVTVSSGGLPETGGHHHHHH

SEQ ID NO: 5 - protein, 6 aa, Artificial Sequence
  Description of Artificial Sequence Synthetic 6xHis tag
HHHHHH

SEQ ID NO: 6 - DNA, 46 bp, Artificial Sequence
  Description of Artificial Sequence Synthetic primer
tacaaaaacatcagccgtaggatcccatcatcatcatcatcacagc

SEQ ID NO: 7 - DNA, 31 bp, Artificial Sequence
  Description of Artificial Sequence Synthetic primer
gtacttgggatccctggaagtacaggttttc

SEQ ID NO: 8 - DNA, 45 bp, Artificial Sequence
  Description of Artificial Sequence Synthetic primer
cttgaagtcctctttcagggacccagcgtttcggaaattcaattg

SEQ ID NO: 9 - DNA, 47 bp, Artificial Sequence
  Description of Artificial Sequence Synthetic primer
ctgccccggggacgtcgactctagactattagaaattgtgcacatcc

SEQ ID NO: 10 - DNA, 29 bp, Artificial Sequence
  Description of Artificial Sequence Synthetic primer
cttccagggatcccaagtacaactggtag

SEQ ID NO: 11 - DNA, 46 bp, Artificial Sequence
  Description of Artificial Sequence Synthetic primer
ctgccccggggacgtcgactctagactattagctgcttacggttac

SEQ ID NO: 12 - DNA, 42 bp, Artificial Sequence
  Description of Artificial Sequence Synthetic primer
cttgaagtcctctttcagggacccacgccgttgggccccgcc

SEQ ID NO: 13 - DNA, 52 bp, Artificial Sequence
  Description of Artificial Sequence Synthetic primer
ctgccccggggacgtcgactctagactattacggctgtgcaagatgacgcaa

SEQ ID NO: 14 - DNA, 21 bp, Artificial Sequence
  Description of Artificial Sequence Synthetic primer
acaaaaacatcagccgtagga

SEQ ID NO: 15 - DNA, 19 bp, Artificial Sequence
  Description of Artificial Sequence Synthetic primer
ccggggacgtcgactctag

(19) United States
(12) Patent Application Publication
(10) Pub. No.: US 2024/0094220 A1
(43) Pub. Date: Mar. 21, 2024

(54) ENGINEERING BACILLUS SUBTILIS AS A VERSATILE AND STABLE PLATFORM FOR PRODUCTION OF NANOBODIES

(71) Applicant: Northeastern University, Boston, MA (US)

(72) Inventor: Jiahe Li, Medford, MA (US)

(21) Appl. No.: 17/768,094

(22) PCT Filed: Oct. 13, 2020
(86) PCT No.: PCT/US20/55355

§ 371 Date: Apr. 11, 2022

Related U.S. Application Data
(60) Provisional application No. 62/914,130, filed on Oct. 11, 2019.

(51) Int. Cl. G01N 33/68; C07K 16/08; C07K 16/10; C07K 17/12; C12N 1/20; C12N 15/75; G01N 33/532

(52) U.S. Cl. CPC G01N 33/6854; C07K 16/082; C07K 16/1003; C07K 17/12; C12N 1/205; C12N 15/75; G01N 33/532; C07K 2317/22; C07K 2317/569; G01N 2333/02; G01N 2333/165; G01N 2333/32; G01N 2333/4603

(57) ABSTRACT

Provided are methods and pharmaceutical compositions related to nanobodies from engineered Bacillus subtilis bacteria which can be useful as therapeutic agents or for diagnostic testing.

### RELATED APPLICATIONS

This application claims the benefit of priority to U.S. Provisional Patent Application No. 62/914,130, filed Oct. 11, 2019.

### BACKGROUND

There is a growing need for a highly stable system to allow the production of biologics for diagnoses and therapeutic interventions on demand that could be used in extreme environments. Among the variety of biologics, nanobodies (Nbs) derived from single-chain variable antibody fragments from camelids, have attracted great attention in recent years due to their small size and great stability with translational potentials in whole-body imaging and the development of new drugs. Intracellular expression using the bacterium Escherichia coli has been the predominant system to produce Nbs, and this requires lengthy steps for releasing intracellular proteins for purification as well as removal of endotoxins. Lyophilized, translationally competent cell extracts have also been explored as offering portability and long shelf lives, but such extracts may be difficult to scale up and suffer from batch-to-batch variability. Effective, cost-efficient nanobody production and storage systems are currently needed.

### SUMMARY

As disclosed herein, nanobodies obtained from Bacillus subtilis bacteria have therapeutic effects and are useful for the treatment or prevention of disease or health disorders, and are useful for diagnostic testing.

In certain aspects, provided herein is a nanobody comprising a variable domain of an antibody, wherein the nanobody is contained within a Bacillus subtilis bacteria strain. In some embodiments the variable domain is a heavy chain variable domain. In some embodiments, the antibody is a mammalian antibody. In some embodiments, the mammalian antibody is a camelid antibody. In some embodiments, the antibody is a fish antibody.

In some embodiments, the Bacillus subtilis strain is deficient in at least eight extracellular proteases. In some embodiments, the Bacillus subtilis strain are from a strain comprising at least 90% genomic, 16S and/or CRISPR sequence identity to the nucleotide sequence of the Bacillus subtilis strain WB800N (GenBank Accession No. CP032310.1). In some embodiments, the Bacillus subtilis strain are from a strain comprising at least 99% genomic, 16S and/or CRISPR sequence identity to the nucleotide sequence of the Bacillus subtilis strain WB800N (GenBank Accession No. CP032310.1). In some embodiments, the Bacillus subtilis strain are from Bacillus subtilis strain WB800N (GenBank Accession No. CP032310.1).

In some embodiments, the nanobody comprises an affinity tag. In some embodiments, the affinity tag binds to an immobile substrate. In some embodiments, the immobile substrate is a cellulose substrate.

In some embodiments, the nanobody is conjugated to a drug.

In some embodiments, the nanobody is conjugated to a label.

In some embodiments, the nanobody bind to a target antigen. In some embodiments, the target antigen is a small molecule.

In some embodiments, the small molecule is methotrexate. In some embodiments, the nanobody comprises a nucleic acid sequence that is at least 80%, at least 81%, at least 82%, at least 83%, at least 84%, at least 85%, at least 86%, at least 87%, at least 88%, at least 89, at least 90%, at least 91%, at least 92%, at least 93%, at least 94%, at least 95%, at least 96%, at least 98%, at least 99% identical to SEQ ID NO. 1. In some embodiments, the nanobody comprises SEQ ID NO. 1.

In some embodiments, the small molecule is caffeine. In some embodiments, the nanobody comprises a nucleic acid sequence that is at least 80%, at least 81%, at least 82%, at least 83%, at least 84%, at least 85%, at least 86%, at least 87%, at least 88%, at least 89, at least 90%, at least 91%, at least 92%, at least 93%, at least 94%, at least 95%, at least 96%, at least 98%, at least 99% identical to SEQ ID NO. 2. In some embodiments, the nanobody comprises SEQ ID NO. 2.

In some embodiments the target antigen is a eukaryotic cell surface protein. In some embodiments, the eukaryotic cell surface protein is an immune checkpoint ligand.

In some embodiments, the immune checkpoint ligand is PD-L1. In some embodiments, the nanobody comprises an amino acid sequence that is at least 80%, at least 81%, at least 82%, at least 83%, at least 84%, at least 85%, at least 86%, at least 87%, at least 88%, at least 89, at least 90%, at least 91%, at least 92%, at least 93%, at least 94%, at least 95%, at least 96%, at least 98%, at least 99% identical to SEQ ID NO. 3. In some embodiments, the nanobody comprises SEQ ID NO. 3.

In some embodiments, the immune checkpoint ligand is CTLA-4. In some embodiments, the nanobody comprises an amino acid sequence that is at least 80%, at least 81%, at least 82%, at least 83%, at least 84%, at least 85%, at least 86%, at least 87%, at least 88%, at least 89, at least 90%, at least 91%, at least 92%, at least 93%, at least 94%, at least 95%, at least 96%, at least 98%, at least 99% identical to SEQ ID NO. 4. In some embodiments, the nanobody comprises SEQ ID NO. 4.

In some embodiments, the target antigen is a viral antigen. In some embodiments, the viral antigen is a SARS-CoV-2 antigen. In some embodiments, the SARS-CoV-2 antigen is a spike glycoprotein. In some embodiments, the viral antigen is a hepatitis B antigen. In some embodiments, the hepatitis B antigen is hepatitis B surface antigen (HBsAg). In some embodiments, the hepatitis B antigen is hepatitis B e-antigen (HBeAg). In some embodiments, the hepatitis B antigen is hepatitis B core antigen (HBcAg). In some embodiments, the target antigen is an inflammatory cytokine. In some embodiments, the inflammatory cytokine is TNF-α.

In certain aspects, provided herein is a cell culture supernatant of a Bacillus subtilis strain comprising a nanobody as described herein. In some embodiments, the Bacillus subtilis strain is deficient in at least eight extracellular proteases. In some embodiments, the Bacillus subtilis strain are from a strain comprising at least 90% genomic, 16S and/or CRISPR sequence identity to the nucleotide sequence of the Bacillus subtilis strain WB800N (GenBank Accession No. CP032310.1). In some embodiments, the Bacillus subtilis strain are from a strain comprising at least 99% genomic, 16S and/or CRISPR sequence identity to the nucleotide sequence of the Bacillus subtilis strain WB800N (GenBank Accession No. CP032310.1). In some embodiments, the Bacillus subtilis strain are from Bacillus subtilis strain WB800N (GenBank Accession No. CP032310.1).

In certain aspects, provided herein is a vegetative cell of a Bacillus subtilis strain comprising a nanobody as described herein. In some embodiments, the Bacillus subtilis strain is deficient in at least eight extracellular proteases. In some embodiments, the Bacillus subtilis strain are from a strain comprising at least 90% genomic, 16S and/or CRISPR sequence identity to the nucleotide sequence of the Bacillus subtilis strain WB800N (GenBank Accession No. CP032310.1). In some embodiments, the Bacillus subtilis strain are from a strain comprising at least 99% genomic, 16S and/or CRISPR sequence identity to the nucleotide sequence of the Bacillus subtilis strain WB800N (GenBank Accession No. CP032310.1). In some embodiments, the Bacillus subtilis strain are from Bacillus subtilis strain WB800N (GenBank Accession No. CP032310.1).

In certain aspects, provided herein is a vector comprising a polynucleotide encoding a nanobody as described herein.

In certain aspects, provided herein is a sporulated cell of a Bacillus subtilis strain comprising a vector as described herein. In some embodiments, the Bacillus subtilis strain is deficient in at least eight extracellular proteases. In some embodiments, the Bacillus subtilis strain are from a strain comprising at least 90% genomic, 16S and/or CRISPR sequence identity to the nucleotide sequence of the Bacillus subtilis strain WB800N (GenBank Accession No. CP032310.1). In some embodiments, the Bacillus subtilis strain are from a strain comprising at least 99% genomic, 16S and/or CRISPR sequence identity to the nucleotide sequence of the Bacillus subtilis strain WB800N (GenBank Accession No. CP032310.1). In some embodiments, the Bacillus subtilis strain are from Bacillus subtilis strain WB800N (GenBank Accession No. CP032310.1).

In certain aspects, provided herein is a pharmaceutical composition comprising a nanobody as described herein.

In certain aspects, provided herein is a pharmaceutical composition comprising a cell culture supernatant as described herein.

In certain aspects, provided herein is a pharmaceutical composition comprising a vegetative cell as described herein.

In certain aspects, provided herein is a pharmaceutical composition comprising a sporulated cell as described herein.

In certain aspects, provided herein is use of any one of the pharmaceutical compositions described herein for the preparation of a medicament for treating or prevention of a disease or disorder. In some embodiments, the disease or disorder is a gastrointestinal disease. In some embodiments, the disease or disorder is metabolic disorder. In some embodiments, the disease or disorder is an immunoinflammatory disease. In some embodiments, the disease or disorder is a cancer. In some embodiments, the disease or disorder is an infectious disease or disorder. In some embodiments, the infectious disease is a viral infection. In some embodiments viral infection is COVID-19. In some embodiments, the viral infection is hepatitis B infection. In some embodiments, use of any one of the pharmaceutical compositions described herein further comprises conjointly administering an additional therapeutic to the subject. In some embodiments, the additional therapeutic is an anti-inflammatory agent. In some embodiments, the additional therapeutic is a chemotherapeutic agent. In some embodiments, the additional therapeutic is an immunotherapy agent. In some embodiments, the immunotherapy is an immune checkpoint inhibitor. In some embodiments, the additional therapeutic is an anti-viral agent.

In certain aspects, provided herein is a method of treating a disease or disorder, comprising administering any one of the pharmaceutical compositions as described herein. In some embodiments, the disease or disorder is a gastrointestinal disease. In some embodiments, the disease or disorder is metabolic disorder. In some embodiments, the disease or disorder is an immunoinflammatory disease. In some embodiments, the disease or disorder is a cancer. In some embodiments, the disease or disorder is an infectious disease or disorder. In some embodiments, the infectious disease is a viral infection. In some embodiments viral infection is COVID-19. In some embodiments, the viral infection is hepatitis B infection. In some embodiments, use of any one of the pharmaceutical compositions described herein further comprises conjointly administering an additional therapeutic to the subject. In some embodiments, the additional therapeutic is an anti-inflammatory agent. In some embodiments, the additional therapeutic is a chemotherapeutic agent. In some embodiments, the additional therapeutic is an immunotherapy agent. In some embodiments, the immunotherapy is an immune checkpoint inhibitor. In some embodiments, the additional therapeutic is an anti-viral agent.

In certain aspects, provided herein is a method of inhibiting virus fusion to a human cell, comprising contacting the virus with a nanobody as described herein. In some embodiments, the virus is SARS-CoV-2 virus. In some embodiments, the virus is hepatitis B virus.

In certain aspects, provided herein is a method of producing the nanobody as described herein, comprising the steps of a) expressing the vector as described herein in a Bacillus subtilis strain; and b) harvesting the nanobody from the Bacillus subtilis strain or a cell culture supernatant of the Bacillus subtilis strain. In some embodiments, the nanobody described herein comprises an affinity tag. In some embodiments, the method further comprises isolating the nanobody on an immobile substrate by binding of the affinity tag to the immobile substrate. In some embodiments, the immobile substrate is a cellulose substrate.

In some embodiments, the Bacillus subtilis strain secretes the nanobody. In some embodiments, the Bacillus subtilis strain is deficient in at least eight extracellular proteases. In some embodiments, the Bacillus subtilis strain are from a strain comprising at least 90% genomic, 16S and/or CRISPR sequence identity to the nucleotide sequence of the Bacillus subtilis strain WB800N (GenBank Accession No. CP032310.1). In some embodiments, the Bacillus subtilis strain are from a strain comprising at least 99% genomic, 16S and/or CRISPR sequence identity to the nucleotide sequence of the Bacillus subtilis strain WB800N (GenBank Accession No. CP032310.1). In some embodiments, the Bacillus subtilis strain are from Bacillus subtilis strain WB800N (GenBank Accession No. CP032310.1). In some embodiments, the nanobody is harvested from the cell culture supernatant of the Bacillus subtilis strain. In some embodiments, the Bacillus subtilis strain is a sporulated Bacillus subtilis strain prior to step (a). In some embodiments, the sporulated Bacillus subtilis strain is germinated into a vegetative Bacillus subtilis strain prior to step (a).

In certain aspects, provided herein is a method of detecting the presence of a target antigen in a sample comprising incubating the nanobody as described herein with the sample, wherein the nanobody comprises a detectable label. In some embodiments, the target antigen is a small molecule. In some embodiments, the small molecule is methotrexate. In some embodiments, the small molecule is caffeine. In some embodiments the target antigen is a eukaryotic cell surface protein. In some embodiments, the eukaryotic cell surface protein is an immune checkpoint ligand. In some embodiments, the immune checkpoint ligand is PD-L1. In some embodiments, the immune checkpoint ligand is CTLA-4. In some embodiments, the target antigen is a viral antigen. In some embodiments, the viral antigen is a SARS-CoV-2 antigen. In some embodiments, the SARS-CoV-2 antigen is a spike glycoprotein. In some embodiments, the viral antigen is a hepatitis B antigen. In some embodiments, the hepatitis B antigen is hepatitis B surface antigen (HBsAg). In some embodiments, the hepatitis B antigen is hepatitis B e-antigen (HBeAg). In some embodiments, the hepatitis B antigen is hepatitis B core antigen (HBcAg). In some embodiments, the target antigen is an inflammatory cytokine. In some embodiments, the inflammatory cytokine is TNF-α.

In certain aspects, provided herein is a kit for detecting a target antigen in a sample, comprising a device for collecting the sample and reagents for detecting the target antigen, wherein the reagents comprise the nanobody as described herein and wherein the nanobody comprises a detectable label. In some embodiments, the target antigen is a small molecule. In some embodiments, the small molecule is methotrexate. In some embodiments, the small molecule is caffeine. In some embodiments the target antigen is a eukaryotic cell surface protein. In some embodiments, the eukaryotic cell surface protein is an immune checkpoint ligand. In some embodiments, the immune checkpoint ligand is PD-L1. In some embodiments, the immune checkpoint ligand is CTLA-4. In some embodiments, the target antigen is a viral antigen. In some embodiments, the viral antigen is a SARS-CoV-2 antigen. In some embodiments, the SARS-CoV-2 antigen is a spike glycoprotein. In some embodiments, the viral antigen is a hepatitis B antigen. In some embodiments, the hepatitis B antigen is hepatitis B surface antigen (HBsAg). In some embodiments, the hepatitis B antigen is hepatitis B e-antigen (HBeAg). In some embodiments, the hepatitis B antigen is hepatitis B core antigen (HBcAg). In some embodiments, the target antigen is an inflammatory cytokine. In some embodiments, the inflammatory cytokine is TNF-α.

### BRIEF DESCRIPTION OF THE FIGURES

FIG. 1 shows a schematic to illustrate the structure and properties of B. subtilis spores as stable living systems to manufacture camelid-derived nanobodies on demand. These spores are extremely resistant to space vacuum, cosmic radiation, temperature fluctuations, UV (if shielded) or acidic pH.

FIG. 2 shows a schematic pipeline for nanobody production, including dry spore storage, inoculation of spores into media to allow for spore germination and outgrowth, bacterial growth and protein secretion and purification. Abbreviations: amyQ, protein secretion signal peptide; groE, a strong promoter from the B. subtilis groE operon which has been converted into an IPTG-inducible promoter by addition of the lac operator from E. coli.

FIG. 3 shows extracellular secretion of nanobodies in B. subtilis. Panel A shows a schematic to use B. subtilis to produce camelid-derived nanobodies. Panel B shows Sodium Dodecyl Sulfate Polyacrylamide Gel Electrophoresis (SDS-PAGE) analysis of B. subtilis strains engineered to secrete four different nanobodies upon growth starting from dormant spores. The nanobodies are (1) anti-caffeine; (2) anti-methotrexate (MTX); (3) anti-Cytotoxic T-Lymphocyte Associated Protein 4 (CTLA-4, mouse) and (4) anti-Programmed Death-Ligand 1 (PD-L1, mouse). The image shown is an SDS-PAGE gel of total proteins secreted by B. subtilis and precipitated by trichloroacetic acid (TCA) after induction with 1 mM IPTG overnight. The rectangles indicate the target proteins which are absent in the noninduced culture. The theoretical molecular weight of each fusion protein is indicated at the bottom of the SDS-PAGE gel. Panel C shows an exemplary Western blot to confirm the presence of target proteins. Recombinant proteins were fused with a FLAG epitope for detection by an anti-FLAG antibody. Panel D shows SDS-PAGE analysis of the kinetics of kinetics of B. subtilis secretion of an anti-caffeine nanobody fused with a CBD after IPTG induction.

FIG. 4 shows cellulose binding and nanobody-specific target recognition of CBD fusion proteins. Panel A shows a schematic to detect recombinant fusion proteins after dropping bacterial supernatant on cellulose-based filter paper. Panel B shows binding of fusion proteins to cellulose filter paper. Panel C shows patterning of a supernatant from a 4 hr induction on a filter paper. Panel D shows immobilization and stability of CBD-anti-caffeine nanobody fusion protein after three months. Panel E shows percentage of remaining caffeine in five continuous flow-through fractions (100 μl per fraction) relative to the starting concentration (150 μg/ml in PBS).

FIG. 5 shows detection of protein antigens using nanobodies secreted from B. subtilis. Panel A shows a scheme to use two different cell surface antigens (PD-L1 and CTLA-4) to validate Nbs secreted from B. subtilis. Panel B shows recombinant nanobody anti-PD-L1 purified from the supernatant of B. subtilis cells expressing this protein can readily detect PD-L1 on the surface of an immortalized murine dendritic cell line, DC 2.4. Panel C shows direct detection of the cell surface antigen PD-L1 by bacterial supernatant. Panel D shows exemplary immunofluorescence micrographs that show at recombinant Nb anti-PD-L1, but not the anti-caffeine Nb, can detect PD-L1 expression on the surface of DC 2.4 dendritic cells as purple fluorescence. Panel E shows detection of CTLA-4 positive cells in unstimulated primary cells harvested from lymph nodes in C57BL/6 mice.

FIG. 6 shows development of a highly resistant B. subtilis spore-based biologic production platform. Panel A shows exemplary phase contrast micrograph of spores encoding CTLA-4 after the Histodenz-based density gradient purification. Arrowheads indicate individual spores. Panel B shows B. subtilis spores encoding anti-CTLA-4 nanobodies are resistant to desiccation air drying on filter paper for 24 and 48 hr at 37° C., while vegetative cells are not resistant. Panel C shows spores with desiccation treatment for 24 and 48 hr maintain the ability to produce the recombinant nanobodies anti-CTLA-4 and anti-PD-L1 without noticeable losses in yield in comparison to the untreated group. Panel D shows spores expressing anti-CTLA-4 nanobodie are tolerant to wet heat (80° C.) for up to 3 hr, while vegetative cells are killed within 1 minute. Panel E shows spores with heat treatment for 3 min and 3 hr maintain the ability to produce the recombinant nanobodies anti-CTLA-4 and anti-PD-L1 without noticeable losses in yield in comparison to the untreated group. Panel F shows Spores are resistant to acidic pH, while vegetative cells are not. LB plates show growth of B. subtilis spores or vegetative cells after acid challenges at pH 1.1 and 2.9 for the indicated times. Panel G shows B. subtilis spores are more resistant to ultraviolet (UV) irradiation at 254 nm than vegetative cells. In contrast, spores and vegetative cells survived exposure to UV light at 365 nm for up to 40 min, without noticeable differences in viability.

FIG. 7 shows schematic of COVID-19 diagnosis on a swab using cellulose-based COVID-19 capturing nanobodies. The nanobodies are immobilized on cotton swabs to efficiently capture viral particles from different surfaces. Moreover, COVID-19 virus (SARS-CoV-2) captured by the functional swabs can be readily integrated with immune assays and smart phones for detection.

### DETAILED DESCRIPTION

### Definitions

“Adjuvant” or “Adjuvant therapy” broadly refers to an agent that affects an immunological or physiological response in a patient or subject (e.g., human). For example, an adjuvant might increase the presence of an antigen over time or to an area of interest like a tumor, help absorb an antigen presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent.

“Administration” broadly refers to a route of administration of a composition (e.g., a pharmaceutical composition) to a subject. Examples of routes of administration include oral administration, rectal administration, topical administration, inhalation (nasal) or injection. Administration by injection includes intravenous (IV), intramuscular (IM), intratumoral (IT) and subcutaneous (SC) administration. A pharmaceutical composition described herein can be administered in any form by any effective route, including but not limited to intratumoral, oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), implanted, intravesical, intrapulmonary, intraduodenal, intragastrical, and intrabronchial. In preferred embodiments, a pharmaceutical composition described herein is administered orally, rectally, intratumorally, topically, intravesically, by injection into or adjacent to a draining lymph node, intravenously, by inhalation or aerosol, or subcutaneously. In another preferred embodiment, a pharmaceutical composition described herein is administered orally, intratumorally, or intravenously.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as VH) and a heavy chain constant region. Each light chain includes a light chain variable region (abbreviated herein as VL) and a light chain constant region. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The terms “antigen binding fragment” and “antigen-binding portion” of an antibody, as used herein, refer to one or more fragments of an antibody that retain the ability to bind to an antigen. Examples of binding fragments encompassed within the term “antigen-binding fragment” of an antibody include Fab, Fab′, F(ab′)2, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

“Cancer” broadly refers to an uncontrolled, abnormal growth of a host's own cells leading to invasion of surrounding tissue and potentially tissue distal to the initial site of abnormal cell growth in the host. Major classes include carcinomas which are cancers of the epithelial tissue (e.g., skin, squamous cells); sarcomas which are cancers of the connective tissue (e.g., bone, cartilage, fat, muscle, blood vessels, etc.); leukemias which are cancers of blood forming tissue (e.g., bone marrow tissue); lymphomas and myelomas which are cancers of immune cells; and central nervous system cancers which include cancers from brain and spinal tissue. “Cancer(s) and” “neoplasm(s)”” are used herein interchangeably. As used herein, “cancer” refers to all types of cancer or neoplasm or malignant tumors including leukemias, carcinomas and sarcomas, whether new or recurring. Specific examples of cancers are: carcinomas, sarcomas, myelomas, leukemias, lymphomas and mixed type tumors. Non-limiting examples of cancers are new or recurring cancers of the brain, melanoma, bladder, breast, cervix, colon, head and neck, kidney, lung, non-small cell lung, mesothelioma, ovary, prostate, sarcoma, stomach, uterus and medulloblastoma. In some embodiments, the cancer comprises a solid tumor. In some embodiments, the cancer comprises a metastasis.

“Clade” refers to the OTUs or members of a phylogenetic tree that are downstream of a statistically valid node in a phylogenetic tree. The clade comprises a set of terminal leaves in the phylogenetic tree that is a distinct monophyletic evolutionary unit and that share some extent of sequence similarity.

As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, an individual who receives such treatment can benefit from a combined effect of different therapeutic compounds.

The term “decrease” or “deplete” means a change, such that the difference is, depending on circumstances, at least 10%, 20%, 30%, 40%, 50%, 60%, 70%, 80%, 90%, 1/100, 1/1000, 1/10,000, 1/100,000, 1/1,000,000 or undetectable after treatment when compared to a pre-treatment state.

The term “ecological consortium” is a group of bacteria which trades metabolites and positively co-regulates one another, in contrast to two bacteria which induce host synergy through activating complementary host pathways for improved efficacy.

As used herein, “engineered bacteria” are any bacteria that have been genetically altered from their natural state by human activities, and the progeny of any such bacteria. Engineered bacteria include, for example, the products of targeted genetic modification, the products of random mutagenesis screens and the products of directed evolution.

The term “epitope” means a protein determinant capable of specific binding to an antibody or nanobody or T cell receptor. Epitopes usually consist of chemically active surface groupings of molecules such as amino acids or sugar side chains. Certain epitopes can be defined by a particular sequence of amino acids to which an antibody or nanobody is capable of binding.

The term “gene” is used broadly to refer to any nucleic acid associated with a biological function. The term “gene” applies to a specific genomic sequence, as well as to a cDNA or an mRNA encoded by that genomic sequence.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Mrtin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

As used herein, the term “immune disorder” refers to any disease, disorder or disease symptom caused by an activity of the immune system, including autoimmune diseases, inflammatory diseases and allergies. Immune disorders include, but are not limited to, autoimmune diseases (e.g., psoriasis, atopic dermatitis, lupus, scleroderma, hemolytic anemia, vasculitis, type one diabetes, Grave's disease, rheumatoid arthritis, multiple sclerosis, Goodpasture's syndrome, pernicious anemia and/or myopathy), inflammatory diseases (e.g., acne vulgaris, asthma, celiac disease, chronic prostatitis, glomerulonephritis, inflammatory bowel disease, pelvic inflammatory disease, reperfusion injury, rheumatoid arthritis, sarcoidosis, transplant rejection, vasculitis and/or interstitial cystitis), and/or an allergies (e.g., food allergies, drug allergies and/or environmental allergies).

“Immunotherapy” is treatment that uses a subject's immune system to treat disease (e.g., immune disease, inflammatory disease, metabolic disease, cancer) and includes, for example, checkpoint inhibitors, cancer vaccines, cytokines, cell therapy, CAR-T cells, and dendritic cell therapy.

The term “increase” means a change, such that the difference is, depending on circumstances, at least 10%, 20%, 30%, 40%, 50%, 60%, 70%, 80%, 90%, 2-fold, 4-fold, 10-fold, 100-fold, 10\hat{ }3 fold, 10\hat{ }4 fold, 10\hat{ }5 fold, 10\hat{ }6 fold, and/or 10\hat{ }7 fold greater after treatment when compared to a pre-treatment state. Properties that may be increased include immune cells, bacterial cells, stromal cells, myeloid derived suppressor cells, fibroblasts, metabolites, and cytokines.

“Innate immune agonists” or “immuno-adjuvants” are small molecules, proteins, or other agents that specifically target innate immune receptors including Toll-Like Receptors (TLR), NOD receptors, RLRs, C-type lectin receptors, STING-cGAS Pathway components, inflammasome complexes. For example, LPS is a TLR-4 agonist that is bacterially derived or synthesized and aluminum can be used as an immune stimulating adjuvant. immuno-adjuvants are a specific class of broader adjuvant or adjuvant therapy. Examples of STING agonists include, but are not limited to, 2′3′-cGAMP, 3′3′-cGAMP, c-di-AMP, c-di-GMP, 2′2′-cGAMP, and 2′3′-cGAM(PS)2 (Rp/Sp) (Rp, Sp-isomers of the bis-phosphorothioate analog of 2′3′-cGAMP). Examples of TLR agonists include, but are not limited to, TLR1, TLR2, TLR3, TLR4, TLR5, TLR6, TLR7, TLR8, TLR9, TLR1O and TLRI 1. Examples of NOD agonists include, but are not limited to, N-acetylmuramyl-L-alanyl-D-isoglutamine (muramyldipeptide (MDP)), gamma-D-glutamyl-meso-diaminopimelic acid (iE-DAP), and desmuramylpeptides (DMP).

The term “isolated” or “enriched” encompasses a microbe, a nanobody, or other entity or substance that has been (1) separated from at least some of the components with which it was associated when initially produced (whether in nature or in an experimental setting), and/or (2) produced, prepared, purified, and/or manufactured by the hand of man. Isolated microbes or nanobodies may be separated from at least about 10%, about 20%, about 30%, about 40%, about 50%, about 60%, about 70%, about 80%, about 90%, or more of the other components with which they were initially associated. In some embodiments, isolated microbes or nanobodies are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. The terms “purify,” “purifying” and “purified” refer to a microbe or nanobody or other material that has been separated from at least some of the components with which it was associated either when initially produced or generated (e.g., whether in nature or in an experimental setting), or during any time after its initial production. A microbe or a microbial population or nanobody may be considered purified if it is isolated at or after production, such as from a material or environment containing the microbe or microbial population or nanobody, and a purified microbe or microbial or nanobody population may contain other materials up to about 10%, about 20%, about 30%, about 40%, about 50%, about 60%, about 70%, about 80%, about 90%, or above about 90% and still be considered “isolated.” In some embodiments, purified microbes or nanobodies or microbial population are more than about 80%, about 85%, about 90%, about 91%, about 92%, about 93%, about 94%, about 95%, about 96%, about 97%, about 98%, about 99%, or more than about 99% pure. In the instance of microbial compositions provided herein, the one or more microbial types present in the composition can be independently purified from one or more other microbes produced and/or present in the material or environment containing the microbial type. Microbial compositions and the microbial components such as nanobodies thereof are generally purified from residual habitat products.

“Microbe” refers to any natural or engineered organism characterized as a archaeaon, parasite, bacterium, fungus, microscopic alga, protozoan, and the stages of development or life cycle stages (e.g., vegetative, spore (including sporulation, dormancy, and germination), latent, biofilm) associated with the organism. Examples of gut microbes include: Actinomyces graevenitzii, Actinomyces odontolyticus, Akkermansia muciniphila, Bacillus subtilis, Bacteroides caccae, Bacteroides fragilis, Bacteroides putredinis, Bacteroides thetaiotaomicron, Bacteroides vultagus, Bifdobacterium adolescentis, Bifdobacterium bifidum, Bilophila wadsworthia, Blautia, Butyrivibrio, Campylobacter gracilis, Clostridia cluster III, Clostridia cluster IV, Clostridia cluster IX (Acidaminococcaceae group), Clostridia cluster A, Clostridia cluster XIII (Peptostreptococcus group), Clostridia cluster XIV, Clostridia cluster XV, Collinsella aerofaciens, Coprococcus, Corynebacterium sunsvallense, Desulfomonas pigra, Dorea formicigenerans, Dorea longicatena, Escherichia coli, Eubacterium hadrum, Eubacterium rectale, Faecalibacteria prausnitzii, Gemella, Lactococcus, Lanchnospira, Mollicutes cluster XVI, Mollicutes cluster XVIII, Prevotella, Rothia mucilaginosa, Ruminococcus callidus, Ruminococcus gnavus, Ruminococcus torques, and Streptococcus.

“Microbiome” broadly refers to the microbes residing on or in body site of a subject or patient. Microbes in a microbiome may include bacteria, viruses, eukaryotic microorganisms, and/or viruses. Individual microbes in a microbiome may be metabolically active, dormant, latent, or exist as spores, may exist planktonically or in biofilms, or may be present in the microbiome in sustainable or transient manner. The microbiome may be a commensal or healthy-state microbiome or a disease-state microbiome. The microbiome may be native to the subject or patient, or components of the microbiome may be modulated, introduced, or depleted due to changes in health state (e.g., precancerous or cancerous state) or treatment conditions (e.g., antibiotic treatment, exposure to different microbes). In some aspects, the microbiome occurs at a mucosal surface. In some aspects, the microbiome is a gut microbiome. In some aspects, the microbiome is a tumor microbiome.

A “microbiome profile” or a “microbiome signature” of a tissue or sample refers to an at least partial characterization of the bacterial makeup of a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more bacterial strains are present or absent in a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more cancer-associated bacterial strains are present in a sample. In some embodiments, the microbiome profile indicates the relative or absolute amount of each bacterial strain detected in the sample. In some embodiments, the microbiome profile is a cancer-associated microbiome profile. A cancer-associated microbiome profile is a microbiome profile that occurs with greater frequency in a subject who has cancer than in the general population. In some embodiments, the cancer-associated microbiome profile comprises a greater number of or amount of cancer-associated bacteria than is normally present in a microbiome of an otherwise equivalent tissue or sample taken from an individual who does not have cancer.

“Modified” in reference to a bacteria broadly refers to a bacteria that has undergone a change from its wild-type form. Bacterial modification can result from engineering bacteria. Examples of bacterial modifications include genetic modification, gene expression modification, phenotype modification, formulation modification, chemical modification, and dose or concentration. Examples of improved properties are described throughout this specification and include, e.g., attenuation, auxotrophy, homing, or antigenicity. Phenotype modification might include, by way of example, bacteria growth in media that modify the phenotype of a bacterium such that it increases or decreases virulence.

An “oncobiome” as used herein comprises tumorigenic and/or cancer-associated microbiota, wherein the microbiota comprises one or more of a virus, a bacterium, a fungus, a protist, a parasite, or another microbe.

“Oncotrophic” or “oncophilic” microbes and bacteria are microbes that are highly associated or present in a cancer microenvironment. They may be preferentially selected for within the environment, preferentially grow in a cancer microenvironment or hone to a said environment.

“Operational taxonomic units” and “OTU(s)” refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. For 16S, OTUs that share ≥97% average nucleotide identity across the entire 16S or some variable region of the 16S are considered the same OTU. See e.g., Claesson M J, Wang Q, O'Sullivan O, Greene-Diniz R, Cole J R, Ross R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940. For complete genomes, MLSTs, specific genes, other than 16S, or sets of genes OTUs that share ≥95% average nucleotide identity are considered the same OTU. See e.g., Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940. OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Operational Taxonomic Units (OTUs) with taxonomic assignments made to, e.g., genus, species, and phylogenetic clade are provided herein.

As used herein, a gene is “overexpressed” in a bacteria if it is expressed at a higher level in an engineered bacteria under at least some conditions than it is expressed by a wild-type bacteria of the same species under the same conditions. Similarly, a gene is “underexpressed” in a bacteria if it is expressed at a lower level in an engineered bacteria under at least some conditions than it is expressed by a wild-type bacteria of the same species under the same conditions.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), micro RNA (miRNA), silencing RNA (siRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, a substance is “pure” if it is substantially free of other components. The terms “purify,” “purifying” and “purified” refer to a nanobody preparation or other material that has been separated from at least some of the components with which it was associated either when initially produced or generated (e.g., whether in nature or in an experimental setting), or during any time after its initial production. A nanobody preparation or compositions may be considered purified if it is isolated at or after production, such as from one or more other bacterial components, and a purified microbe or microbial population may contain other materials up to about 10%, about 20%, about 30%, about 40%, about 50%, about 60%, about 70%, about 80%, about 90%, or above about 90% and still be considered “purified.” In some embodiments, purified nanobodies are more than about 80%, about 85%, about 90%, about 91%, about 92%, about 93%, about 94%, about 95%, about 96%, about 97%, about 98%, about 99%, or more than about 99% pure.

As used herein, the term “purified nanobody composition” or “nanobody composition” refers to a preparation that includes nanobodies that have been separated from at least one associated substance found in a source material (e.g., separated from at least one other bacterial component) or any material associated with the nanobodies in any process used to produce the preparation. It also refers to a composition that has been significantly enriched or concentrated. In some embodiments, the nanobodies are concentrated by 2 fold, 3-fold, 4-fold, 5-fold, 10-fold, 100-fold, 1000-fold, 10,000-fold or more than 10,000 fold.

As used herein, “specific binding” refers to the ability of an antibody or nanobody to bind to a predetermined antigen or the ability of a polypeptide to bind to its predetermined binding partner. Typically, an antibody or nanobody or polypeptide specifically binds to its predetermined antigen or binding partner with an affinity corresponding to a K_{D} of about 10⁴ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein). Alternatively, specific binding applies more broadly to a two component system where one component is a protein, lipid, or carbohydrate or combination thereof and engages with the second component which is a protein, lipid, carbohydrate or combination thereof in a specific way.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

The terms “subject” or “patient” refers to any mammal. A subject or a patient described as “in need thereof” refers to one in need of a treatment (or prevention) for a disease. Mammals (i.e., mammalian animals) include humans, laboratory animals (e.g., primates, rats, mice), livestock (e.g., cows, sheep, goats, pigs), and household pets (e.g., dogs, cats, rodents). The subject may be a human. The subject may be a non-human mammal including but not limited to of a dog, a cat, a cow, a horse, a pig, a donkey, a goat, a camel, a mouse, a rat, a guinea pig, a sheep, a llama, a monkey, a gorilla or a chimpanzee. The subject may be healthy, or may be suffering from a cancer at any developmental stage, wherein any of the stages are either caused by or opportunistically supported of a cancer associated or causative pathogen, or may be at risk of developing a cancer, or transmitting to others a cancer associated or cancer causative pathogen. In some embodiments, a subject has lung cancer, bladder cancer, prostate cancer, plasmacytoma, colorectal cancer, rectal cancer, Merkel Cell carcinoma, salivary gland carcinoma, ovarian cancer, and/or melanoma. The subject may have a tumor. The subject may have a tumor that shows enhanced macropinocytosis with the underlying genomics of this process including Ras activation. In other embodiments, the subject has another cancer. In some embodiments, the subject has undergone a cancer therapy.

As used herein, the term “treating” a disease in a subject or “treating” a subject having or suspected of having a disease refers to administering to the subject to a pharmaceutical treatment, e.g., the administration of one or more agents, such that at least one symptom of the disease is decreased or prevented from worsening. Thus, in one embodiment, “treating” refers inter alia to delaying progression, expediting remission, inducing remission, augmenting remission, speeding recovery, increasing efficacy of or decreasing resistance to alternative therapeutics, or a combination thereof. As used herein, the term “preventing” a disease in a subject refers to administering to the subject to a pharmaceutical treatment, e.g., the administration of one or more agents, such that onset of at least one symptom of the disease is delayed or prevented.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The terms “single domain antibody” or “nanobody” refer to an antibody comprising one variable domain of a antibody. In some embodiments the variable domain is obtained from a heavy-chain antibody (VHH) of camelids or fish. Antibody proteins obtained from members of the camelid family include new world members such as llama species (Lama paccos, Lama glama and Lama vicugna) that have been characterized with respect to size, structural complexity and antigenicity for human subjects. Certain IgG antibodies from this family of mammals as found in nature lack light chains, and are thus structurally distinct from the typical four chain quaternary structure having two heavy and two light chains, for antibodies from other animals. See, PCT/EP93/02214 (published as WO 94/04678).

The term ‘VHH’ refers to single heavy chain variable domain antibodies devoid of light chains. Generally, a VHH antibody is an antibody of the type that can be found in Camelidae or cartilaginous fish which are naturally devoid of light chains or to a synthetic and non-immunized VHH which can be constructed accordingly. Each heavy chain comprises a variable region encoded by V-, D- and J exons. The VHH may be a natural VHH antibody, preferably a Camelid antibody, or a recombinant protein comprising a heavy chain variable domain.

Bacillus subtilis Bacteria

The Bacillus species are rod-shaped, spore-forming, aerobic, gram-positive bacteria that are ubiquitous in nature. It is mostly found in soil and vegetation with an optimal growth temperature from 25-35 degrees Celsius. There is some evidence that Bacillus subtilis might be a part of the normal gut flora of humans. Some human intestinal biopsy samples have shown that Bacillus subtilis populates the gut in humans as intestinal flora. See, e.g., Junjie Qin, et al., A human gut microbial gene catalogue established by metagenomics sequencing, Nature 464, 59-65 (2010), incorporated by reference herein in its entirety.

B. subtilis has the ability to produce and secrete proteins via its secretion system (Sec pathway). Spores of B. subtilis can tolerate harsh environmental conditions, such as UV exposure and high temperatures. Sporulation begins when a sporangium divides asymmetrically to produce two compartments: the mother cell and the forespore, which are separated by a septum. Next, the mother cell engulfs the forespore, and following membrane fission at the opposite pole of the sporangium, a double-membrane bound forespore is formed. Coat assembly begins just after the initiation of engulfment and continues throughout sporulation. The peptidoglycan cortex between the inner and outer forespore membranes is assembled during late sporulation. In the final step, the mother cell lyses to release a mature spore into the environment. Spores are capable of quickly germinating and resuming vegetative growth in response to nutrients.

In certain aspects, provided herein are nanobodies contained within Bacillus subtilis bacteria. In some embodiments, the Bacillus subtilis bacteria from which the nanobodies are contained within are modified to reduce toxicity or other adverse effects, to enhance delivery) (e.g., oral delivery) of the nanobodies (e.g., by improving acid resistance, muco-adherence and/or penetration and/or resistance to bile acids, digestive enzymes, resistance to anti-microbial peptides and/or antibody neutralization), to target desired cell types (e.g., M-cells, goblet cells, enterocytes, dendritic cells, macrophages), to enhance their immunomodulatory and/or therapeutic effect of the nanobodies (e.g., either alone or in combination with another therapeutic agent), and/or to enhance immune activation or suppression by the nanobodies (e.g., through modified production of polysaccharides, pili, fimbriae, adhesins). In some embodiments, the engineered Bacillus subtilis described herein are modified to improve nanobodies manufacturing (e.g., higher oxygen tolerance, stability, improved freeze-thaw tolerance, shorter generation times). For example, in some embodiments, the engineered Bacillus subtilis described include Bacillus subtilis harboring one or more genetic changes, such change being an insertion, deletion, translocation, or substitution, or any combination thereof, of one or more nucleotides contained on the bacterial chromosome or endogenous plasmid vector and/or one or more foreign plasmid vectors, wherein the genetic change may results in the overexpression and/or underexpression of one or more genes. The engineered Bacillus subtilis may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, or any combination thereof.

An exemplary Bacillus subtilis strain that can be used as a source of nanobodies described herein is provided in Table 1. In some embodiments, Bacillus subtilis bacteria from which nanobodies are obtained are lyophilized.

In some embodiments, Bacillus subtilis bacteria from which nanobodies are obtained are gamma irradiated (e.g., at 17.5 or 25 kGy).

In some embodiments, Bacillus subtilis bacteria from which nanobodies are obtained are UV irradiated.

In some embodiments, Bacillus subtilis bacteria from which nanobodies are obtained are heat inactivated (e.g., at 50° C. for two hours or at 90° C. for two hours).

The phase of growth can affect the amount or properties of bacteria and/or nanobodies produced by bacteria. For example, in the methods of nanobodies preparation provided herein, nanobodies can be isolated, e.g., from a culture, at the start of the log phase of growth, midway through the log phase, and/or once stationary phase growth has been reached.

In some embodiments, the Bacillus subtilis bacteria are cultured to high optical density and then centrifuged to pellet bacteria (e.g., at 10,000×g for 30 min at 4° C., at 15,500×g for 15 min at 4° C.). In some embodiments, the culture supernatants are then passed through filters to exclude intact bacterial cells (e.g., a 0.22 μm filter). In some embodiments, filtered supernatants are centrifuged to pellet nanobodies (e.g., at 100,000-150,000×g for 1-3 hours at 4° C., at 200,000×g for 1-3 hours at 4° C.).

In the methods of nanobody preparation provided herein, the method can optionally include exposing a culture of bacteria to a nanobody inducer prior to isolating nanobodies from the Bacillus subtilis bacterial culture. The culture of Bacillus subtilis bacteria can be exposed to a nanobody inducer at the start of the log phase of growth, midway through the log phase, and/or once stationary phase growth has been reached.

TABLE 1
Exemplary Bacillus subtilis Bacterial Strain

| OTU | Public Repository Accession | Strain |
|---|---|---|
| Bacillus subtilis | EU627588 | WB800N (GenBank |
| | | Accession No. |
| | | CP032310.1) |

In some embodiments, the Bacillus subtilis strain is deficient in at least eight extracellular proteases.

In some embodiments, the nanobodies are contained within a Bacillus subtilis strain comprising at least 90%, at least 91%, at least 92%, at least 93%, at least 94%, at least 95%, at least 96%, at least 97%, at least 98%, or at least 99% genomic, 16S and/or CRISPR sequence identity to the nucleotide sequence of the Bacillus subtilis strain WB800N (GenBank Accession No. CP032310.1). In some embodiments, the nanobodies are contained within a Bacillus subtilis strain WB800N (GenBank Accession No. CP032310.1).

In some embodiments the Bacillus subtilis strain secretes the nanobody.

In certain aspects, provided herein is a cell culture supernatant of a Bacillus subtilis strain comprising a nanobody as described herein. In some embodiments, the Bacillus subtilis strain is deficient in at least eight extracellular proteases. In some embodiments, the Bacillus subtilis strain are from a strain comprising at least 90% genomic, 16S and/or CRISPR sequence identity to the nucleotide sequence of the Bacillus subtilis strain WB800N (GenBank Accession No. CP032310.1). In some embodiments, the Bacillus subtilis strain are from a strain comprising at least 99% genomic, 16S and/or CRISPR sequence identity to the nucleotide sequence of the Bacillus subtilis strain WB800N (GenBank Accession No. CP032310.1). In some embodiments, the Bacillus subtilis strain are from Bacillus subtilis strain WB800N (GenBank Accession No. CP032310.1).

In certain aspects, provided herein is a vegetative cell of a Bacillus subtilis strain comprising a nanobody as described herein. In some embodiments, the Bacillus subtilis strain is deficient in at least eight extracellular proteases. In some embodiments, the Bacillus subtilis strain are from a strain comprising at least 90% genomic, 16S and/or CRISPR sequence identity to the nucleotide sequence of the Bacillus subtilis strain WB800N (GenBank Accession No. CP032310.1). In some embodiments, the Bacillus subtilis strain are from a strain comprising at least 99% genomic, 16S and/or CRISPR sequence identity to the nucleotide sequence of the Bacillus subtilis strain WB800N (GenBank Accession No. CP032310.1). In some embodiments, the Bacillus subtilis strain are from Bacillus subtilis strain WB800N (GenBank Accession No. CP032310.1).

In certain aspects, provided herein is a vector comprising a polynucleotide encoding a nanobody as described herein.

Sporulation of Bacillus subtilis may be caused by environmental stress. Methods of sporulation are known to those skilled in the art. Methods of sporulation include, but are not limited to, heat inactivation (e.g., subject Bacillus subtilis bacteria to high temperature), UV irradiation, desiccation, chemical damage and enzymatic destruction.

In certain aspects, provided herein is a sporulated cell of a Bacillus subtilis strain comprising a vector as described herein. In some embodiments, the Bacillus subtilis strain is deficient in at least eight extracellular proteases. In some embodiments, the Bacillus subtilis strain are from a strain comprising at least 90% genomic, 16S and/or CRISPR sequence identity to the nucleotide sequence of the Bacillus subtilis strain WB800N (GenBank Accession No. CP032310.1). In some embodiments, the Bacillus subtilis strain are from a strain comprising at least 99% genomic, 16S and/or CRISPR sequence identity to the nucleotide sequence of the Bacillus subtilis strain WB800N (GenBank Accession No. CP032310.1). In some embodiments, the Bacillus subtilis strain are from Bacillus subtilis strain WB800N (GenBank Accession No. CP032310.1).

In some embodiments, the sporulated cell of a Bacillus subtilis strain is prepared and/or used for long-term storage.

### Nanobodies

Nanobodies are single domain antibodies (sdAb) typically consisting of a single monomeric variable antibody domain. Like whole antibodies (intact immunoglobulins), nanobodies are able to bind selectively to a specific antigen. With a molecular weight typically ranging from about 12 kDa to about 15 kDa, the single-domain nanobodies are much smaller than intact immunoglobulins which are typically composed of two heavy protein chains and two light chains. Nanobodies are also typically smaller than Fab fragments (about 50 kDa, one light chain and half a heavy chain) and single-chain variable fragments (about 25 kDa, two variable domains, one from a light and one from a heavy chain).

Methods of producing nanobodies are described, inter alia, by Harmsen and Haard (2007) Appl. Microbiol. Biotechnol. 77 (1): 13-22).

Initially, nanobodies were engineered from heavy-chain antibodies found in camelids. These are called VHH fragments. Cartilaginous fishes also have heavy-chain antibodies (immunoglobulin new antigen receptor (IgNAR)′), from which single-domain antibodies called VNAR fragments can be obtained. An alternative approach is to split the dimeric variable domains from a common human or other mammal (e.g., mice, rabbits, etc.) into monomers. Although most research into single-domain antibodies is based on heavy chain variable domains, nanobodies derived from light chains have also been shown to bind specifically to target epitopes (see, e.g., Moller et al. (2010) J. Biol. Chem. 285(49): 38348-38361). Single-domain camelids antibodies have been shown to be just as specific as a regular antibody and in some cases they are more robust. As well, they are easily isolated using the same phage panning procedure used for traditional antibodies, allowing them to cultured in vitro in large concentrations. The smaller size and single domain make these antibodies easier to transform into bacterial cells for bulk production, making them particularly useful for research purposes.

Typically the single-domain antibody is a peptide chain about 110 amino acids long, comprising one variable domain (VH) of a heavy-chain antibody, or of a common IgG. These peptides have similar affinity to antigens as whole antibodies, but are more heat-resistant and stable towards detergents and high concentrations of urea. Those derived from camelid and fish antibodies are less lipophilic and more soluble in water, which, without being bound to a particular theory, is believed to be due to their complementarity determining region 3 (CDR3), which forms an extended loop covering the lipophilic site that normally binds to a light chain (see, e.g., Dolk et al. (2005) Appl. Environ. Microbiol. 71(1): 442-450; Stanfield et al. (2004) Science, 305(5691): 1770-1773).

The comparatively low molecular mass of nanobodies often leads to better permeability in tissues, and to a short plasma half-life since they are eliminated renally. Unlike whole antibodies, they do not show complement system triggered cytotoxicity because they lack an Fc region. However, in certain embodiments, it is contemplated that an immunoglobulin Fc region (or variant Fc region) can be fused to the nanobody to provide additional functionality. Camelid and fish derived sdAbs are able to bind to hidden antigens that are may not be accessible to whole antibodies, for example to the active sites of enzymes. It is believed that this property has been shown to result from their extended CDR3 loop, which is able to penetrate such sites (see, e.g., Stanfield et al. (2004) Science 305(5691): 1770-1773; Desmyter et al. (1996) Nat. Struct. Biol. 3(9): 803-811).

In certain aspects, provided herein is a nanobody comprising a variable domain of an antibody, wherein the nanobody is contained within a Bacillus subtilis bacteria strain. In some embodiments the variable domain is a heavy chain variable domain. In some embodiments, the antibody is a mammalian antibody. In some embodiments, the mammalian antibody is a camelid antibody. In some embodiments, the antibody is a fish antibody.

In some embodiments, the nanobody comprises an affinity tag. In some embodiments, the affinity tag binds to an immobile substrate. In some embodiments, the immobile substrate is a cellulose substrate.

Antibody drug conjugates (ADC) represent a new class of therapeutics comprising an antibody conjugated to a cytotoxic drug via a chemical linker. A number of ADCs have been tested in clinical trials today, including trastuzumab (Herceptin (anti-HER2 antibody) linked to DM1; Genentech/Roche) and glembatumumab vedotin (CDX-011; an anti-GPNMB antibody linked to MMAE; Celldex Therapeutics).

In some embodiments, the nanobody is conjugated to a drug. In some embodiments, the nanobody is an antibody drug conjugate.

In some embodiments, the nanobody is conjugated to a label. Exemplary labels include, but are not limited to, radiolabels such as the isotopes 2H, 3H, 11C, 13C, 14C, 32P, 33S, 34S, 35S, 36S, 36Cl, 51Cr, 57Co, 58Co, 59Fe, 90Y, 121I, 124I, 125I, 131I, 211At, 198Au, 67Cu, 225Ac, 213Bi, 99Tc and 186Re, which may be attached to nanobodies using conventional chemistry known in the art of antibody imaging. Labels also include fluorescent labels and enzyme labels such as horseradish peroxidase. Exemplary fluorescent labels are listed in Table 2. Labels further include chemical moieties such as biotin which may be detected via binding to a specific cognate detectable moiety, e.g. labeled avidin.

In some embodiments, the nanobodies are lyophilized.

TABLE 2
Exemplary Fluorescent Labels

| | Absorbance | Emission | Visible |
|---|---|---|---|
| Dye | Wavelength | Wavelength | color |
| Hydroxycoumarin | 325 | 386 | blue |
| methoxycoumarin | 360 | 410 | blue |
| Alexa Fluor 345 | 345 | 442 | blue |
| aminocoumarin | 350 | 445 | blue |
| Cy2 | 490 | 510 | green (dark) |
| FAM | 495 | 516 | green (dark) |
| Alexa Fluor 488 | 494 | 517 | green (light) |
| Fluorescein FITC | 495 | 518 | green (light) |
| Alexa Fluor 430 | 430 | 545 | green (light) |
| Alexa Fluor 532 | 530 | 555 | green (light) |
| HEX | 535 | 556 | green (light) |
| Cy3 | 550 | 570 | yellow |
| TRITC | 547 | 572 | yellow |
| Alexa Fluor 546 | 556 | 573 | yellow |
| Alexa Fluor 555 | 556 | 573 | yellow |
| R-phycoerythrin (PE) | 480; 565 | 578 | yellow |
| Rhodamine Red-X | 560 | 580 | orange |
| Tamara | 565 | 580 | red |
| Cy3.5 581 | 581 | 596 | red |
| Rox | 575 | 602 | red |
| Alexa Fluor 568 | 578 | 603 | red |
| Red 613 | 480; 565 | 613 | red |
| Texas Red | 615 | 615 | red |
| Alexa Fluor 594 | 590 | 617 | red |
| Alexa Fluor 633 | 621 | 639 | red |
| Allophycocyanin | 650 | 660 | red |
| Alexa Fluor 633 | 650 | 668 | red |
| Cy5 | 650 | 670 | red |
| Alexa Fluor 660 | 663 | 690 | red |
| Cy5.5 | 675 | 694 | red |
| TruRed | 490; 675 | 695 | red |
| Alexa Fluor 680 | 679 | 702 | red |
| Cy7 | 743 | 770 | red |

Exemplary nanobody sequences are provided in Table 3 and Table 4.

In some embodiments, the nanobody bind to a target antigen. In some embodiments, the target antigen is a small molecule.

In some embodiments, the small molecule is methotrexate. In some embodiments, the nanobody comprises a nucleic acid sequence that is at least 8000, at least 81%, at least 82%, at least 83%, at least 84%, at least 85%, at least 86%, at least 8700, at least 880%, at least 89, at least 900%, at least 910%, at least 920%, at least 9300, at least 9400, at least 9500, at least 9600, at least 9800, at least 9900 identical to SEQ ID NO. 1. In some embodiments, the nanobody comprises SEQ ID NO. 1.

In some embodiments, the small molecule is caffeine. In some embodiments, the nanobody comprises a nucleic acid sequence that is at least 80%, at least 81%, at least 82%, at least 83%, at least 84%, at least 85%, at least 86%, at least 8700, at least 880%, at least 89, at least 900%, at least 910%, at least 920%, at least 9300, at least 9400 at least 9500 at least 9600, at least 9800, at least 9900 identical to SEQ ID NO. 2. In some embodiments, the nanobody comprises SEQ ID NO. 2.

TABLE 3
Exemplary Nanobody Nucleic Acid Sequences

| | | SEQ |
|---|---|---|
| Target | Nanobody Nucleic | ID |
| Antigen | Acid Sequence | NO. |
| Methotrexate | TCCCAAGTACAACTGGTAGAAAGCGGT | 1 |
| (MTX) | GGTGGTCTGGTACAAGCAGGTGGTAGC | |
| | CTGCGTCTGAGCTGCGCAGCATCCCGTC | |
| | GTTCCTCCCGTTCCTGGGCTATGGCTTG | |
| | GTTCCGTCAAGCACCAGGTAAAGAACG | |
| | TGAATTTGTGGCGAAAATTAGCGGCGA | |
| | TGGCCGTCTGACCACCTATGGAGACAG | |
| | CGTAAAAGGTCGTTTCACCATCAGCCG | |
| | TGACAACGCGGAATATCTGGTGTATCT | |
| | GCAGATGGACAGCCTGAAACCAGAAGA | |
| | CACCGCAGTTTACTACTGCGCTGCTGAT | |
| | GATAACTATGTGACCGCGAGCTGGCGT | |
| | AGCGGCCCGGATTATTGGGGCCAGGGC | |
| | ACCCAAGTAACCGTAAGCAGCTAATAG | |
| Caffeine | tcccaagtacaactggtagaaagcggtg | 2 |
| | gtggtctggtacaagcaggtggtagcct | |
| | gcgtctgagctgcaccgcttccggtcgt | |
| | accggtaccatctactccatggcttggt | |
| | tccgtcaagcaccaggtaaagaacgtga | |
| | attcctggctaccgttggttggtcctcc | |
| | ggtatcacctactacatggacagcgtaa | |
| | aaggtcgtttcaccatcagccgtgacaa | |
| | aggtaaaaacaccgtatacctgcagatg | |
| | gacagcctgaaaccagaagacaccgcag | |
| | tttactactgcaccgctacccgtgctta | |
| | ctccgttggttacgactactggggtcaa | |
| | ggcacccaagtaaccgtaagcagctaa | |

In some embodiments the target antigen is a eukaryotic cell surface protein. In some embodiments, the eukaryotic cell surface protein is an immune checkpoint ligand.

In some embodiments, the immune checkpoint ligand is PD-L1. In some embodiments, the nanobody comprises an amino acid sequence that is at least 80%, at least 81%, at least 82%, at least 83%, at least 84%, at least 85%, at least 86%, at least 87%, at least 88%, at least 89, at least 90%, at least 91%, at least 92%, at least 93%, at least 94%, at least 95%, at least 96%, at least 98%, at least 99% identical to SEQ ID NO. 1. In some embodiments, the nanobody comprises SEQ ID NO. 1.

In some embodiments, the immune checkpoint ligand is CTLA-4. In some embodiments, the nanobody comprises an amino acid sequence that is at least 80%, at least 81%, at least 82%, at least 83%, at least 84%, at least 85%, at least 86%, at least 87%, at least 88%, at least 89, at least 90%, at least 91%, at least 92%, at least 93%, at least 94%, at least 95%, at least 96%, at least 98%, at least 99% identical to SEQ ID NO. 2. In some embodiments, the nanobody comprises SEQ ID NO. 2.

TABLE 4
Exemplary Nanobody Amino Acid Sequences

| | | SEQ |
|---|---|---|
| Target | Camelidae Nanobody | ID |
| Antigen | Amino Acid Sequence | NO. |
| PD-L1 | MAQVQLVETGGGLVQPGGSLRLSCTASG | 3 |
| | FTFSMHAMTWYRQAPGKQRELVAVITSH | |
| | GDRANYTDSVRGRFTISRDNTKNMVYLQ | |
| | MNSLKPEDTAVYYCNVPRYDSWGQGTQ | |
| | VTVSSGGLPETGGHHHHHH | |
| CTLA-4 | MAQVQLVESGGGLAQPGGSLRLSCAASG | 4 |
| | STISSVAVGWYRQTPGNQREWVATSSTS | |
| | STTATYADSVKGRFTISRDNAKNTIYLQ | |
| | MNSLKPEDTAVYYCKTGLTNWGRGTQVT | |
| | VSSGGLPETGGHHHHHH | |

In some embodiments, the target antigen is a viral antigen. In some embodiments, the viral antigen is a SARS-CoV-2 antigen. In some embodiments, the SARS-CoV-2 antigen is a spike glycoprotein. In some embodiments, the viral antigen is a hepatitis B antigen. In some embodiments, the hepatitis B antigen is hepatitis B surface antigen (HBsAg). In some embodiments, the hepatitis B antigen is hepatitis B e-antigen (HBeAg). In some embodiments, the hepatitis B antigen is hepatitis B core antigen (HBcAg). In some embodiments, the target antigen is an inflammatory cytokine. In some embodiments, the inflammatory cytokine is TNF-α.

In certain aspects, provided herein is a method of inhibiting virus fusion to a human cell, comprising contacting the virus with a nanobody as described herein. In some embodiments, the virus is SARS-CoV-2 virus. In some embodiments, the virus is hepatitis B virus.

In certain aspects, the nanobodies described herein can be produced in Bacillus subtilis using any method known in the art.

In certain aspects, provided herein is a method of producing the nanobody as described herein, comprising the steps of a) expressing the vector as described herein in a Bacillus subtilis strain; and b) harvesting the nanobody from the Bacillus subtilis strain or a cell culture supernatant of the Bacillus subtilis strain. In some embodiments, the nanobody described herein comprises an affinity tag. In some embodiments, the method further comprises isolating the nanobody on an immobile substrate by binding of the affinity tag to the immobile substrate. In some embodiments, the immobile substrate is a cellulose substrate.

In some embodiments, the Bacillus subtilis strain secretes the nanobody. In some embodiments, the Bacillus subtilis strain is deficient in at least eight extracellular proteases. In some embodiments, the Bacillus subtilis strain are from a strain comprising at least 90% genomic, 16S and/or CRISPR sequence identity to the nucleotide sequence of the Bacillus subtilis strain WB800N (GenBank Accession No. CP032310.1). In some embodiments, the Bacillus subtilis strain are from a strain comprising at least 99% genomic, 16S and/or CRISPR sequence identity to the nucleotide sequence of the Bacillus subtilis strain WB800N (GenBank Accession No. CP032310.1). In some embodiments, the Bacillus subtilis strain are from Bacillus subtilis strain WB800N (GenBank Accession No. CP032310.1). In some embodiments, the nanobody is harvested from the cell culture supernatant of the Bacillus subtilis strain. In some embodiments, the Bacillus subtilis strain is a sporulated Bacillus subtilis strain prior to step (a). In some embodiments, the sporulated Bacillus subtilis strain is germinated into a vegetative Bacillus subtilis strain prior to step (a).

In certain aspects, provided herein is a method of detecting the presence of a target antigen in a sample comprising incubating the nanobody as described herein with the sample, wherein the nanobody comprises a detectable label. In some embodiments, the target antigen is a small molecule. In some embodiments, the small molecule is methotrexate. In some embodiments, the small molecule is caffeine. In some embodiments the target antigen is a eukaryotic cell surface protein. In some embodiments, the eukaryotic cell surface protein is an immune checkpoint ligand. In some embodiments, the immune checkpoint ligand is PD-L1. In some embodiments, the immune checkpoint ligand is CTLA-4. In some embodiments, the target antigen is a viral antigen. In some embodiments, the viral antigen is a SARS-CoV-2 antigen. In some embodiments, the SARS-CoV-2 antigen is a spike glycoprotein. In some embodiments, the viral antigen is a hepatitis B antigen. In some embodiments, the hepatitis B antigen is hepatitis B surface antigen (HBsAg). In some embodiments, the hepatitis B antigen is hepatitis B e-antigen (HBeAg). In some embodiments, the hepatitis B antigen is hepatitis B core antigen (HBcAg). In some embodiments, the target antigen is an inflammatory cytokine. In some embodiments, the inflammatory cytokine is TNF-α.

In certain aspects, provided herein is a kit for detecting a target antigen in a sample, comprising a device for collecting the sample and reagents for detecting the target antigen, wherein the reagents comprise the nanobody as described herein and wherein the nanobody comprises a detectable label. In some embodiments, the target antigen is a small molecule. In some embodiments, the small molecule is methotrexate. In some embodiments, the small molecule is caffeine. In some embodiments the target antigen is a eukaryotic cell surface protein. In some embodiments, the eukaryotic cell surface protein is an immune checkpoint ligand. In some embodiments, the immune checkpoint ligand is PD-L1. In some embodiments, the immune checkpoint ligand is CTLA-4. In some embodiments, the target antigen is a viral antigen. In some embodiments, the viral antigen is a SARS-CoV-2 antigen. In some embodiments, the SARS-CoV-2 antigen is a spike glycoprotein. In some embodiments, the viral antigen is a hepatitis B antigen. In some embodiments, the hepatitis B antigen is hepatitis B surface antigen (HBsAg). In some embodiments, the hepatitis B antigen is hepatitis B e-antigen (HBeAg). In some embodiments, the hepatitis B antigen is hepatitis B core antigen (HBcAg). In some embodiments, the target antigen is an inflammatory cytokine. In some embodiments, the inflammatory cytokine is TNF-α.

### Pharmaceutical Compositions

In certain aspects, provided herein is a pharmaceutical composition comprising a nanobody as described herein.

In certain aspects, provided herein is a pharmaceutical composition comprising a cell culture supernatant as described herein.

In certain aspects, provided herein is a pharmaceutical composition comprising a vegetative cell as described herein.

In certain aspects, provided herein is a pharmaceutical composition comprising a sporulated cell as described herein.

In certain aspects, provided herein is use of any one of the pharmaceutical compositions described herein for the preparation of a medicament for treating or prevention of a disease or disorder. In some embodiments, the disease or disorder is a gastrointestinal disease. In some embodiments, the disease or disorder is metabolic disorder. In some embodiments, the disease or disorder is an immunoinflammatory disease. In some embodiments, the disease or disorder is a cancer. In some embodiments, the disease or disorder is an infectious disease or disorder. In some embodiments, the infectious disease is a viral infection. In some embodiments viral infection is COVID-19. In some embodiments, the viral infection is hepatitis B infection. In some embodiments, use of any one of the pharmaceutical compositions described herein further comprises conjointly administering an additional therapeutic to the subject. In some embodiments, the additional therapeutic is an anti-inflammatory agent. In some embodiments, the additional therapeutic is a chemotherapeutic agent. In some embodiments, the additional therapeutic is an immunotherapy agent. In some embodiments, the immunotherapy is an immune checkpoint inhibitor. In some embodiments, the additional therapeutic is an anti-viral agent. In some embodiments, the disease or disorder is characterized by dysbiosis.

In certain aspects, provided herein is a method of treating a disease or disorder, comprising administering any one of the pharmaceutical compositions as described herein. In some embodiments, the disease or disorder is a gastrointestinal disease. In some embodiments, the disease or disorder is metabolic disorder. In some embodiments, the disease or disorder is an immunoinflammatory disease. In some embodiments, the disease or disorder is a cancer. In some embodiments, the disease or disorder is an infectious disease or disorder. In some embodiments, the infectious disease is a viral infection. In some embodiments viral infection is COVID-19. In some embodiments, the viral infection is hepatitis B infection. In some embodiments, use of any one of the pharmaceutical compositions described herein further comprises conjointly administering an additional therapeutic to the subject. In some embodiments, the additional therapeutic is an anti-inflammatory agent. In some embodiments, the additional therapeutic is a chemotherapeutic agent. In some embodiments, the additional therapeutic is an immunotherapy agent. In some embodiments, the immunotherapy is an immune checkpoint inhibitor. In some embodiments, the additional therapeutic is an anti-viral agent. In some embodiments, the disease or disorder is characterized by dysbiosis.

In certain embodiments, provided herein are pharmaceutical compositions comprising nanobodies. In some embodiments, the nanobody composition comprises nanobodies and a pharmaceutically acceptable carrier.

In some embodiments, the pharmaceutical compositions comprise nanobodies substantially or entirely free of whole Bacillus subtilis bacteria (e.g., live bacteria, killed bacteria, attenuated bacteria). In some embodiments, the pharmaceutical compositions comprise both nanobodies and whole Bacillus subtilis bacteria (e.g., live bacteria, killed bacteria, attenuated bacteria). In some embodiments, the pharmaceutical compositions comprise nanobodies from the Bacillus subtilis bacteria strain of Table 1. In some embodiments, the pharmaceutical composition comprises lyophilized nanobodies.

In some embodiments, the nanobodies may be quantified based on the amount of protein. For example, total protein content can be measured using the Bradford assay.

In some embodiments, the nanobodies are isolated away from one or more other bacterial components of the source bacteria. In some embodiments, the pharmaceutical composition further comprises other bacterial components.

In certain embodiments, the nanobodies preparation obtained from the source bacteria may be fractionated into subpopulations based on the physical properties (e.g., sized, density, protein content, binding affinity) of the subpopulations. One or more of the nanobody subpopulations can then be incorporated into the pharmaceutical compositions of the invention.

In certain aspects, provided herein are pharmaceutical compositions comprising nanobodies useful for the treatment and/or prevention of disease (e.g., a cancer, an autoimmune disease, infectious disease, an inflammatory disease, or a metabolic disease), as well as methods of producing such nanobodies, and methods of using such pharmaceutical compositions (e.g., for the treatment of a cancer, an autoimmune disease, infectious disease, an inflammatory disease, or a metabolic disease, either alone or in combination with other therapeutics). In some embodiments, the pharmaceutical compositions comprise both nanobodies and whole Bacillus subtilis bacteria (e.g., live bacteria, killed bacteria, attenuated bacteria). In some embodiments, the pharmaceutical compositions comprise nanobodies in the absence of bacteria. In some embodiments, the pharmaceutical compositions comprise nanobodies and/or bacteria from the Bacillus subtilis bacteria strain or species listed in Table 1

In certain aspects, provided are pharmaceutical compositions for administration to a subject (e.g., human subject). In some embodiments, the pharmaceutical compositions are combined with additional active and/or inactive materials in order to produce a final product, which may be in single dosage unit or in a multi-dose format. In some embodiments, the pharmaceutical composition is combined with an adjuvant such as an immuno-adjuvant (e.g., a STING agonist, a TLR agonist, or a NOD agonist).

In some embodiments, the pharmaceutical composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments, the excipient is a buffering agent. Non-limiting examples of suitable buffering agents include sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the pharmaceutical composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, the pharmaceutical composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the pharmaceutical composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments, the pharmaceutical composition comprises a disintegrant as an excipient. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments the disintegrant is an effervescent disintegrant. Non-limiting examples of suitable effervescent disintegrants include sodium bicarbonate in combination with citric acid, and sodium bicarbonate in combination with tartaric acid.

In some embodiments, the pharmaceutical composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In some embodiments, the pharmaceutical composition is a food product for animals, including humans. The animals, other than humans, are not particularly limited, and the composition can be used for various livestock, poultry, pets, experimental animals, and the like. Specific examples of the animals include pigs, cattle, horses, sheep, goats, chickens, wild ducks, ostriches, domestic ducks, dogs, cats, rabbits, hamsters, mice, rats, monkeys, and the like, but the animals are not limited thereto. A pharmaceutical composition comprising nanobodies can be formulated as a solid dose form, e.g., for oral administration. The solid dose form can comprise one or more excipients, e.g., pharmaceutically acceptable excipients. The nanobodies in the solid dose form can be isolated nanobodies. Optionally, the nanobodies in the solid dose form can be lyophilized. The solid dose form can comprise a tablet, a minitablet, a capsule, a pill, or a powder; or a combination of these forms (e.g., minitablets comprised in a capsule).

The solid dose form can comprise a tablet (e.g., >4 mm).

The solid dose form can comprise a mini tablet (e.g., 1-4 mm sized minitablet, e.g., a 2 mm minitablet or a 3 mm minitablet).

The solid dose form can comprise a capsule, e.g., a size 00, size 0, size 1, size 2, size 3, size 4, or size 5 capsule; e.g., a size 0 capsule.

The solid dose form can comprise a coating. The solid dose form can comprise a single layer coating, e.g., enteric coating, e.g., a Eudragit-based coating, e.g., EUDRAGIT L30 D-55, triethylcitrate, and talc. The solid dose form can comprise two layers of coating. For example, an inner coating can comprise, e.g., EUDRAGIT L30 D-55, triethylcitrate, talc, citric acid anhydrous, and sodium hydroxide, and an outer coating can comprise, e.g., EUDRAGIT L30 D-55, triethylcitrate, and talc. EUDRAGIT is the brand name for a diverse range of polymethacrylate-based copolymers. It includes anionic, cationic, and neutral copolymers based on methacrylic acid and methacrylic/acrylic esters or their derivatives. Eudragits are amorphous polymers having glass transition temperatures between 9 to >150° C. Eudragits are non-biodegradable, nonabsorbable, and nontoxic. Anionic Eudragit L dissolves at pH>6 and is used for enteric coating, while Eudragit S, soluble at pH>7 is used for colon targeting. Eudragit RL and RS, having quaternary ammonium groups, are water insoluble, but swellable/permeable polymers which are suitable for the sustained release film coating applications. Cationic Eudragit E, insoluble at pH≥5, can prevent drug release in saliva.

The solid dose form (e.g., a capsule) can comprise a single layer coating, e.g., a non-enteric coating such as HPMC (hydroxyl propyl methyl cellulose) or gelatin.

A pharmaceutical composition comprising nanobodies can be formulated as a suspension, e.g., for oral administration or for injection. Administration by injection includes intravenous (IV), intramuscular (IM), intratumoral (IT) and subcutaneous (SC) administration. For a suspension, nanobodies can be in a buffer, e.g., a pharmaceutically acceptable buffer, e.g., saline or PBS. The suspension can comprise one or more excipients, e.g., pharmaceutically acceptable excipients. The suspension can comprise, e.g., sucrose or glucose. The nanobodies in the suspension can be isolated mEVs. Optionally, the nanobodies in the suspension can be lyophilized.

### Additional Therapeutic Agents

In certain aspects, the methods provided herein include the administration to a subject of a pharmaceutical composition described herein either alone or in combination with an additional therapeutic agent. In some embodiments, the additional therapeutic agent is an immunosuppressant, anti-viral agent, an anti-inflammatory agent, a steroid, and/or a cancer therapeutic.

In some embodiments, the pharmaceutical composition comprising nanobodies is administered to the subject before the additional therapeutic agent is administered (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, the pharmaceutical composition comprising nanobodies is administered to the subject after the additional therapeutic agent is administered (e.g., at least 1, 2, 3, 4, 5, 6, 7, 8, 9, 10, 11, 12, 13, 14, 15, 16, 17, 18, 19, 20, 21, 22, 23 or 24 hours after or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the pharmaceutical composition comprising nanobodies and the additional therapeutic agent are administered to the subject simultaneously or nearly simultaneously (e.g., administrations occur within an hour of each other).

In some embodiments, an antibiotic is administered to the subject before the pharmaceutical composition comprising nanobodies is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, an antibiotic is administered to the subject after pharmaceutical composition comprising nanobodies is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the pharmaceutical composition comprising nanobodies and the antibiotic are administered to the subject simultaneously or nearly simultaneously (e.g., administrations occur within an hour of each other).

In some embodiments, the additional therapeutic agent is a cancer therapeutic. In some embodiments, the cancer therapeutic is a chemotherapeutic agent. Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegal1; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylomithine (DMFO); retinoids such as retinoic acid; capecitabine; and pharmaceutically acceptable salts, acids or derivatives of any of the above.

In some embodiments, the cancer therapeutic is a cancer immunotherapy agent. Immunotherapy refers to a treatment that uses a subject's immune system to treat cancer, e.g., checkpoint inhibitors, cancer vaccines, cytokines, cell therapy, CAR-T cells, and dendritic cell therapy. Non-limiting examples of immunotherapies are checkpoint inhibitors include Nivolumab (BMS, anti-PD-1), Pembrolizumab (Merck, anti-PD-1), Ipilimumab (BMS, anti-CTLA-4), MEDI4736 (AstraZeneca, anti-PD-L1), and MPDL3280A (Roche, anti-PD-L1). Other immunotherapies may be tumor vaccines, such as Gardail, Cervarix, BCG, sipulencel-T, Gp100:209-217, AGS-003, DCVax-L, Algenpantucel-L, Tergenpantucel-L, TG4010, ProstAtak, Prostvac-V/R-TRICOM, Rindopepimul, E75 peptide acetate, IMA901, POL-103A, Belagenpumatucel-L, GSK1572932A, MDX-1279, GV1001, and Tecemotide. The immunotherapy agent may be administered via injection (e.g., intravenously, intratumorally, subcutaneously, or into lymph nodes), but may also be administered orally, topically, or via aerosol. Immunotherapies may comprise adjuvants such as cytokines.

In some embodiments, the immunotherapy agent is an immune checkpoint inhibitor. Immune checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, MEDI-4736, MSB-0020718C, AUR-012 and STI-A1010.

In some embodiments, the additional therapeutic agent is an antiviral agent. Antiviral agents are pharmaceutical agents that inhibit viral growth. Such agents may include, but are not limited to, penciclovir, acyclovir, famciclovir, valacyclovir, tenofovir disoproxil fumarate, lamivudine, zidovudine, didanosine, emtricitabine, stavudine, nevirapine, abacavir, raltegravir, dolutegravir, darunavir, ritonavir, cobicistat, efavirenz, ribavirin, neuraminidase inhibitor, recombinant interferons, recombinant immunoglobulins, oseltamivir, zanamivir, peramivir, baloxavir marboxil, remdesivir (RDV), tilorone, favipiravir, IFN-alpha, IFN-beta, IFN-gamma, IFN-lambda, peginterferon-alpha, peginterferon-beta, ribavirin, lopinavir/ritonavir, TAK888, adefovir, amantadine, rintatolimod (Ampligen), amprenavir, umifenovir (Arbidol), atazanavir, and ivermectin. Therapeutically effective amounts for treatment are familiar to those skilled in the art.

In some embodiments, the methods provided herein include the administration of a pharmaceutical composition described herein in combination with one or more additional therapeutic agents. In some embodiments, the methods disclosed herein include the administration of two immunotherapy agents (e.g., immune checkpoint inhibitor). For example, the methods provided herein include the administration of a pharmaceutical composition described herein in combination with a PD-1 inhibitor (such as pemrolizumab or nivolumab or pidilizumab) or a CLTA-4 inhibitor (such as ipilimumab) or a PD-L1 inhibitor.

In some embodiments, the immunotherapy agent is an antibody or antigen binding fragment thereof that, for example, binds to a cancer-associated antigen. Examples of cancer-associated antigens include, but are not limited to, adipophilin, AIM-2, ALDHIAI, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pmel17, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secernin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-1b/GAGED2a. In some embodiments, the antigen is a neo-antigen.

In some embodiments, the immunotherapy agent is a cancer vaccine and/or a component of a cancer vaccine (e.g., an antigenic peptide and/or protein). The cancer vaccine can be a protein vaccine, a nucleic acid vaccine or a combination thereof. For example, in some embodiments, the cancer vaccine comprises a polypeptide comprising an epitope of a cancer-associated antigen. In some embodiments, the cancer vaccine comprises a nucleic acid (e.g., DNA or RNA, such as mRNA) that encodes an epitope of a cancer-associated antigen. Examples of cancer-associated antigens include, but are not limited to, adipophilin, AIM-2, ALDHIAI, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (“CEA”), CASP-5, CASP-8, CD274, CD45, Cdc27, CDK12, CDK4, CDKN2A, CEA, CLPP, COA-1, CPSF, CSNK1A1, CTAG1, CTAG2, cyclin D1, Cyclin-A1, dek-can fusion protein, DKK1, EFTUD2, Elongation factor 2, ENAH (hMena), Ep-CAM, EpCAM, EphA3, epithelial tumor antigen (“ETA”), ETV6-AML1 fusion protein, EZH2, FGF5, FLT3-ITD, FN1, G250/MN/CAIX, GAGE-1,2,8, GAGE-3,4,5,6,7, GAS7, glypican-3, GnTV, gp100/Pmel17, GPNMB, HAUS3, Hepsin, HER-2/neu, HERV-K-MEL, HLA-A11, HLA-A2, HLA-DOB, hsp70-2, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDC110, LAGE-1, LDLR-fucosyltransferaseAS fusion protein, Lengsin, M-CSF, MAGE-A1, MAGE-A10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-1/LAGE-2, OA1, OGT, OS-9, P polypeptide, p53, PAP, PAX5, PBF, pml-RARalpha fusion protein, polymorphic epithelial mucin (“PEM”), PPP1R3B, PRAME, PRDX5, PSA, PSMA, PTPRK, RAB38/NY-MEL-1, RAGE-1, RBAF600, RGS5, RhoC, RNF43, RU2AS, SAGE, secernin 1, SIRT2, SNRPD1, SOX10, Sp17, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-1/gp75, TRP-2, TRP2-INT2, tyrosinase, tyrosinase (“TYR”), VEGF, WT1, XAGE-1b/GAGED2a. In some embodiments, the antigen is a neo-antigen. In some embodiments, the cancer vaccine is administered with an adjuvant. Examples of adjuvants include, but are not limited to, an immune modulatory protein, Adjuvant 65, α-GalCer, aluminum phosphate, aluminum hydroxide, calcium phosphate, β-Glucan Peptide, CpG ODN DNA, GPI-0100, lipid A, lipopolysaccharide, Lipovant, Montanide, N-acetyl-muramyl-L-alanyl-D-isoglutamine, Pam3CSK4, quil A, cholera toxin (CT) and heat-labile toxin from enterotoxigenic Escherichia coli (LT) including derivatives of these (CTB, mmCT, CTA1-DD, LTB, LTK63, LTR72, dmLT) and trehalose dimycolate.

In some embodiments, the immunotherapy agent is an immune modulating protein to the subject. In some embodiments, the immune modulatory protein is a cytokine or chemokine. Examples of immune modulating proteins include, but are not limited to, B lymphocyte chemoattractant (“BLC”), C-C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon alpha (“IFN-alpha”), Interferon beta (“IFN-beta”) Interferon gamma (“IFN-gamma”), Interleukin-1 alpha (“IL-1 alpha”), Interleukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17A-F (“IL-17A-F”), Interleukin-18 (“IL-18”), Interleukin-21 (“IL-21”), Interleukin-22 (“IL-22”), Interleukin-23 (“IL-23”), Interleukin-33 (“IL-33”), Chemokine (C-C motif) Ligand 2 (“MCP-1”), Macrophage colony-stimulating factor (“M-CSF”), Monokine induced by gamma interferon (“MIG”), Chemokine (C-C motif) ligand 2 (“MIP-1 alpha”), Chemokine (C-C motif) ligand 4 (“MIP-1 beta”), Macrophage inflammatory protein-1-delta (“MIP-1 delta”), Platelet-derived growth factor subunit B (“PDGF-BB”), Chemokine (C-C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing comples (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C-C motif) ligand 27 (“CTACK”), Chemokine (C-X-C motif) ligand 16 (“CXCL16”), C-X-C motif chemokine 5 (“ENA-78”), Chemokine (C-C motif) ligand 26 (“Eotaxin-3”), Granulocyte chemotactic protein 2 (“GCP-2”), GRO, Chemokine (C-C motif) ligand 14 (“HCC-1”), Chemokine (C-C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C-X-C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C-C motif) ligand 20 (“MIP-3 alpha”), C-C motif chemokine 19 (“MIP-3 beta”), Chemokine (C-C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C-C motif) ligand 17 (“TARC”), Thymus-expressed chemokine (“TECK”), Thymic stromal lymphopoietin (“TSLP 4-IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRGl-betal, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDARActivin A, Agouti-related protein (“AgRP”), Ribonuclease 5 (“Angiogenin”), Angiopoietin 1, Angiostatin, Catheprin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, Follistatin, Galectin-7, Intercellular adhesion molecule 2 (“ICAM-2”), IL-13 μl, IL-13R2, IL-17B, IL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgpl30, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C-X-C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-gammalpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Tolllike receptor 2 (“TLR2”), Tumor necrosis factor receptor superfamily member 10A (“TRAIL Rl”), Transferrin (“TRF”), WIF-IACE-2, Albumin, AMICA, Angiopoietin 4, B-cell activating factor (“BAFF”), Carbohydrate antigen 19-9 (“CA19-9”), CD 163, Clusterin, CRT AM, Chemokine (C-X-C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNTI-inducible-signaling pathway protein 1 (“WISP-1”), and Receptor Activator of Nuclear Factor κ B (“RANK”).

In some embodiments, the cancer therapeutic is an anti-cancer compound. Exemplary anti-cancer compounds include, but are not limited to, Alemtuzumab (Campath®), Alitretinoin (Panretin®), Anastrozole (Arimidex®), Bevacizumab (Avastin®), Bexarotene (Targretin®), Bortezomib (Velcade®), Bosutinib (Bosulif®), Brentuximab vedotin (Adcetris®), Cabozantinib (Cometriq™), Carfilzomib (Kyprolis™) Cetuximab (Erbitux®), Crizotinib (Xalkori®), Dasatinib (Sprycel®), Denileukin diftitox (Ontak®), Erlotinib hydrochloride (Tarceva®), Everolimus (Afinitor®), Exemestane (Aromasin®), Fulvestrant (Faslodex®), Gefitinib (Iressa®), Ibritumomab tiuxetan (Zevalin®), Imatinib mesylate (Gleevec®), Ipilimumab (Yervoy™), Lapatinib ditosylate (Tykerb®), Letrozole (Femara®), Nilotinib (Tasigna®), Ofatumumab (Arzerra®), Panitumumab (Vectibix®), Pazopanib hydrochloride (Votrient®), Pertuzumab (Perjeta™), Pralatrexate (Folotyn®), Regorafenib (Stivarga®), Rituximab (Rituxan®), Romidepsin (Istodax®), Sorafenib tosylate (Nexavar®), Sunitinib malate (Sutent®), Tamoxifen, Temsirolimus (Torisel®), Toremifene (Fareston®), Tositumomab and 131I-tositumomab (Bexxar®), Trastuzumab (Herceptin®), Tretinoin (Vesanoid®), Vandetanib (Caprelsa®), Vemurafenib (Zelboraf®), Vorinostat (Zolinza®), and Ziv-aflibercept (Zaltrap®).

Exemplary anti-cancer compounds that modify the function of proteins that regulate gene expression and other cellular functions (e.g., HDAC inhibitors, retinoid receptor ligants) are Vorinostat (Zolinza®), Bexarotene (Targretin®) and Romidepsin (Istodax®), Alitretinoin (Panretin®), and Tretinoin (Vesanoid®).

Exemplary anti-cancer compounds that induce apoptosis (e.g., proteasome inhibitors, antifolates) are Bortezomib (Velcade®), Carfilzomib (Kyprolis™), and Pralatrexate (Folotyn®).

Exemplary anti-cancer compounds that increase anti-tumor immune response (e.g., anti CD20, anti CD52; anti-cytotoxic T-lymphocyte-associated antigen-4) are Rituximab (Rituxan®), Alemtuzumab (Campath®), Ofatumumab (Arzerra®), and Ipilimumab (Yervoy™)

Exemplary anti-cancer compounds that deliver toxic agents to cancer cells (e.g., anti-CD20-radionuclide fusions; IL-2-diphtheria toxin fusions; anti-CD30-monomethylauristatin E (MMAE)-fusions) are Tositumomab and 131I-tositumomab (Bexxar®) and Ibritumomab tiuxetan (Zevalin®), Denileukin diftitox (Ontak®), and Brentuximab vedotin (Adcetris®).

Other exemplary anti-cancer compounds are small molecule inhibitors and conjugates thereof of, e.g., Janus kinase, ALK, Bcl-2, PARP, PI3K, VEGF receptor, Braf, MEK, CDK, and HSP90.

Exemplary platinum-based anti-cancer compounds include, for example, cisplatin, carboplatin, oxaliplatin, satraplatin, picoplatin, Nedaplatin, Triplatin, and Lipoplatin. Other metal-based drugs suitable for treatment include, but are not limited to ruthenium-based compounds, ferrocene derivatives, titanium-based compounds, and gallium-based compounds.

In some embodiments, the cancer therapeutic is a radioactive moiety that comprises a radionuclide. Exemplary radionuclides include, but are not limited to Cr-51, Cs-131, Ce-134, Se-75, Ru-97, I-125, Eu-149, Os-189m, Sb-119, I-123, Ho-161, Sb-117, Ce-139, In-111, Rh-103m, Ga-67, T1-201, Pd-103, Au-195, Hg-197, Sr-87m, Pt-191, P-33, Er-169, Ru-103, Yb-169, Au-199, Sn-121, Tm-167, Yb-175, In-113m, Sn-113, Lu-177, Rh-105, Sn-117m, Cu-67, Sc-47, Pt-195m, Ce-141, I-131, Tb-161, As-77, Pt-197, Sm-153, Gd-159, Tm-173, Pr-143, Au-198, Tm-170, Re-186, Ag-111, Pd-109, Ga-73, Dy-165, Pm-149, Sn-123, Sr-89, Ho-166, P-32, Re-188, Pr-142, Ir-194, In-114m/In-114, and Y-90.

In some embodiments, the cancer therapeutic is an antibiotic. For example, if the presence of a cancer-associated bacteria and/or a cancer-associated microbiome profile is detected according to the methods provided herein, antibiotics can be administered to eliminate the cancer-associated bacteria from the subject. “Antibiotics” broadly refers to compounds capable of inhibiting or preventing a bacterial infection. Antibiotics can be classified in a number of ways, including their use for specific infections, their mechanism of action, their bioavailability, or their spectrum of target microbe (e.g., Gram-negative vs. Gram-positive bacteria, aerobic vs. anaerobic bacteria, etc.) and these may be used to kill specific bacteria in specific areas of the host (“niches”) (Leekha, et al 2011. General Principles of Antimicrobial Therapy. Mayo Clin Proc. 86(2): 156-167). In certain embodiments, antibiotics can be used to selectively target bacteria of a specific niche. In some embodiments, antibiotics known to treat a particular infection that includes a cancer niche may be used to target cancer-associated microbes, including cancer-associated bacteria in that niche. In other embodiments, antibiotics are administered after the pharmaceutical composition comprising mEVsnanobodies. In some embodiments, antibiotics are administered before pharmaceutical composition comprising nanobodies

In some aspects, antibiotics can be selected based on their bactericidal or bacteriostatic properties. Bactericidal antibiotics include mechanisms of action that disrupt the cell wall (e.g., β-lactams), the cell membrane (e.g., daptomycin), or bacterial DNA (e.g., fluoroquinolones). Bacteriostatic agents inhibit bacterial replication and include sulfonamides, tetracyclines, and macrolides, and act by inhibiting protein synthesis. Furthermore, while some drugs can be bactericidal in certain organisms and bacteriostatic in others, knowing the target organism allows one skilled in the art to select an antibiotic with the appropriate properties. In certain treatment conditions, bacteriostatic antibiotics inhibit the activity of bactericidal antibiotics. Thus, in certain embodiments, bactericidal and bacteriostatic antibiotics are not combined.

Antibiotics include, but are not limited to aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, and anti-mycobacterial compounds, and combinations thereof.

Aminoglycosides include, but are not limited to Amikacin, Gentamicin, Kanamycin, Neomycin, Netilmicin, Tobramycin, Paromomycin, and Spectinomycin. Aminoglycosides are effective, e.g., against Gram-negative bacteria, such as Escherichia coli, Klebsiella, Pseudomonas aeruginosa, and Francisella tularensis, and against certain aerobic bacteria but less effective against obligate/facultative anaerobes. Aminoglycosides are believed to bind to the bacterial 30S or 50S ribosomal subunit thereby inhibiting bacterial protein synthesis.

Ansamycins include, but are not limited to, Geldanamycin, Herbimycin, Rifamycin, and Streptovaricin. Geldanamycin and Herbimycin are believed to inhibit or alter the function of Heat Shock Protein 90.

Carbacephems include, but are not limited to, Loracarbef Carbacephems are believed to inhibit bacterial cell wall synthesis.

Carbapenems include, but are not limited to, Ertapenem, Doripenem, Imipenem/Cilastatin, and Meropenem. Carbapenems are bactericidal for both Gram-positive and Gram-negative bacteria as broad-spectrum antibiotics. Carbapenems are believed to inhibit bacterial cell wall synthesis.

Cephalosporins include, but are not limited to, Cefadroxil, Cefazolin, Cefalotin, Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, and Ceftobiprole. Selected Cephalosporins are effective, e.g., against Gram-negative bacteria and against Gram-positive bacteria, including Pseudomonas, certain Cephalosporins are effective against methicillin-resistant Staphylococcus aureus (MRSA). Cephalosporins are believed to inhibit bacterial cell wall synthesis by disrupting synthesis of the peptidoglycan layer of bacterial cell walls.

Glycopeptides include, but are not limited to, Teicoplanin, Vancomycin, and Telavancin. Glycopeptides are effective, e.g., against aerobic and anaerobic Gram-positive bacteria including MRSA and Clostridium difficile. Glycopeptides are believed to inhibit bacterial cell wall synthesis by disrupting synthesis of the peptidoglycan layer of bacterial cell walls.

Lincosamides include, but are not limited to, Clindamycin and Lincomycin. Lincosamides are effective, e.g., against anaerobic bacteria, as well as Staphylococcus, and Streptococcus. Lincosamides are believed to bind to the bacterial 50S ribosomal subunit thereby inhibiting bacterial protein synthesis.

Lipopeptides include, but are not limited to, Daptomycin. Lipopeptides are effective, e.g., against Gram-positive bacteria. Lipopeptides are believed to bind to the bacterial membrane and cause rapid depolarization.

Macrolides include, but are not limited to, Azithromycin, Clarithromycin, Dirithromycin, Erythromycin, Roxithromycin, Troleandomycin, Telithromycin, and Spiramycin. Macrolides are effective, e.g., against Streptococcus and Mycoplasma. Macrolides are believed to bind to the bacterial or 50S ribosomal subunit, thereby inhibiting bacterial protein synthesis.

Monobactams include, but are not limited to, Aztreonam. Monobactams are effective, e.g., against Gram-negative bacteria. Monobactams are believed to inhibit bacterial cell wall synthesis by disrupting synthesis of the peptidoglycan layer of bacterial cell walls.

Nitrofurans include, but are not limited to, Furazolidone and Nitrofurantoin.

Oxazolidonones include, but are not limited to, Linezolid, Posizolid, Radezolid, and Torezolid. Oxazolidonones are believed to be protein synthesis inhibitors.

Penicillins include, but are not limited to, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin and Ticarcillin. Penicillins are effective, e.g., against Gram-positive bacteria, facultative anaerobes, e.g., Streptococcus, Borrelia, and Treponema. Penicillins are believed to inhibit bacterial cell wall synthesis by disrupting synthesis of the peptidoglycan layer of bacterial cell walls.

Penicillin combinations include, but are not limited to, Amoxicillin/clavulanate, Ampicillin/sulbactam, Piperacillin/tazobactam, and Ticarcillin/clavulanate.

Polypeptide antibiotics include, but are not limited to, Bacitracin, Colistin, and Polymyxin B and E. Polypeptide Antibiotics are effective, e.g., against Gram-negative bacteria. Certain polypeptide antibiotics are believed to inhibit isoprenyl pyrophosphate involved in synthesis of the peptidoglycan layer of bacterial cell walls, while others destabilize the bacterial outer membrane by displacing bacterial counter-ions.

Quinolones and Fluoroquinolone include, but are not limited to, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin. Quinolones/Fluoroquinolone are effective, e.g., against Streptococcus and Neisseria. Quinolones/Fluoroquinolone are believed to inhibit the bacterial DNA gyrase or topoisomerase IV, thereby inhibiting DNA replication and transcription.

Sulfonamides include, but are not limited to, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole), and Sulfonamidochrysoidine. Sulfonamides are believed to inhibit folate synthesis by competitive inhibition of dihydropteroate synthetase, thereby inhibiting nucleic acid synthesis.

Tetracyclines include, but are not limited to, Demeclocycline, Doxycycline, Minocycline, Oxytetracycline, and Tetracycline. Tetracyclines are effective, e.g., against Gram-negative bacteria. Tetracyclines are believed to bind to the bacterial 30S ribosomal subunit thereby inhibiting bacterial protein synthesis.

Anti-mycobacterial compounds include, but are not limited to, Clofazimine, Dapsone, Capreomycin, Cycloserine, Ethambutol, Ethionamide, Isoniazid, Pyrazinamide, Rifampicin, Rifabutin, Rifapentine, and Streptomycin.

Suitable antibiotics also include arsphenamine, chloramphenicol, fosfomycin, fusidic acid, metronidazole, mupirocin, platensimycin, quinupristin/dalfopristin, tigecycline, tinidazole, trimethoprim amoxicillin/clavulanate, ampicillin/sulbactam, amphomycin ristocetin, azithromycin, bacitracin, buforin II, carbomycin, cecropin Pl, clarithromycin, erythromycins, furazolidone, fusidic acid, Na fusidate, gramicidin, imipenem, indolicidin, josamycin, magainan II, metronidazole, nitroimidazoles, mikamycin, mutacin B-Ny266, mutacin B-JHl 140, mutacin J-T8, nisin, nisin A, novobiocin, oleandomycin, ostreogrycin, piperacillin/tazobactam, pristinamycin, ramoplanin, ranalexin, reuterin, rifaximin, rosamicin, rosaramicin, spectinomycin, spiramycin, staphylomycin, streptogramin, streptogramin A, synergistin, taurolidine, teicoplanin, telithromycin, ticarcillin/clavulanic acid, triacetyloleandomycin, tylosin, tyrocidin, tyrothricin, vancomycin, vemamycin, and virginiamycin.

In some embodiments, the additional therapeutic agent is an immunosuppressive agent, a DMARD, a pain-control drug, a steroid, a non-steroidal antiinflammatory drug (NSAID), or a cytokine antagonist, and combinations thereof. Representative agents include, but are not limited to, cyclosporin, retinoids, corticosteroids, propionic acid derivative, acetic acid derivative, enolic acid derivatives, fenamic acid derivatives, Cox-2 inhibitors, lumiracoxib, ibuprophen, cholin magnesium salicylate, fenoprofen, salsalate, difunisal, tolmetin, ketoprofen, flurbiprofen, oxaprozin, indomethacin, sulindac, etodolac, ketorolac, nabumetone, naproxen, valdecoxib, etoricoxib, MK0966; rofecoxib, acetominophen, Celecoxib, Diclofenac, tramadol, piroxicam, meloxicam, tenoxicam, droxicam, lornoxicam, isoxicam, mefanamic acid, meclofenamic acid, flufenamic acid, tolfenamic, valdecoxib, parecoxib, etodolac, indomethacin, aspirin, ibuprophen, firocoxib, methotrexate (MTX), antimalarial drugs (e.g., hydroxychloroquine and chloroquine), sulfasalazine, Leflunomide, azathioprine, cyclosporin, gold salts, minocycline, cyclophosphamide, D-penicillamine, minocycline, auranofin, tacrolimus, myocrisin, chlorambucil, TNF alpha antagonists (e.g., TNF alpha antagonists or TNF alpha receptor antagonists), e.g., ADALIMUMAB (Humira®), ETANERCEPT (Enbrel@), INFLIXIMAB (Remicade®; TA-650), CERTOLIZUMAB PEGOL (Cimzia®; CDP870), GOLIMUMAB (Simpom®; CNTO 148), ANAKINRA (Kineret®), RITUXIMAB (Rituxan®; MabThera®), ABATACEPT (Orencia®), TOCILIZUMAB (RoActemra/Actemra®), integrin antagonists (TYSABRI® (natalizumab)), IL-1 antagonists (ACZ885 (Ilaris)), Anakinra (Kineret®)), CD4 antagonists, IL-23 antagonists, IL-20 antagonists, IL-6 antagonists, BLyS antagonists (e.g., Atacicept, Benlysta®/LymphoStat-B® (belimumab)), p38 Inhibitors, CD20 antagonists (Ocrelizumab, Ofatumumab (Arzerra®)), interferon gamma antagonists (Fontolizumab), prednisolone, Prednisone, dexamethasone, Cortisol, cortisone, hydrocortisone, methylprednisolone, betamethasone, triamcinolone, beclometasome, fludrocortisone, deoxycorticosterone, aldosterone, Doxycycline, vancomycin, pioglitazone, SBI-087, SCIO-469, Cura-100, Oncoxin+Viusid, TwHF, Methoxsalen, Vitamin D—ergocalciferol, Milnacipran, Paclitaxel, rosig tazone, Tacrolimus (Prograf®), RADOO1, rapamune, rapamycin, fostamatinib, Fentanyl, XOMA 052, Fostamatinib disodium, rosightazone, Curcumin (Longvida™), Rosuvastatin, Maraviroc, ramipnl, Milnacipran, Cobiprostone, somatropin, tgAAC94 gene therapy vector, MK0359, GW856553, esomeprazole, everolimus, trastuzumab, JAKI and JAK2 inhibitors, pan JAK inhibitors, e.g., tetracyclic pyridone 6 (P6), 325, PF-956980, denosumab, IL-6 antagonists, CD20 antagonistis, CTLA4 antagonists, IL-8 antagonists, IL-21 antagonists, IL-22 antagonist, integrin antagonists (Tysarbri® (natalizumab)), VGEF antagnosits, CXCL antagonists, MMP antagonists, defensin antagonists, IL-1 antagonists (including IL-1 beta antagonsits), and IL-23 antagonists (e.g., receptor decoys, antagonistic antibodies, etc.).

In some embodiments, the additional therapeutic agent is an immunosuppressive agent. Examples of immunosuppressive agents include, but are not limited to, corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, TLR antagonists, inflammasome inhibitors, anti-cholinergic decongestants, mast-cell stabilizers, monoclonal anti-IgE antibodies, vaccines (e.g., vaccines used for vaccination where the amount of an allergen is gradually increased), cytokine inhibitors, such as anti-IL-6 antibodies, TNF inhibitors such as infliximab, adalimumab, certolizumab pegol, golimumab, or etanercept, and combinations thereof.

Administration

In certain aspects, provided herein is a method of delivering a pharmaceutical composition described herein (e.g., a pharmaceutical composition comprising nanobodies to a subject. In some embodiments of the methods provided herein, the pharmaceutical composition is administered in conjunction with the administration of an additional therapeutic agent. In some embodiments, the pharmaceutical composition comprises nanobodies co-formulated with the additional therapeutic agent. In some embodiments, the pharmaceutical composition comprising nanobodies is co-administered with the additional therapeutic agent. In some embodiments, the additional therapeutic agent is administered to the subject before administration of the pharmaceutical composition that comprises nanobodies (e.g., about 1, 2, 3, 4, 5, 6, 7, 8, 9, 10, 15, 20, 25, 30, 35, 40, 45, 50 or 55 minutes before, about 1, 2, 3, 4, 5, 6, 7, 8, 9, 10, 11, 12, 13, 14, 15, 16, 17, 18, 19, 20, 21, 22 or 23 hours before, or about 1, 2, 3, 4, 5, 6, 7, 8, 9, 10, 11, 12, 13 or 14 days before). In some embodiments, the additional therapeutic agent is administered to the subject after administration of the pharmaceutical composition that comprises nanobodies (e.g., about 1, 2, 3, 4, 5, 6, 7, 8, 9, 10, 15, 20, 25, 30, 35, 40, 45, 50 or 55 minutes after, about 1, 2, 3, 4, 5, 6, 7, 8, 9, 10, 11, 12, 13, 14, 15, 16, 17, 18, 19, 20, 21, 22 or 23 hours after, or about 1, 2, 3, 4, 5, 6, 7, 8, 9, 10, 11, 12, 13 or 14 days after). In some embodiments, the same mode of delivery is used to deliver both the pharmaceutical composition that comprises nanobodies and the additional therapeutic agent. In some embodiments, different modes of delivery are used to administer the pharmaceutical composition that comprises nanobodies and the additional therapeutic agent. For example, in some embodiments the pharmaceutical composition that comprises nanobodies is administered orally while the additional therapeutic agent is administered via injection (e.g., an intravenous, intramuscular and/or intratumoral injection).

In some embodiments, the pharmaceutical composition described herein is administered once a day. In some embodiments, the pharmaceutical composition described herein is administered twice a day. In some embodiments, the pharmaceutical composition described herein is formulated for a daily dose. In some embodiments, the pharmaceutical composition described herein is formulated for twice a day dose, wherein each dose is half of the daily dose.

In certain embodiments, the pharmaceutical compositions and dosage forms described herein can be administered in conjunction with any other conventional anti-cancer treatment, such as, for example, radiation therapy and surgical resection of the tumor. These treatments may be applied as necessary and/or as indicated and may occur before, concurrent with or after administration of the pharmaceutical composition that comprises nanobodies or dosage forms described herein.

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular microorganism to be administered, duration and route of administration, the kind and stage of the disease, for example, tumor size, and other compounds such as drugs being administered concurrently or near-concurrently. In addition to the above factors, such levels can be affected by the infectivity of the microorganism, and the nature of the microorganism, as can be determined by one skilled in the art. In the present methods, appropriate minimum dosage levels of microorganisms can be levels sufficient for the microorganism to survive, grow and replicate. The dose of a pharmaceutical composition that comprises nanobodies described herein may be appropriately set or adjusted in accordance with the dosage form, the route of administration, the degree or stage of a target disease, and the like. For example, the general effective dose of the agents may range between 0.01 mg/kg body weight/day and 1000 mg/kg body weight/day, between 0.1 mg/kg body weight/day and 1000 mg/kg body weight/day, 0.5 mg/kg body weight/day and 500 mg/kg body weight/day, 1 mg/kg body weight/day and 100 mg/kg body weight/day, or between 5 mg/kg body weight/day and 50 mg/kg body weight/day. The effective dose may be 0.01, 0.05, 0.1, 0.5, 1, 2, 3, 5, 10, 20, 30, 40, 50, 60, 70, 80, 90, 100, 200, 500, or 1000 mg/kg body weight/day or more, but the dose is not limited thereto.

In some embodiments, the dose administered to a subject is sufficient to prevent disease (e.g., autoimmune disease, infectious disease, inflammatory disease, metabolic disease, or cancer), delay its onset, or slow or stop its progression, or relieve one or more symptoms of the disease. One skilled in the art will recognize that dosage will depend upon a variety of factors including the strength of the particular agent (e.g., therapeutic agent) employed, as well as the age, species, condition, and body weight of the subject. The size of the dose will also be determined by the route, timing, and frequency of administration as well as the existence, nature, and extent of any adverse side-effects that might accompany the administration of a particular therapeutic agent and the desired physiological effect.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. An effective dosage and treatment protocol can be determined by routine and conventional means, starting e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications, the dosages of the therapeutic agents used in accordance with the invention vary depending on the active agent, the age, weight, and clinical condition of the recipient patient, and the experience and judgment of the clinician or practitioner administering the therapy, among other factors affecting the selected dosage. For example, for cancer treatment, the dose should be sufficient to result in slowing, and preferably regressing, the growth of a tumor and most preferably causing complete regression of the cancer, or reduction in the size or number of metastases As another example, the dose should be sufficient to result in slowing of progression of the disease for which the subject is being treated, and preferably amelioration of one or more symptoms of the disease for which the subject is being treated.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a pharmaceutical composition, where the number of administrations can be determined by monitoring the subject, and, based on the results of the monitoring, determining whether or not to provide one or more additional administrations. Deciding on whether or not to provide one or more additional administrations can be based on a variety of monitoring results.

The time period between administrations can be any of a variety of time periods. The time period between administrations can be a function of any of a variety of factors, including monitoring steps, as described in relation to the number of administrations, the time period for a subject to mount an immune response. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month.

In some embodiments, the delivery of an additional therapeutic agent in combination with the pharmaceutical composition described herein reduces the adverse effects and/or improves the efficacy of the additional therapeutic agent.

The effective dose of an additional therapeutic agent described herein is the amount of the additional therapeutic agent that is effective to achieve the desired therapeutic response for a particular subject, composition, and mode of administration, with the least toxicity to the subject. The effective dosage level can be identified using the methods described herein and will depend upon a variety of pharmacokinetic factors including the activity of the particular compositions or agents administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors well known in the medical arts. In general, an effective dose of an additional therapeutic agent will be the amount of the additional therapeutic agent which is the lowest dose effective to produce a therapeutic effect. Such an effective dose will generally depend upon the factors described above.

The toxicity of an additional therapeutic agent is the level of adverse effects experienced by the subject during and following treatment. Adverse events associated with additional therapy toxicity can include, but are not limited to, abdominal pain, acid indigestion, acid reflux, allergic reactions, alopecia, anaphylasix, anemia, anxiety, lack of appetite, arthralgias, asthenia, ataxia, azotemia, loss of balance, bone pain, bleeding, blood clots, low blood pressure, elevated blood pressure, difficulty breathing, bronchitis, bruising, low white blood cell count, low red blood cell count, low platelet count, cardiotoxicity, cystitis, hemorrhagic cystitis, arrhythmias, heart valve disease, cardiomyopathy, coronary artery disease, cataracts, central neurotoxicity, cognitive impairment, confusion, conjunctivitis, constipation, coughing, cramping, cystitis, deep vein thrombosis, dehydration, depression, diarrhea, dizziness, dry mouth, dry skin, dyspepsia, dyspnea, edema, electrolyte imbalance, esophagitis, fatigue, loss of fertility, fever, flatulence, flushing, gastric reflux, gastroesophageal reflux disease, genital pain, granulocytopenia, gynecomastia, glaucoma, hair loss, hand-foot syndrome, headache, hearing loss, heart failure, heart palpitations, heartburn, hematoma, hemorrhagic cystitis, hepatotoxicity, hyperamylasemia, hypercalcemia, hyperchloremia, hyperglycemia, hyperkalemia, hyperlipasemia, hypermagnesemia, hypernatremia, hyperphosphatemia, hyperpigmentation, hypertriglyceridemia, hyperuricemia, hypoalbuminemia, hypocalcemia, hypochloremia, hypoglycemia, hypokalemia, hypomagnesemia, hyponatremia, hypophosphatemia, impotence, infection, injection site reactions, insomnia, iron deficiency, itching, joint pain, kidney failure, leukopenia, liver dysfunction, memory loss, menopause, mouth sores, mucositis, muscle pain, myalgias, myelosuppression, myocarditis, neutropenic fever, nausea, nephrotoxicity, neutropenia, nosebleeds, numbness, ototoxicity, pain, palmar-plantar erythrodysesthesia, pancytopenia, pericarditis, peripheral neuropathy, pharyngitis, photophobia, photosensitivity, pneumonia, pneumonitis, proteinuria, pulmonary embolus, pulmonary fibrosis, pulmonary toxicity, rash, rapid heart beat, rectal bleeding, restlessness, rhinitis, seizures, shortness of breath, sinusitis, thrombocytopenia, tinnitus, urinary tract infection, vaginal bleeding, vaginal dryness, vertigo, water retention, weakness, weight loss, weight gain, and xerostomia. In general, toxicity is acceptable if the benefits to the subject achieved through the therapy outweigh the adverse events experienced by the subject due to the therapy.

### Immunoinflammatory Disorders

In some embodiments, the methods and compositions described herein relate to the treatment or prevention of a disease or disorder associated a pathological immune response, such as an autoimmune disease, an allergic reaction and/or an inflammatory disease. In some embodiments, the disease or disorder is an inflammatory bowel disease (e.g., Crohn's disease or ulcerative colitis). In some embodiments, the disease or disorder is psoriasis (e.g., mild to moderate psoriasis). In some embodiments, the disease or disorder is atopic dermatitis (e.g., mild to moderate atopic dermatitis).

The methods described herein can be used to treat any subject in need thereof. As used herein, a “subject in need thereof” includes any subject that has a disease or disorder associated with a pathological immune response (psoriasis (e.g., mild to moderate psoriasis) or atopic dermatitis (e.g., mild to moderate atopic dermatitis)), as well as any subject with an increased likelihood of acquiring a such a disease or disorder.

The compositions described herein can be used, for example, as a pharmaceutical composition for preventing or treating (reducing, partially or completely, the adverse effects of) an autoimmune disease, such as chronic inflammatory bowel disease, systemic lupus erythematosus, psoriasis, muckle-wells syndrome, rheumatoid arthritis, multiple sclerosis, or Hashimoto's disease; an allergic disease, such as a food allergy, pollenosis, or asthma; an infectious disease, such as an infection with Clostridium difficile; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease, such as chronic obstructive pulmonary disease); a pharmaceutical composition for suppressing rejection in organ transplantation or other situations in which tissue rejection might occur; a supplement, food, or beverage for improving immune functions; or a reagent for suppressing the proliferation or function of immune cells.

In some embodiments, the methods provided herein are useful for the treatment of inflammation. In certain embodiments, the inflammation of any tissue and organs of the body, including musculoskeletal inflammation, vascular inflammation, neural inflammation, digestive system inflammation, ocular inflammation, inflammation of the reproductive system, and other inflammation, as discussed below.

Immune disorders of the musculoskeletal system include, but are not limited, to those conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular immune disorders refers to a immune disorder that affects any structure of the eye, including the eye lids. Examples of ocular immune disorders which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis.

Examples of nervous system immune disorders which may be treated with the methods and compositions described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

Examples of digestive system immune disorders which may be treated with the methods and compositions described herein include, but are not limited to, cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

Examples of reproductive system immune disorders which may be treated with the methods and compositions described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

The methods and compositions described herein may be used to treat autoimmune conditions having an inflammatory component. Such conditions include, but are not limited to, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, good pasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

The methods and compositions described herein may be used to treat T-cell mediated hypersensitivity diseases having an inflammatory component. Such conditions include, but are not limited to, contact hypersensitivity, contact dermatitis (including that due to poison ivy), uticaria, skin allergies, respiratory allergies (hay fever, allergic rhinitis, house dustmite allergy) and gluten-sensitive enteropathy (Celiac disease).

Other immune disorders which may be treated with the methods and compositions include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, sewrum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensistivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autroimmine) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosis, psoriasis, chronic obstructive pulmonary disease, and inflammation accompanying infectious conditions (e.g., sepsis).

### Metabolic Disorders

In some embodiments, the methods and pharmaceutical compositions described herein relate to the treatment or prevention of a metabolic disease or disorder a, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglylceridemia, ketoacidosis, hypoglycemia, thrombotic disorders, dyslipidemia, non-alcoholic fatty liver disease (NAFLD), Nonalcoholic Steatohepatitis (NASH) or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema. In some embodiments, the methods and pharmaceutical compositions described herein relate to the treatment of Nonalcoholic Fatty Liver Disease (NAFLD) and Nonalcoholic Steatohepatitis (NASH).

The methods described herein can be used to treat any subject in need thereof. As used herein, a “subject in need thereof” includes any subject that has a metabolic disease or disorder, as well as any subject with an increased likelihood of acquiring a such a disease or disorder.

The pharmaceutical compositions described herein can be used, for example, for preventing or treating (reducing, partially or completely, the adverse effects of) a metabolic disease, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglylceridemia, ketoacidosis, hypoglycemia, thrombotic disorders, dyslipidemia, non-alcoholic fatty liver disease (NAFLD), Nonalcoholic Steatohepatitis (NASH), or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema.

### Cancer

In some embodiments, the methods and pharmaceutical compositions described herein relate to the treatment of cancer. In some embodiments, any cancer can be treated using the methods described herein. Examples of cancers that may treated by methods and pharmaceutical compositions described herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the methods and pharmaceutical compositions provided herein relate to the treatment of a leukemia. The term “leukemia” includes broadly progressive, malignant diseases of the hematopoietic organs/systems and is generally characterized by a distorted proliferation and development of leukocytes and their precursors in the blood and bone marrow. Non-limiting examples of leukemia diseases include, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemia.

In some embodiments, the methods and pharmaceutical compositions provided herein relate to the treatment of a carcinoma. The term “carcinoma” refers to a malignant growth made up of epithelial cells tending to infiltrate the surrounding tissues, and/or resist physiological and non-physiological cell death signals and gives rise to metastases. Non-limiting exemplary types of carcinomas include, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti.

In some embodiments, the methods and pharmaceutical compositions provided herein relate to the treatment of a sarcoma. The term “sarcoma” generally refers to a tumor which is made up of a substance like the embryonic connective tissue and is generally composed of closely packed cells embedded in a fibrillar, heterogeneous, or homogeneous substance. Sarcomas include, but are not limited to, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Additional exemplary neoplasias that can be treated using the methods and pharmaceutical compositions described herein include Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, plasmacytoma, colorectal cancer, rectal cancer, and adrenal cortical cancer.

In some embodiments, the cancer treated is a melanoma. The term “melanoma” is taken to mean a tumor arising from the melanocytic system of the skin and other organs. Non-limiting examples of melanomas are Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma.

Particular categories of tumors that can be treated using methods and pharmaceutical compositions described herein include lymphoproliferative disorders, breast cancer, ovarian cancer, prostate cancer, cervical cancer, endometrial cancer, bone cancer, liver cancer, stomach cancer, colon cancer, pancreatic cancer, cancer of the thyroid, head and neck cancer, cancer of the central nervous system, cancer of the peripheral nervous system, skin cancer, kidney cancer, as well as metastases of all the above. Particular types of tumors include hepatocellular carcinoma, hepatoma, hepatoblastoma, rhabdomyosarcoma, esophageal carcinoma, thyroid carcinoma, ganglioblastoma, fibrosarcoma, myxosarcoma, liposarcoma, chondrosarcoma, osteogenic sarcoma, chordoma, angiosarcoma, endotheliosarcoma, Ewing's tumor, leimyosarcoma, rhabdotheliosarcoma, invasive ductal carcinoma, papillary adenocarcinoma, melanoma, pulmonary squamous cell carcinoma, basal cell carcinoma, adenocarcinoma (well differentiated, moderately differentiated, poorly differentiated or undifferentiated), bronchioloalveolar carcinoma, renal cell carcinoma, hypernephroma, hypernephroid adenocarcinoma, bile duct carcinoma, choriocarcinoma, seminoma, embryonal carcinoma, Wilms' tumor, testicular tumor, lung carcinoma including small cell, non-small and large cell lung carcinoma, bladder carcinoma, glioma, astrocyoma, medulloblastoma, craniopharyngioma, ependymoma, pinealoma, retinoblastoma, neuroblastoma, colon carcinoma, rectal carcinoma, hematopoietic malignancies including all types of leukemia and lymphoma including: acute myelogenous leukemia, acute myelocytic leukemia, acute lymphocytic leukemia, chronic myelogenous leukemia, chronic lymphocytic leukemia, mast cell leukemia, multiple myeloma, myeloid lymphoma, Hodgkin's lymphoma, non-Hodgkin's lymphoma, plasmacytoma, colorectal cancer, and rectal cancer.

Cancers treated in certain embodiments also include precancerous lesions, e.g., actinic keratosis (solar keratosis), moles (dysplastic nevi), acitinic chelitis (farmer's lip), cutaneous horns, Barrett's esophagus, atrophic gastritis, dyskeratosis congenita, sideropenic dysphagia, lichen planus, oral submucous fibrosis, actinic (solar) elastosis and cervical dysplasia.

Cancers treated in some embodiments include non-cancerous or benign tumors, e.g., of endodermal, ectodermal or mesenchymal origin, including, but not limited to cholangioma, colonic polyp, adenoma, papilloma, cystadenoma, liver cell adenoma, hydatidiform mole, renal tubular adenoma, squamous cell papilloma, gastric polyp, hemangioma, osteoma, chondroma, lipoma, fibroma, lymphangioma, leiomyoma, rhabdomyoma, astrocytoma, nevus, meningioma, and ganglioneuroma.

### Other Diseases and Disorders

In some embodiments, the methods and pharmaceutical compositions described herein relate to the treatment of liver diseases. Such diseases include, but are not limited to, Alagille Syndrome, Alcohol-Related Liver Disease, Alpha-1 Antitrypsin Deficiency, Autoimmune Hepatitis, Benign Liver Tumors, Biliary Atresia, Cirrhosis, Galactosemia, Gilbert Syndrome, Hemochromatosis, Hepatitis A, Hepatitis B, Hepatitis C, Hepatic Encephalopathy, Intrahepatic Cholestasis of Pregnancy (ICP), Lysosomal Acid Lipase Deficiency (LAL-D), Liver Cysts, Liver Cancer, Newborn Jaundice, Primary Biliary Cholangitis (PBC), Primary Sclerosing Cholangitis (PSC), Reye Syndrome, Type I Glycogen Storage Disease, and Wilson Disease.

The methods and pharmaceutical compositions described herein may be used to treat neurodegenerative and neurological diseases. In certain embodiments, the neurodegenerative and/or neurological disease is Parkinson's disease, Alzheimer's disease, prion disease, Huntington's disease, motor neuron diseases (MND), spinocerebellar ataxia, spinal muscular atrophy, dystonia, idiopathicintracranial hypertension, epilepsy, nervous system disease, central nervous system disease, movement disorders, multiple sclerosis, encephalopathy, peripheral neuropathy or post-operative cognitive dysfunction.

### Dysbiosis

The gut microbiome (also called the “gut microbiota”) can have a significant impact on an individual's health through microbial activity and influence (local and/or distal) on immune and other cells of the host (Walker, W. A., Dysbiosis. The Microbiota in Gastrointestinal Pathophysiology. Chapter 25. 2017; Weiss and Thierry, Mechanisms and consequences of intestinal dysbiosis. Cellular and Molecular Life Sciences. (2017) 74(16):2959-2977. Zurich Open Repository and Archive, doi: https://doi.org/10.1007/s00018-017-2509-x)).

A healthy host-gut microbiome homeostasis is sometimes referred to as a “eubiosis” or “normobiosis,” whereas a detrimental change in the host microbiome composition and/or its diversity can lead to an unhealthy imbalance in the microbiome, or a “dysbiosis” (Hooks and O'Malley. Dysbiosis and its discontents. American Society for Microbiology. October 2017. Vol. 8. Issue 5. mBio 8:e01492-17. https://doi.org/10.1128/mBio.01492-17). Dysbiosis, and associated local or distal host inflammatory or immune effects, may occur where microbiome homeostasis is lost or diminished, resulting in: increased susceptibility to pathogens; altered host bacterial metabolic activity; induction of host proinflammatory activity and/or reduction of host anti-inflammatory activity. Such effects are mediated in part by interactions between host immune cells (e.g., T cells, dendritic cells, mast cells, NK cells, intestinal epithelial lymphocytes (IEC), macrophages and phagocytes) and cytokines, and other substances released by such cells and other host cells.

A dysbiosis may occur within the gastrointestinal tract (a “gastrointestinal dysbiosis” or “gut dysbiosis”) or may occur outside the lumen of the gastrointestinal tract (a “distal dysbiosis”). Gastrointestinal dysbiosis is often associated with a reduction in integrity of the intestinal epithelial barrier, reduced tight junction integrity and increased intestinal permeability. Citi, S. Intestinal Barriers protect against disease, Science 359:1098-99 (2018); Srinivasan et al., TEER measurement techniques for in vitro barrier model systems. J. Lab. Autom. 20:107-126 (2015). A gastrointestinal dysbiosis can have physiological and immune effects within and outside the gastrointestinal tract.

The presence of a dysbiosis can be associated with a wide variety of diseases and conditions including: infection, cancer, autoimmune disorders (e.g., systemic lupus erythematosus (SLE)) or inflammatory disorders (e.g., functional gastrointestinal disorders such as inflammatory bowel disease (IBD), ulcerative colitis, and Crohn's disease), neuroinflammatory diseases (e.g., multiple sclerosis), transplant disorders (e.g., graft-versus-host disease), fatty liver disease, type I diabetes, rheumatoid arthritis, Sjögren's syndrome, celiac disease, cystic fibrosis, chronic obstructive pulmonary disorder (COPD), and other diseases and conditions associated with immune dysfunction. Lynch et al., The Human Microbiome in Health and Disease, N. Engl. J. Med 0.375:2369-79 (2016), Carding et al., Dysbiosis of the gut microbiota in disease. Microb. Ecol. Health Dis. (2015); 26: 10: 3402/mehd.v26.2619; Levy et al, Dysbiosis and the Immune System, Nature Reviews Immunology 17:219 (April 2017)

In certain embodiments, exemplary pharmaceutical compositions disclosed herein can treat a dysbiosis and its effects by modifying the immune activity present at the site of dysbiosis. As described herein, such compositions can modify a dysbiosis via effects on host immune cells, resulting in, e.g., an increase in secretion of anti-inflammatory cytokines and/or a decrease in secretion of pro-inflammatory cytokines, reducing inflammation in the subject recipient or via changes in metabolite production.

Exemplary pharmaceutical compositions disclosed herein that are useful for treatment of disorders associated with a dysbiosis contain one or more types of mEVs (microbial extracellular vesicles) derived from immunomodulatory bacteria (e.g., anti-inflammatory bacteria). Such compositions are capable of affecting the recipient host's immune function, in the gastrointestinal tract, and/or a systemic effect at distal sites outside the subject's gastrointestinal tract.

Exemplary pharmaceutical compositions disclosed herein that are useful for treatment of disorders associated with a dysbiosis contain a population of immunomodulatory bacteria of a single bacterial species (e.g., a single strain) (e.g., anti-inflammatory bacteria) and/or a population of mEVs derived from immunomodulatory bacteria of a single bacterial species (e.g., a single strain) (e.g., anti-inflammatory bacteria). Such compositions are capable of affecting the recipient host's immune function, in the gastrointestinal tract, and/or a systemic effect at distal sites outside the subject's gastrointestinal tract.

In one embodiment, pharmaceutical compositions containing an isolated population of mEVs derived from immunomodulatory bacteria (e.g., anti-inflammatory bacterial cells) are administered (e.g., orally) to a mammalian recipient in an amount effective to treat a dysbiosis and one or more of its effects in the recipient. The dysbiosis may be a gastrointestinal tract dysbiosis or a distal dysbiosis.

In another embodiment, pharmaceutical compositions of the instant invention can treat a gastrointestinal dysbiosis and one or more of its effects on host immune cells, resulting in an increase in secretion of anti-inflammatory cytokines and/or a decrease in secretion of pro-inflammatory cytokines, reducing inflammation in the subject recipient.

In another embodiment, the pharmaceutical compositions can treat a gastrointestinal dysbiosis and one or more of its effects by modulating the recipient immune response via cellular and cytokine modulation to reduce gut permeability by increasing the integrity of the intestinal epithelial barrier.

In another embodiment, the pharmaceutical compositions can treat a distal dysbiosis and one or more of its effects by modulating the recipient immune response at the site of dysbiosis via modulation of host immune cells.

Other exemplary pharmaceutical compositions are useful for treatment of disorders associated with a dysbiosis, which compositions contain one or more types of bacteria or mEVs capable of altering the relative proportions of host immune cell subpopulations, e.g., subpopulations of T cells, immune lymphoid cells, dendritic cells, NK cells and other immune cells, or the function thereof, in the recipient.

Other exemplary pharmaceutical compositions are useful for treatment of disorders associated with a dysbiosis, which compositions contain a population of mEVs of a single immunomodulatory bacterial (e.g., anti-inflammatory bacterial cells) species (e.g., a single strain) capable of altering the relative proportions of immune cell subpopulations, e.g., T cell subpopulations, immune lymphoid cells, NK cells and other immune cells, or the function thereof, in the recipient subject.

In one embodiment, the invention provides methods of treating a gastrointestinal dysbiosis and one or more of its effects by orally administering to a subject in need thereof a pharmaceutical composition which alters the microbiome population existing at the site of the dysbiosis. The pharmaceutical composition can contain one or more types of mEVs from immunomodulatory bacteria or a population of mEVs of a single immunomodulatory bacterial species (e.g., anti-inflammatory bacterial cells) (e.g., a single strain).

In one embodiment, the invention provides methods of treating a distal dysbiosis and one or more of its effects by orally administering to a subject in need thereof a pharmaceutical composition which alters the subject's immune response outside the gastrointestinal tract. The pharmaceutical composition can contain one or more types of mEVs from immunomodulatory bacteria (e.g., anti-inflammatory bacterial cells) or a population of mEVs of a single immunomodulatory bacterial (e.g., anti-inflammatory bacterial cells) species (e.g., a single strain).

In exemplary embodiments, pharmaceutical compositions useful for treatment of disorders associated with a dysbiosis stimulate secretion of one or more anti-inflammatory cytokines by host immune cells. Anti-inflammatory cytokines include, but are not limited to, IL-10, IL-13, IL-9, IL-4, IL-5, TGFβ, and combinations thereof. In other exemplary embodiments, pharmaceutical compositions useful for treatment of disorders associated with a dysbiosis that decrease (e.g., inhibit) secretion of one or more pro-inflammatory cytokines by host immune cells. Pro-inflammatory cytokines include, but are not limited to, IFNγ, IL-12p70, IL-1α, IL-6, IL-8, MCP1, MIP1α, MIP1β, TNFα, and combinations thereof. Other exemplary cytokines are known in the art and are described herein.

In another aspect, the invention provides a method of treating or preventing a disorder associated with a dysbiosis in a subject in need thereof, comprising administering (e.g., orally administering) to the subject a therapeutic composition in the form of a probiotic or medical food comprising bacteria or mEVs in an amount sufficient to alter the microbiome at a site of the dysbiosis, such that the disorder associated with the dysbiosis is treated.

In another embodiment, a therapeutic composition of the instant invention in the form of a probiotic or medical food may be used to prevent or delay the onset of a dysbiosis in a subject at risk for developing a dysbiosis.

### Infectious Diseases

The methods of the invention are useful for treating an infectious disease. Infectious diseases are caused by pathogenic microorganisms, such as bacteria, viruses, parasites or fungi; the diseases can be spread, directly or indirectly, from one person to another. Zoonotic diseases are infectious diseases of animals that can cause disease when transmitted to humans.

The methods of the invention are useful for treating viral infection. Viruses are small infectious agents that contain a nucleic acid core and a protein coat, but are not independently living organisms. A virus cannot multiply in the absence of a living cell within which it can replicate. Viruses enter living cells either by transfer across a membrane or direct injection and multiply, causing disease. The multiplied virus can then be released and infect additional cells. Some viruses are DNA-containing viruses and others are RNA-containing viruses. The genomic size, composition, and organization of viruses shows tremendous diversity.

In some embodiments, the viral infection may be caused by an arbovirus, adenovirus, alphavirus, arenaviruses, astrovirus, BK virus, bunyaviruses, calicivirus, cercopithecine herpes virus 1, Colorado tick fever virus, coronavirus, Coxsackie virus, Crimean-Congo hemorrhagic fever virus, cytomegalovirus, Dengue virus, ebola virus, echinovirus, echovirus, enterovirus, Epstein-Barr virus, flavivirus, foot-and-mouth disease virus, hantavirus, hepatitis A, hepatitis B, hepatitis C, herpes simplex virus I, herpes simplex virus II, human herpes virus, human immunodeficiency virus type I (HIV-I), human immunodeficiency virus type II (HIV-II), human papillomavirus, human T-cell leukemia virus type I, human T-cell leukemia virus type II, influenza, Japanese encephalitis, JC virus, Junin virus, lentivirus, Machupo virus, Marburg virus, measles virus, mumps virus, naples virus, norovirus, Norwalk virus, orbiviruses, orthomyxovirus, papillomavirus, papovavirus, parainfluenza virus, paramyxovirus, parvovirus, picornaviridae, poliovirus, polyomavirus, poxvirus, rabies virus, reovirus, respiratory syncytial virus, rhinovirus, rotavirus, rubella virus, sapovirus, smallpox, togaviruses, Toscana virus, varicella zoster virus, West Nile virus, or Yellow Fever virus.

In some embodiments, the viral infection is caused by an enveloped virus. An enveloped virus is an animal virus which possesses a membrane or “envelope,” which is a lipid bilayer containing viral proteins. The envelope proteins of a virus play a pivotal role in its lifecycle. They participate in the assembly of the infectious particle and also play a crucial role in virus entry by binding to a receptor present on the host cell and inducing fusion between the viral envelope and a membrane of the host cell. Enveloped viruses can be either spherical or filamentous (rod-shaped) and include but are not limited to filoviruses, such as Ebola virus or Marburg virus, Arboroviruses such as Togaviruses, flaviviruses (such as hepatitis-C virus), bunyaviruses, and Arenaviruses, Orthomyxoviridae, Paramyxoviridae, poxvirus, herpesvirus, hepadnavirus, Rhabdovirus, Bornavirus, and Arterivirus.

In some embodiments, the viral infection may be caused by influenza A virus, influenza B virus, and influenza C virus. Influenza type A viruses are divided into subtypes based on two proteins on the surface of the virus. These proteins are called hemagglutinin (HA) and neuraminidase (NA). There are 15 different HA subtypes and 9 different NA subtypes. Subtypes of influenza A virus are named according to their HA and NA surface proteins, and many different combinations of HA and NA proteins are possible. For example, an “H7N2 virus” designates an influenza A subtype that has an HA 7 protein and an NA 2 protein. Similarly, an “H5N1” virus has an HA 5 protein and an NA 1 protein. Only some influenza A subtypes (i.e., H1N1, H2N2, and H3N2) are currently in general circulation among humans. Other subtypes such as H5 N1 are found most commonly in other animal species and in a small number of humans, where it is highly pathogenic. For example, H7N7 and H3N8 viruses cause illness in horses. Humans can be infected with influenza types A, B, and C. However, the only subtypes of influenza A virus that normally infect people are influenza A subtypes H1N1, H2N2, and H3N2 and recently, H5N1. The influenza A and B viruses that routinely spread in people (human influenza viruses) are responsible for seasonal flu epidemics each year. It has recently been reported that there is an association between seasonal flu and venous thromboembolism (VTE).

In some embodiments, the viral infection is caused by an arbovirus. Arboviruses are a group of more than 400 enveloped RNA viruses that are transmitted primarily (but not exclusively) by arthropod vectors (mosquitoes, sand-flies, fleas, ticks, lice, etc.). Arborviruses have been categorized into four virus families, including the togaviruses, flaviviruses, arenaviruses, and bunyaviruses. Togaviruses includes the genuses Alphavirus (e.g., Sindbis virus, which is characterized by sudden onset of fever, rash, arthralgia or arthritis, lassitude, headache and myalgia) and Rubivirus (e.g., Rubella virus, which causes Rubella in vertebrates). The Flavivirus genus includes yellow fever virus, dengue fever virus, Japanese encaphilitis (JE) virus, and West Nile virus.

Dengue virus is the most common cause of mosquito-borne viral diseases in tropical and subtropical regions around the world, and is expanding in geographic range and also in disease severity. Currently, there are no licensed drugs for the treatment of dengue. The virus is a small, enveloped, icosahedral virus, with positive strand RNA of 11,000 nucleotides. There are four distinct serotypes of dengue that cause similar disease symptoms, serotypes 1-4 (DENV-1, DENV-2, DENV-3, and DENV-4) that co-circulate in many areas of the world and give rise to sequential epidemic outbreaks when the number of susceptible individuals in the local population reaches a critical threshold and weather conditions favor reproduction of the mosquito vectors Aedes aegypti and Aedes albopictus. Dengue virus infection causes a characteristic pathology in humans involving dysregulation of the vascular system. In some patients with dengue hemorrhagic fever (DHF), vascular pathology can become severe, resulting in extensive microvascular permeability and plasma leakage into tissues and organs. Recently, the mast cell-derived proteases, tryptase and chymase, have been implicated in the immune mechanism by which dengue induces vascular pathology and shock.

West Nile virus is one of the most widely distributed flaviviruses in the world and has emerged in recent years to become a serious public health threat. West Nile virus is an enveloped positive-strand RNA virus, with a genome that encodes 3 structural and 7 non-structural proteins as a single polypeptide that then co- and post translationally processed to yield the 10 proteins. The 3 virus structural proteins are the capsid (C) protein, pre-membrane protein (prM) which is cleaved during virus maturation to yield the membrane (M) protein and envelope (E) protein. The E protein contains the receptor binding and fusion functions of the virus. Severe viral infection is characterized by fever, convulsions, muscle weakness, vision loss, numbness, paralysis, and coma. Because West Nile virus is capable of eliciting pathology in the brain, it has been postulated that the virus may modulate blood-barrier vascular permeability.

In some embodiments, the viral infection is caused by a respiratory syncytial virus (RSV). The respiratory syncytial virus (RSV) is an enveloped, negative-sense, single-stranded RNA virus of the genus Pneumovirinae and of the family Paramyxoviridae. Symptoms in adults typically resemble a sinus infection or the common cold, although the infection may be asymptomatic. In older adults (e.g., >60 years), RSV infection may progress to bronchiolitis or pneumonia. Symptoms in children are often more severe, including bronchiolitis and pneumonia. The RNA genome of the RSV virus is approximately 15 kb and encodes 11 viral proteins, which includes the F (fusion) protein that is a transmembrane protein of the virus and the M (matrix) protein that is a core protein of the virus. RSV infections are known to cause vascular complications and the infection has been associated with venous thromboembolism.

In some embodiments, the viral infection is caused by a coronavirus. Coronaviruses are a family of enveloped, positive-sense, single-stranded RNA viruses, that was first described in 1949. These viruses are found in mice, rats, dogs, cats, turkeys, horses, pigs, and cattle. Occasionally, these viruses infect humans, and the pathology of these viruses in humans is normally not more serious than the common cold.

The coronavirus genome, approximately 27-32 Kb in length, is the largest found in any of the RNA viruses. Large Spike (S) glycoproteins protrude from the virus particle giving coronaviruses a distinctive corona-like appearance when visualized by electron microscopy. The virus is further classified into 4 groups: the α, β, γ, and δ CoVs by phylogenetic clustering, of which α and β are known to cause infection in humans. It is believed that the gammacoronavirus and deltacoronavirus genera may infect humans. Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronaviruses is the Swine Delta Coronavirus (SDCV). Non-limiting examples of betacoronaviruses include Middle East respiratory syndrome coronavirus (MERS-CoV), Severe Acute Respiratory Syndrome coronavirus (SARS-CoV), Severe Acute Respiratory Syndrome coronavirus 2 (SARS-CoV-2), Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV).

Coronaviruses facilitate entry to the cell via protease activation of protein kinases. These kinases are shown to increase the infectivity of the virus by a thousand-fold. Often, virus infection relies on the proteolytic activation of protein kinases (such as elastase in the lung, a serine kinase) as part of the cellular ingress mechanism. Additionally, protein kinases are responsible for signaling pathways regulating inflammation which may be exacerbated by viral triggered, pro-inflammatory cytokine storms which damage organ tissue. Viruses may enter cells through the endosomal pathway. For example, SARS-CoV-2 can use the endosomal pathway, which is reliant on the cysteine proteases cathepsin B and L (CatB/L) and it was shown that blocking these proteases prevented infection. Another protein that is relevant to SARS-CoV-2 pathogenesis is angiotensin-converting enzyme 2 (ACE2), which plays a critical role in coronavirus cellular ingress and expressed in the lung and epithelial cells. ACE2 is a type I transmembrane metallocarboxypeptidase, which has been investigated by several independent researchers as the coronavirus cellular entry receptor and is also responsible for coronavirus attachment.

The first step of coronavirus entry process is the binding of the N-terminal portion of the viral protein unit Si to a pocket of the ACE2 receptor. The second step, which is believed to be of utmost importance for viral entry, is the protein cleavage between the S1 and S2 units by the receptor transmembrane protease serine 2 (TMPRSS2). The cleavage of the viral protein by TMPRSS2 is a crucial step because, after S1 detachment, the remaining viral S2 unit undergoes a conformational rearrangement that drives and completes the fusion between the viral and cellular membrane, with subsequent entry of the virus into cell, release of its content, replication, and infection of other cells. COVID-19 is an infectious disease caused by infection with SARS-CoV-2.

In certain aspects, the methods provided herein comprise treating a viral infection by administering an nanobody disclosed herein. In some embodiments, the viral infection is COVID-19. In some embodiments, the methods further comprise administering to the subject an additional antiviral agent. Antiviral agents are pharmaceutical agents that inhibit viral growth. Such agents may include, but are not limited to, penciclovir, acyclovir, famciclovir, valacyclovir, tenofovir disoproxil fumarate, lamivudine, zidovudine, didanosine, emtricitabine, stavudine, nevirapine, abacavir, raltegravir, dolutegravir, darunavir, ritonavir, cobicistat, efavirenz, ribavirin, neuraminidase inhibitor, recombinant interferons, recombinant immunoglobulins, oseltamivir, zanamivir, peramivir, baloxavir marboxil, remdesivir (RDV), tilorone, favipiravir, IFN-alpha, IFN-beta, 1FN-gamma, IFN-lambda, peginterferon-alpha, peginterferon-beta, ribavirin, lopinavir/ritonavir, TAK888, adefovir, amantadine, rintatolimod (Ampligen), amprenavir, umifenovir (Arbidol), atazanavir, and ivermectin. Therapeutically effective amounts for treatment are familiar to those skilled in the art.

In certain aspects, provided herein are methods comprising inhibiting virus fusion to a human cell with an nanobody disclosed herein. In some embodiments, the virus is SARS-CoV-2. In some embodiments, the virus is hepatitis B virus.

In certain aspects, provided herein are methods for detecting the presence of a virus by contacting the virus with an nanobody disclosed herein, e.g., wherein the nanobody is coupled to a detectable label or other means for determining that the nanobody has bound the virus. In some embodiments, the nanobody binds a viral spike glycoprotein on the virus. In some embodiments, the nanobody binds a human receptor-binding domain of the viral spike glycoprotein. In some embodiments, the virus is SARS-CoV-2. In some embodiments, the virus is hepatitis B virus.

In certain aspects, provided herein are methods for identifying a candidate antiviral agent comprising the steps of a) contacting a viral spike glycoprotein sample with an nanobody disclosed herein, wherein the nanobody comprises a detectable label; b) contacting the viral spike glycoprotein sample with a test agent; c) measuring the binding affinity of the nanobody and test agent to the viral spike glycoprotein; and d) identifying the test agent as a candidate antiviral agent if the binding affinity of the test agent to the viral spike glycoprotein is greater than the binding affinity of the nanobody to the viral spike glycoprotein. In some embodiments, the nanobody binds a human receptor-binding domain of the viral spike glycoprotein. In some embodiments, the virus is SARS-CoV-2. In some embodiments, the virus is hepatitis B virus.

In certain aspects, provided herein are methods for extracting a virus. In these methods, the virus is contacted with an nanobody disclosed herein, wherein the nanobody comprises a detectable label. In some embodiments, the nanobody binds a viral spike glycoprotein on the virus. In some embodiments, the nanobody binds a human receptor-binding domain of the viral spike glycoprotein. In some embodiments, the virus is SARS-CoV-2. In some embodiments, the virus is hepatitis B virus.

In certain aspects, provided herein are test kits for detecting a virus. In some embodiments, the kit comprises a device for collecting a sample. In some embodiments, the kit comprises reagents for detecting the virus, wherein the reagents comprise an nanobody disclosed herein. In certain embodiments, the nanobody comprises a detectable label. In some embodiments, the nanobody binds a viral spike glycoprotein on the virus. In some embodiments, the nanobody binds a human receptor-binding domain of the viral spike glycoprotein. In some embodiments, the virus is SARS-CoV-2. In some embodiments, the virus is hepatitis B virus.

### Examples

Living cellular systems such as bacteria, yeasts, plants, and mammalian cells have become the workhorse for production of a myriad of biologics and compounds. More recently, lyophilized cell extracts (LCE) from living cells have enabled decentralized pharmaceutical production and room temperature storage, which offer opportunities for safe deployment of genetically encoded materials. However, current LCE systems have not been proven to be stable for several years without loss of activity at room temperature, and there is no evidence that these systems are sufficiently resistant to various harsh conditions to allow them to perform their functions in extreme environments.

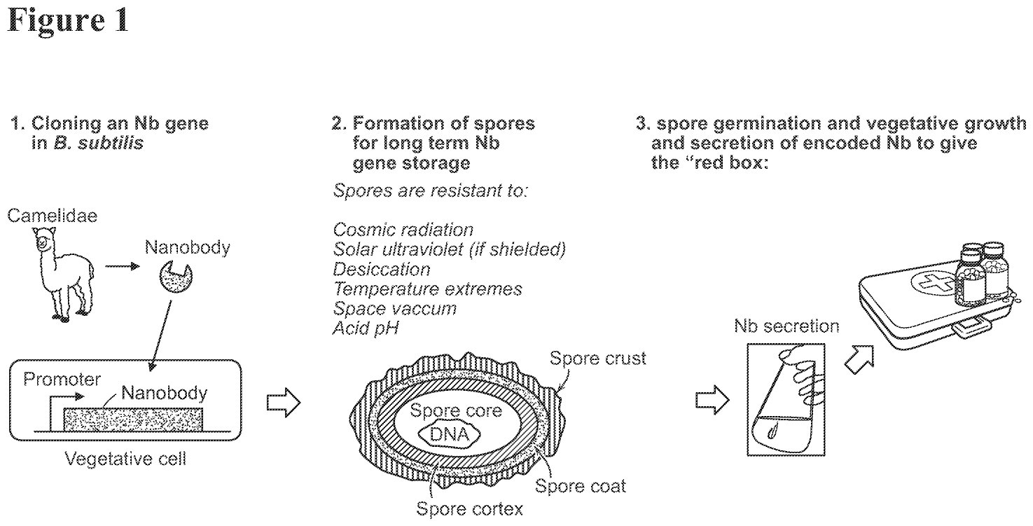

To address the issues noted above, in this work cells of the bacterium Bacillus subtilis were engineered by inserting DNA coding for desired biologics. Bacillus subtilis readily forms highly resistant spores thus allowing long term “gene” storage with excellent stability. Notably, these spores are highly resistant to desiccation and to radiation at doses that exceed the threshold for damage to humans, and spores have been studied for decades to elucidate the biological mechanisms underlying their extreme resistance, as shown in FIG. 1. Despite their dormancy, spores can rapidly return to vegetative growth in one to two hours via germination and outgrowth, and then proliferate in the presence of nutrients. Importantly, in this work, B. subtilis cells were utilized for their vegetative protein expression and secretory systems to design a pipeline to produce nanobodies (Nbs), which are a distinct class of proteins derived from single domain antibody fragments found in camelidae (e.g. camels and llamas) (FIG. 1). These Nbs will be produced with an affinity tag, allowing their purification for eventual antigen detection or purification as well. Nbs have small sizes (12-17 kDa), high solubility, and high thermal stability compared to conventional antibodies. Due to their small sizes, Nbs are particularly suited for single molecule imaging at nanoscale resolution, clinical diagnostics such as Positron Emission Tomography—Computed Tomography imaging, and therapeutics for a variety of diseases. Currently, most, if not all, Nbs are produced intracellularly in microbial cells, predominantly E. coli. However, Nb isolation from E. coli cells requires lysis of the bacteria, which may significantly prolong the downstream processing time. Additionally, E. coli contains endotoxins which can cause multiple undesirable effects in humans, and thus E. coli extracts require complete removal of endotoxins in the development of Nbs produced in this organism as therapeutic biologics.

In addition to in E. coli, production of Nbs has been explored in yeast and Brevibacillus expression systems or through display of Nbs on the surface of B. subtilis spores for sensing, but using B. subtilis to secrete Nbs into the medium has not previously been considered. One simple advantage in using this Gram-positive bacterium is that it contains minimal endotoxin levels, and thus extracellular production of Nbs with extremely low endotoxin levels in B. subtilis could simplify downstream purification. B. subtilis is well known to secrete many proteins it synthesizes. However, many eukaryotic proteins (e.g. mammalian) are poorly produced by the Sec-dependent secretion pathway in B. subtilis, likely because this secretion system requires an unfolded state of target proteins during export of proteins from the cytoplasm to the environment, where abundant proteases are also secreted by B. subtilis. Although protease-deficient B. subtilis strains have been developed to mitigate the problem of proteolysis, secreting some intact mammalian proteins in high yields by B. subtilis remains problematic, possibly due to misfolding of target proteins. In planning this work, however, it was speculated that Nbs high thermostability and relatively small size may render these proteins amenable to the B. subtilis secretory system. Indeed, it was demonstrate that a panel of four different Nbs specific for small molecules (caffeine and methotrexate) or eukaryotic cell surface-associated protein antigens, Cytotoxic T-Lymphocyte Associated Protein 4 (CTLA-4) and Programmed Death-Ligand 1 (PD-L1) can be readily secreted in an intact state and at high yields (up to 20 mg protein per liter in a shake-flask mode) by B. subtilis. In addition, by fusing an anti-caffeine Nb with a cellulose-binding domain (CBD), the Nb can be immobilized on cellulose-based materials, including thin paper and columns, for long-term storage or small molecule capturing on a low-cost substrate. Importantly, direct secretion of Nbs in the B. subtilis culture supernatant fluid can potentially enable the rapid detection of antigen targets without purification. In turn, the latter advances may also pave the way for the development of low-cost, portable and extremely stable detection systems, and even biologics production systems, all in extreme environments (Engineering Bacillus subtilis as a Versatile and Stable Platform for Production of Nanobodies, Yang et al. 2020).

### Example 1: Schematic Pipeline for Expression of Nanobodies (Nbs), their Purification and Use for Antigen Purification

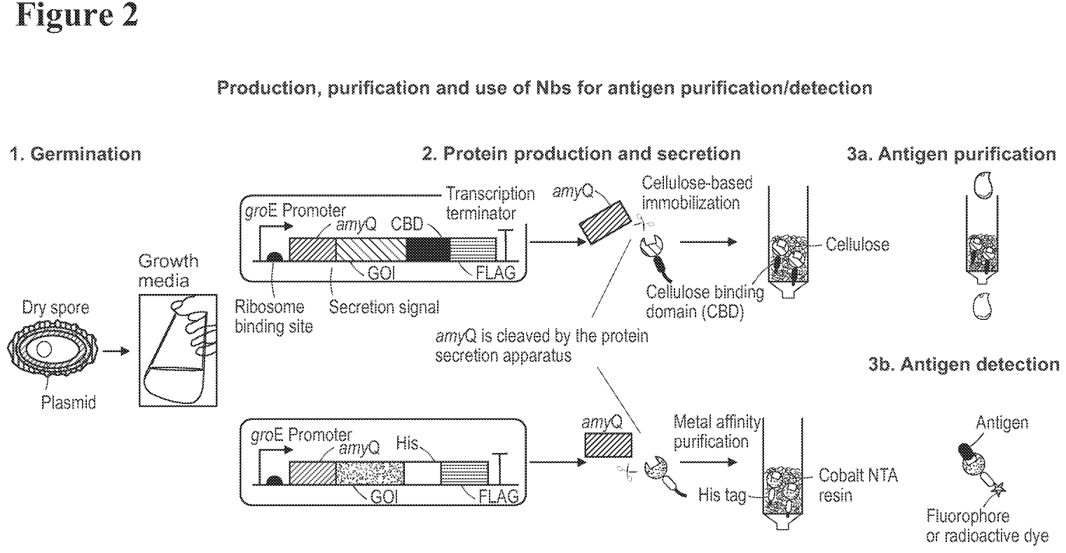

The overall scheme for ultimate purification of antigens is outlined in FIG. 2. The pipeline begins with transformation of B. subtilis with the gene for the Nb selected plus appropriate signals for transcription and translation of the gene in growing cells and excretion of the protein. The secreted Nb will contain either a His or a CBD tag allowing easy purification of the protein on an appropriate affinity column. The column bound protein can then be used to purify the Nb's antigen, or the Nb can be eluted for use in antigen detection.

Vegetative cells of B. subtilis were transformed with plasmids that encode genes of interest (GOI) such as a particular Nb, and the Nb is fused with a cellulose binding domain (CBD) to allow for cellulose-based immobilization or with a 6×Histidine tag (His-tag) to enable metal affinity purification on a cobalt nitriloacetic acid (NTA) resin. The column bound protein can then be used to purify the Nb's antigen (e.g. small molecules), or the Nb can be eluted for use in antigen detection via conjugation of fluorescent dyes or radioisotope labelling. For long-term storage, B. subtilis are induced to form spores, and when the Nb is needed, the spores can be inoculated in culture media to enable spore germination, outgrowth and subsequent vegetative growth, and then synthesis and secretion of the Nb via the Sec-dependent protein secretion pathway.

### Example 2: Extracellular Secretion of Nanobodies (Nbs) in B. subtilis

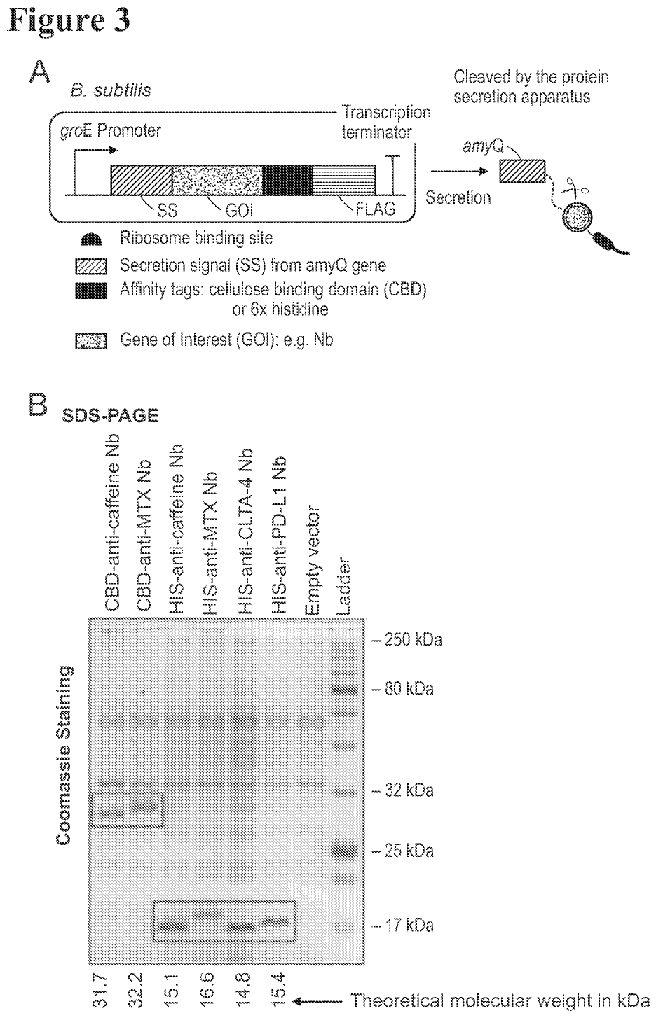

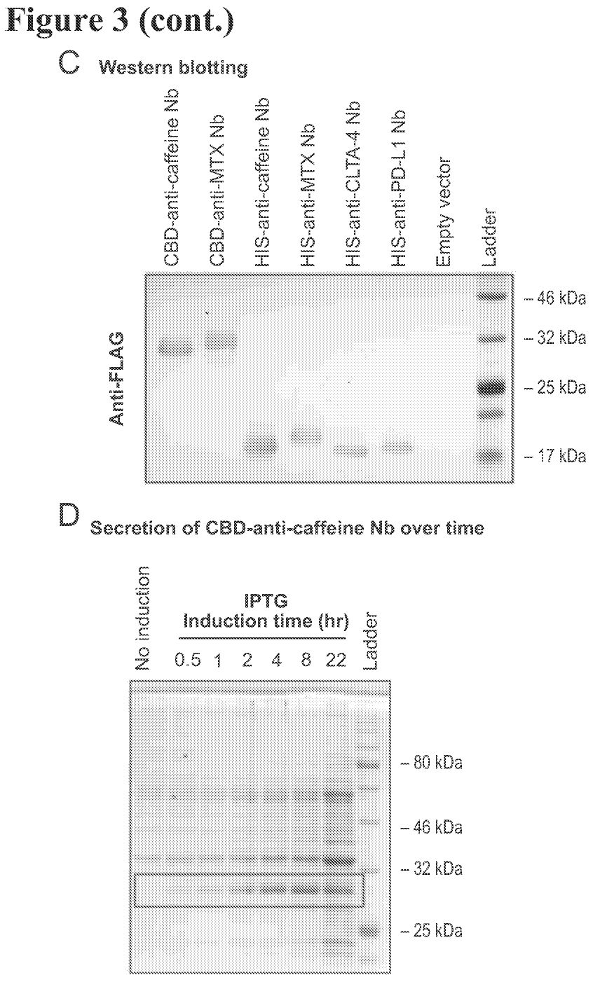

B. subtilis secretes proteases into the culture supernatant, which can dramatically lower the yields and quality of secreted proteins. To minimize this problem, a modified B. subtilis strain, WB800N, was used, which is deficient in eight extracellular proteases. The final fusion gene cassette was cloned into a vector named pHT43, which contains a strong promoter that is derived from the B. subtilis groE operon and has been converted into an isopropyl-β-D-thiogalactoside (IPTG)-inducible promoter by addition of the lac operator from E. coli. Downstream of the promoter region are the ribosome binding site (RBS) and the amyQ secretion signal sequence (FIG. 3, Panel A). Because the amyQ secretion signal sequence requires the bacterial Sec pathway to export proteins extracellularly, exported proteins such as Nbs must first be unfolded to thread through the protein export machinery anchored on the bacterial membrane. As a result, many mammalian recombinant proteins have failed to be secreted at detectable levels due to degradation or misfolding. However, it was hypothesized that recombinant proteins that are highly thermally stable and/or have relatively small molecular weights, such as Nbs, may not be subject to this problem in the Sec-dependent secretion pathway in B. subtilis. To confirm this hypothesis, four different Nbs were produced: (1) anti-caffeine; (2) anti-methotrexate (MTX); (3) anti-mouse CTLA-4 and (4) anti-mouse PD-L1. Primers of the genes of interest used in the study are shown in Table 4. The first two Nbs bind small molecules with potential applications in small molecule purification, while the latter two detect surface antigens on immune cells and tumor cells with potential clinical diagnostic applications. Notably, these four Nbs, two with the CBD tag and two with the His-tag, were readily detected in the supernatant of bacterial cultures after IPTG induction for six hr in comparison to that of empty vector-transformed bacteria (FIG. 3, Panel B). The Nbs yields were estimated to be 15-20 mg from one liter of bacterial culture in a shake-flask mode, which is comparable to yields in shake-flask-based host systems for Nb production in E. coli. To confirm that the protein bands detected on Sodium Dodecyl Sulfate Polyacrylamide Gel Electrophoresis (SDS-PAGE) were truly the proteins of interest, Western blotting was performed using an antibody specific for the FLAG epitope cloned in the fusion proteins. As shown in FIG. 3, Panel C, the fusion proteins' migration positions on SDS-PAGE were those expected based on their calculated molecular weights shown in FIG. 3, Panel B.

Extracellular expression bypasses the need for lysing bacteria to extract proteins but may have relatively slower production rates than those of intracellular expression, due to the requirement for proteins to unfold and refold when using the Sec-dependent secretion pathway. To understand the kinetics of Nb secretion from B. subtilis, bacterial culture supernatants were collected at different times after addition of the inducer IPTG. Following TCA precipitation of total proteins from the supernatant, Nbs with the His tag (PD-L1 and CTLA-4) or the CBD tag (caffeine) were detectable by SDS-PAGE as early as 0.5 hr after induction and levels increased up to 4 hr (FIG. 3, Panel D). These results suggest that when an inducible protein secretion system is desirable in B. subtilis, protein secretion can occur within a 1-hr timeframe, which can enable the on-demand production of Nb-based biologics.

TABLE 4
Primers used for genes of interest in study

| Target | Primer | |
|---|---|---|
| gene | name | Sequence |
| FLAG- | B83 | tacaaaaacatcagccgtaggatcc |
| CBM3-TEV | | CATCATCATCATCATCACAGC |
| | B84 | gtacttgggaTCCCTGGAAGTACAG |
| | | GTTTTC |
| hPTH | B136 | cttgaagtcctctttcagggaccc |
| | | AGCGTTTCGGAAATTCAATTG |
| | B137 | ctgccccggggacgtcgactctaga |
| | | CTATTAGAAATTGTGCACATCC |
| Caffeine/ | B85 | cttccagggaTCCCAAGTACAACTG |
| MTX | | GTAG |
| | B86 | ctgccccggggacgtcgactctaga |
| | | CTATTAGCTGCTTACGGTTAC |
| hG-CSF | B138 | cttgaagtcctctttcagggaccc |
| | | ACGCCGTTGGGCCCCGCC |
| | B139 | ctgccccggggacgtcgactctaga |
| | | CTATTACGGCTGTGCAAGATGACGCAA |
| PD-L1 & | B145 | ACAAAAACATCAGCCGTAGGA |
| CTLA-4 | B146 | CCGGGGACGTCGACTCTAG |

Capital letters are complementary to the gene of interest. Lower letters are sequences that overlap with the plasmid backbone.

### Example 3: The Fusion Protein Consisting of a CBD and a Nanobody (Nb) is Functionally Active Following Secretion from B. subtilis

Immobilizing thermostable Nbs on a substrate such as cellulose with low cost and a long shelf life may have implications for the detection of small molecules using hapten-specific Nbs under extreme conditions. To this end, the investigation focused on the CBD from the cipA gene in the bacterium C. thermocellum. This CBD has been shown to be resistant to denaturation at high temperatures (Tm=70° C.) due to the thermophilic nature of C. thermocellum. While this CBD has been used as an affinity tag to enable protein immobilization and purification, it has been predominantly expressed inside E. coli to make recombinant fusion proteins, which necessitates lysis of bacteria via enzymes (e.g. lysozyme). In contrast, the ease of producing recombinant proteins extracellularly by the Sec-dependent secretory pathway in B. subtilis was motivating to produce fusion proteins consisting of a CBD and an Nb. It was speculated that the small size and high thermostability of the CBD (˜17 kDa) may allow for fast refolding and resistance to protease-mediated degradation, which has been a problem for heterologous protein expression using the Sec-dependent secretion system in B. subtilis.

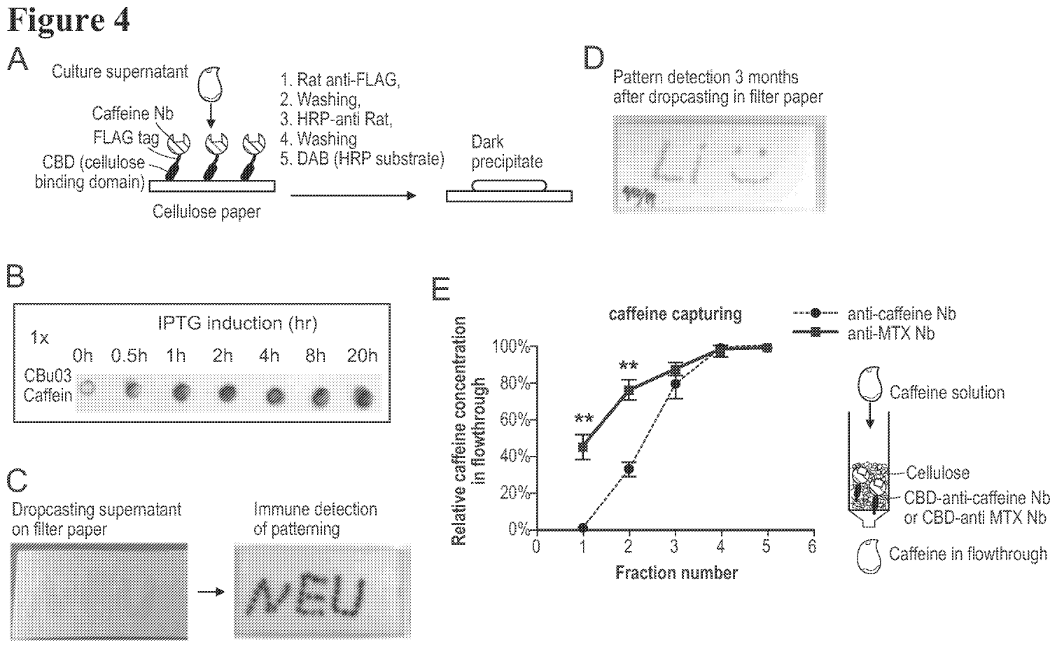

To determine whether CBD fusion proteins were bifunctional (i.e. cellulose binding and Nb-specific target recognition), the investigation focused on CBD-anti-caffeine Nb as a proof of concept. First, supernatant fluid containing secreted fusion proteins was spotted on a piece of cellulose filter paper (FIG. 4B, circled areas); upon air drying, the paper was stained with a rat antibody against the FLAG epitope in the fusion protein, followed by an anti-rat secondary antibody conjugated with horseradish peroxidase (RP) and HRP substrates, yielding a black precipitate (FIG. 4 Panel A). As shown in FIG. 4, Panel B, black staining inside the circles indicated binding of the fusion protein to the cellulose paper, and the protein abundance in the supernatant increased with induction time, as evidenced by the increase in intensity of the black staining. As a negative control, no fusion protein was detected in the supernatant from uninduced (i.e. 0 hr) bacteria. Alternatively, a pattern of “NEU” (Northeastern University) was generated on cellulose paper using the same supernatant and detected the pattern by immunoblotting (FIG. 4, Panel C). It was also demonstrated that the stability of the immobilized fusion proteins three months after application to and drying the fusion protein on filter paper using a different pattern, with the original sample still detectable by immunoblotting (FIG. 4, Panel D). Therefore, B. subtilis was successfully utilized to secrete a CBD-based fusion protein that can be immobilized on cellulose, with potential applications in biosensing or biologic storage based on cellulose paper.

To confirm that the Nb component in the CBD-Nb fusion protein retained its target recognition ability, a chromatography column prototype was devised by packing a 1 ml syringe with 0.1 ml regenerated amorphous cellulose (RAC) (˜50 mg dry weight) (FIG. 4, Panel E). RAC was prepared by hydrolyzing cellulose microcrystals with 85% phosphoric acid, such that the hydrolyzed cellulose has a larger surface area to maximize its binding to the CBD. Next, the column was loaded with bacterial supernatant containing the fusion protein and monitored the saturation of the column by detecting the presence of CBD-anti-caffeine Nb from the flow-through via SDS PAGE. By quantifying the amount of CBD-anti-caffeine Nb in the original supernatant and the flow-through fractions, in which the fusion proteins were found to saturate the column, it was estimated that ˜0.5 mg of CBD-anti-caffeine Nb was bound to 0.1 ml RAC (˜50 mg dry weight). These procedures in principle converted a cellulose-based column to an affinity purification system, where the CBD-Nb fusion protein serves as a bridge between a low-cost, stable immobile phase (i.e. the cellulose substrate) and molecules of interest from the mobile phase (e.g. caffeine). As a proof of concept for purification of Nbs' antigens, 150 μg/ml caffeine solution prepared in phosphate buffered saline (PBS) was applied to a RAC column carrying immobilized CBD-anti-caffeine Nb or CBD-anti-MTX Nb (a negative control), collected five flow-through fractions (100 μl per fraction), and the fractions were analyzed with a UV spectrophotometer. The measurements showed that caffeine levels in the mobile phase were significantly reduced when passing through the RAC column that carried the CBD-anti-caffeine Nb, in comparison to the CBD-anti-MTX Nb, thus demonstrating specific target recognition by the secreted CBD-anti-caffeine Nb (FIG. 4, Panel E), compared to non-specific caffeine adsorption by the RAC column carrying the nonspecific Nb, CBD-anti-MTX Nb. Using data from only the first three flow-through fractions from the RAC column with the CBD-anti-caffeine Nb and subtracting the nonspecific caffeine binding to the column, indicated that there was ˜10 μg caffeine bound to the column that had ˜500 μg immobilized CBD-anti-caffeine Nb fusion protein; this amount of caffeine bound is close to one caffeine per one fusion protein. Consequently, these results demonstrate that the two components in the CBD-anti-caffeine Nb fusion protein preserved their respective functions upon secretion from B. subtilis. Moreover, directly applying the supernatant containing the desired CBD-Nb fusion proteins to a cellulose-based column conveniently generates a simple chromatography system to capture small molecules. This approach may be highly tunable for target binding and purification by swapping the Nb component in the fusion protein.

### Example 4: Detection of Protein Antigens Using (Nanobodies) Nbs Secreted from B. subtilis

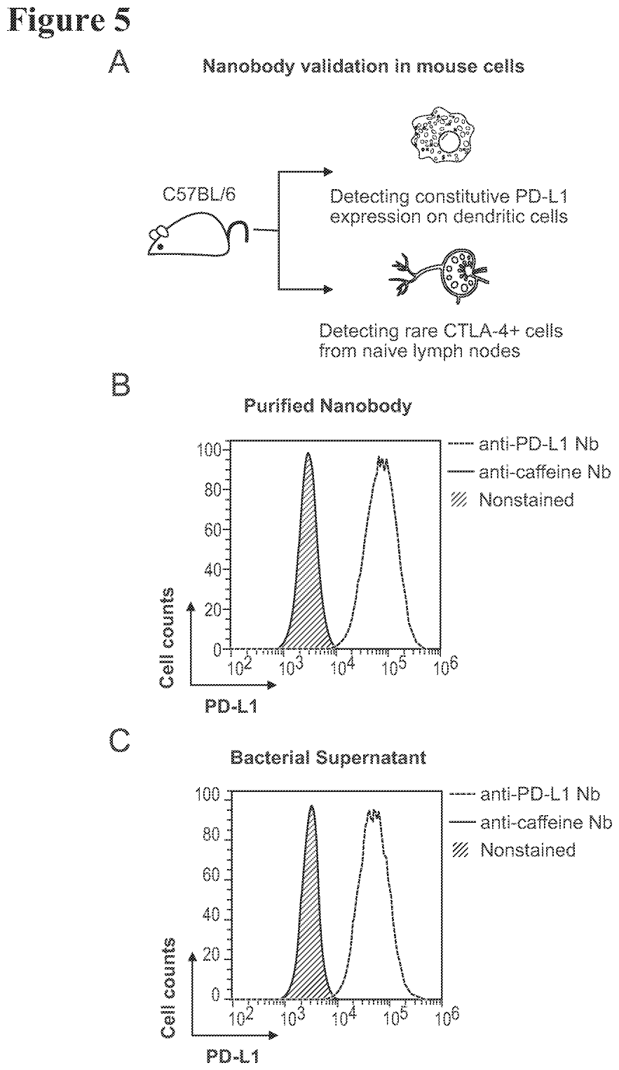

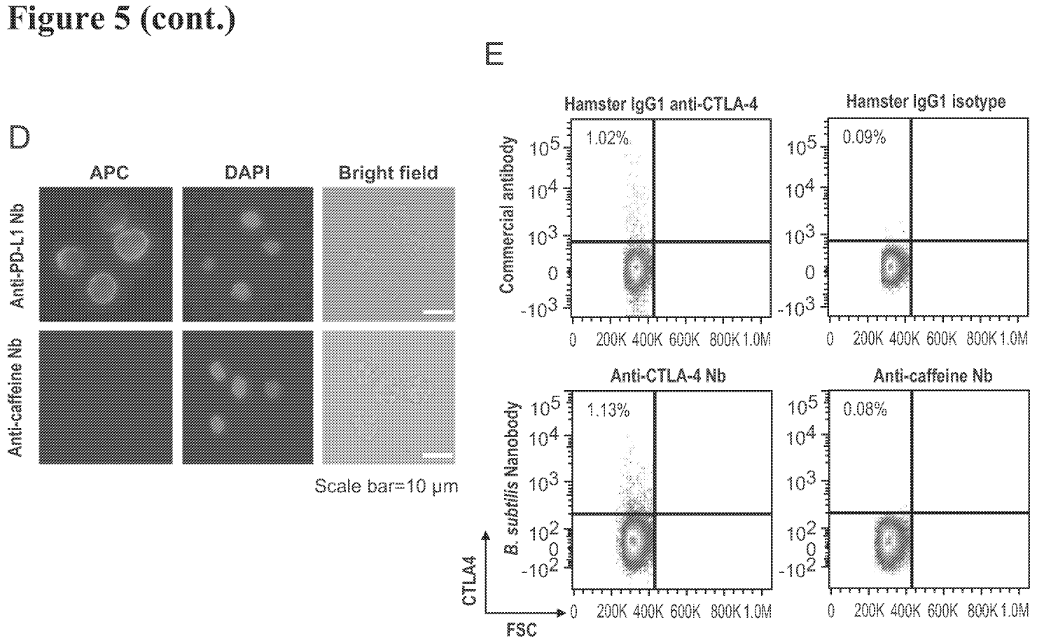

Nanobodies for immune checkpoint blockade (ICB) such as anti-PD-L and CTLA-4 have found utility in cancer immunotherapy as well as in stratifying tumor specimens to inform patients' responses to FDA-approved ICB therapeutics. While the majority of anti-PD-L1 and anti-CTLA-4 Nbs are currently produced intracellularly in E. coli, the lengthy processes of lysing bacteria and removing endotoxins may dramatically increase costs and demand a centralized facility for production. In contrast, bacterial culture supernatant containing His tagged anti-PD-L1 or anti-CTLA-4 Nb were directly loaded to cobalt NTA agarose resin in a column format as illustrated in FIG. 2 Panel B, and the proteins subsequently eluted from these columns exhibited high purity and homogeneity. Of note, in the shake-flask expression system, anti-PD-L1 and anti-CTLA-4 Nbs can be secreted into the supernatant of B. subtilis culture at ˜15 mg per liter of culture. To further confirm that these Nbs remain functional following secretion and purification, the mouse dendritic cell line DC 2.4 was chosen because it is known to constitutively express the immunoinhibitory receptor PD-L1 on the cell surface (FIG. 5, Panel A), and it was also validated the expression of PD-L1 by a commercial antibody specific for mouse PD-L1. DC 2.4 cells were immunostained with purified anti-PD-L1 Nb or anti-caffeine Nb, followed by a fluorophore-conjugated anti-FLAG antibody to detect the FLAG epitope that was fused to the Nbs. The stained samples were analyzed by flow cytometry and fluorescence microscopy—results of flow cytometry indicated the specific binding of purified anti-PD-L1 Nbs and no binding for the anti-caffeine Nb control in comparison to the unstained sample (FIG. 5, Panel B). In agreement with the flow cytometry data, imaging by fluorescence microscopy showed positive staining of PD-L1 on the surface of DC 2.4 cells, and the absence of binding with the anti-caffeine Nb (FIG. 5, Panel C). To further test whether the purification step could be bypassed by directly using bacterial supernatant, 200 μl of the original B. subtilis supernatant containing the anti-PD-L1 Nb was applied to stain ˜one million DC 2.4 cells for fluorescence microscopy. As shown in FIG. 5, Panel C, the bacterial supernatant with the PD-L1 specific Nb detected PD-L1 expression on DC 2.4 cells, but anti-caffeine Nb (negative control) did not.

In addition to detecting abundant antigens on a homogeneous population of cells such as the immortalized dendritic cells DC 2.4, identification of targets present in only a low percentage of cells (e.g. ˜1%) (FIG. 5, Panel D) was performed, which may have implications in the development of sensitive diagnostic reagents using B. subtilis-secreted Nbs. According to the literature, CTLA-4 has a very low level of expression in unstimulated (naïve) T lymphocyte cells, and only a small fraction of cells is positive for CTLA-4 (29, 30). As shown in FIG. 5, Panel E, ˜1% of CTLA-4 positive cells in unstimulated primary cells, which were harvested from lymph nodes in C57BL/6 mice, were detected by both a commercially available antibody specific for mouse CTLA-4 and the anti-CTLA-4 Nb purified from the supernatant of B. subtilis. Consequently, it was conclude that Nbs secreted by B. subtilis are functional, and this new Nb production platform has potential applications in generation of Nb-based diagnostics and drug development.

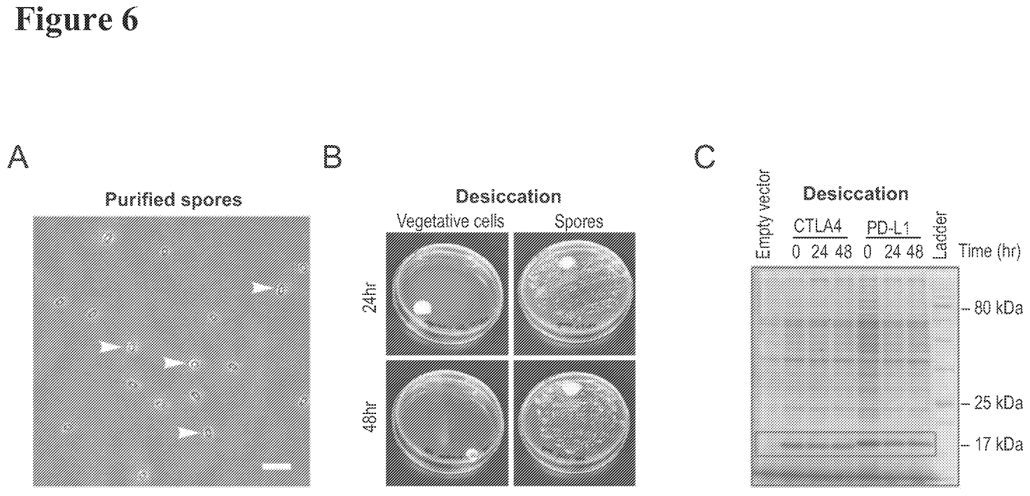

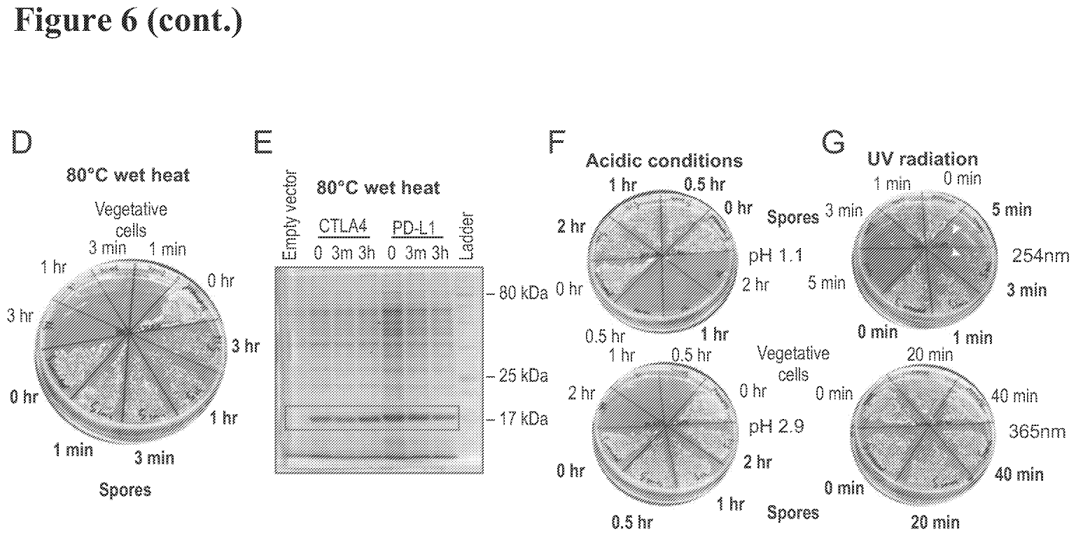

Example 5: Development of a highly resistant B. subtilis spore-based biologic production platform Upon nutrient starvation, B. subtilis can undergo sporulation to generate spores, and these spores have been studied for decades to understand the biological mechanisms underlying their extreme resistance. Despite their dormancy and resistance, spores can rapidly return to vegetative growth within 2 hr in the presence of nutrients. In light of these findings, it was asked: can spores be used to store genetic information that encodes and programs the generation of Nbs under extreme environments? To this end, vegetative cells of B. subtilis carrying genes for Nbs were induced to form spores and then followed established protocols to obtain highly purified spores which are essential for studies on spore resistance, killing, and germination (FIG. 6, Panel A). Afterwards, purified spores were exposed to four environmental extremes that are relevant to different applications: (1) desiccation; (2) wet heat (80° C.); (3) ultraviolet irradiation (UV₂₅₄ₙₘ and UV₃₆₅ₙₘ) and (4) acidic pH.

First, to enable long-term storage and great portability, it is desirable to store the engineered bacteria in a dry state, and when needed they can outgrow and produce Nbs of interest. However, growing B. subtilis cells are vulnerable to desiccation. To demonstrate that spores that carry the Nb-encoding plasmid construct can serve as a stable platform against desiccation, spores and vegetative cells (at an OD₆₀₀ₙₘ of 1.0) were applied to a piece of filter paper followed by air drying, and stored them at 37° C. for 24 and 48 hr. At 24 and 48 hr time points, filter paper carrying spores or vegetative cells was placed on agar growth plates and streaked to allow for recovery and colony formation. The result of the desiccation resistance test shows that spores were able to successfully germinate and proliferate, but the vegetative cells did not (FIG. 6, Panel B). Moreover, when the same desiccation-treated spores were inoculated into growth media, they were able to produce Nbs (anti-CTLA-4 and anti-PD-L1) at a level comparable to that of freshly inoculated spores (FIG. 6, Panel C).

Next, engineered spores and a vegetative cell controls, both of which express the anti-CTLA-4 Nb, were suspended at an OD of 0.01 and incubated at 80° C. for 1 min, 3 min, 1 hr and 3 hr. After each time point, spore and vegetative cell suspension were plated for recovery and colony formation. The result of the heat resistance test shows that spores were able to germinate and proliferate after heat exposure for up to 3 hr, but the vegetative cells did not survive even 1 min exposure (FIG. 6, Panel D). Moreover, when the same heat-treated spores were inoculated into growth media, they were able to produce Nbs (anti-CTLA-4 and anti-PD-L1) at a level comparable to that of the untreated strain (FIG. 6, Panel E).

Thirdly, some B. subtilis strains have been commercialized as probiotics to promote health. Although the exact mechanisms underlying how probiotics mediate health benefits remain elusive and subject to debate, one potential application is to explore B. subtilis as a chassis to deliver Nbs to address diseases in the gastrointestinal tract (GIT). For example, studies have shown that B. subtilis strains can persist in the mouse GIT for up to 27 days. However, with oral delivery, probiotics must first survive the deleterious environment in the stomach, which has an extremely acidic pH. To examine the resistance of spores to acidic pH, engineered B. subtilis spores and vegetative cells encoding the Nb specific for CTLA-4, were incubated at pH 1.1 or 2.9 with 0.9% NaCl to maintain an isotonic condition. The viabilities of the spores and control group were measured at 0.5-2 hr, in the range of estimated durations for spores to transit through the stomach. The results showed that spores survive these acidic conditions and can both germinate and grow vegetatively after nutrients are provided. Conversely, the vegetative cells incubated in acidic pH were unable to grow after nutrients were provided (FIG. 6, Panel F). Importantly, after a 2-hr challenge at acidic pH, the engineered spores remained able to germinate, grow and produce recombinant Nb upon IPTG induction, with expression levels comparable to those of neutral pH-treated spores.

Finally, the resistance of engineered spores to UV light of two different wavelengths (254 nm and 365 nm) was explored by holding a UV lamp with dual wavelength at a distance of 6 cm perpendicular to spores suspended in PBS, and with this setting the intensities of UV radiation are 760 (254 nm) and 720 (365 nm) μW/cm². As expected, under UV at 254 nm, both spores and vegetative cells failed to survive when the exposure time passed 5 min, but spores showed higher viability than vegetative cells (FIG. 6, Panel G). Of note, colonies from spores which were recovered after UV₂₅₄ₙₘ exposure times of 1 min, 3 min and 5 min, remained able to produce the anti-CTLA-4 Nb after inoculation in medium and induction with IPTG. In contrast to UV₂₅₄ₙₘ treatment, both spores and vegetative cells survived exposure to UV light at 365 nm even after irradiation for 40 min, and with no noticeable differences in viability (FIG. 6, Panel G). These results suggest that spores will need to be shielded against short wavelength UV such as 254 nm if engineered spores are to be utilized as a biologics production platform in space exploration.

### CONCLUSIONS

In this work, a new approach was presented for engineering B. subtilis to secrete Nbs directly into culture media. As a proof of concept, it was shown that four different Nbs targeting either small molecules (caffeine and methotrexate) or cell surface-associated protein antigens (PD-L1 and CTLA-4) can be readily produced in and secreted from B. subtilis giving 15-20 mg of Nbs per liter of culture supernatant in the shake-flask mode, which can enable facile purification and streamline many downstream applications. Notably, this yield is comparable to that of reported for E. coli-based systems for extracellular secretion of Nbs, although intracellular expression of Nbs in E. coli has been shown to reach ˜100 mg/L through extensive optimization of growth conditions. However, intracellular expression does not have the convenience of harvesting proteins directly from the supernatant without the need to lyse bacteria, and the latter may be particularly cumbersome for production of Nbs in a large scale. The yield could be improved by using a fed-batch mode to scale up Nbs production and excretion in B. subtilis. Additionally, it was shown that when genetically fusing the caffeine-specific Nb to a cellulose-binding domain, bacterial supernatant containing the fusion protein can be directly applied to a cellulose-based substrate for long term storage or be integrated into an affinity-based chromatography system for caffeine capture. Importantly, this strategy could be adapted to bind and purify other small molecules by swapping the caffeine-specific Nb component in the fusion protein with other hapten-specific Nbs.

The ability to produce Nbs that detect immune checkpoint ligands such as PD-L1 or receptors such as CTLA-4 may also be useful in the field of cancer immunotherapy, and could also be extended to generate Nbs that block inflammatory cytokines such as TNF-α to diagnose or treat inflammation. In this work, the FLAG epitope was cloned at the C terminus of Nbs as a molecular handle for Nb detection via indirect immunostaining. To reduce the indirect staining step, the FLAG epitope can be a replaced with a sortase tag to covalently conjugate Nbs with fluorescent dyes or other peptide sequences via sortase-mediated protein ligation. Additionally, this secretory system may be integrated with other advanced technologies such as non-natural amino acids to allow for side-specific conjugations and labeling. Finally, as expected, the engineered B. subtilis strains can be induced to form spores via nutrient starvation, and these engineered spores can survive desiccation, extreme ambient heat and acidic pH very well, and UV₂₅₄ exposure to certain extent. Importantly, spores surviving these treatments were still able to secrete their encoded Nb after spore germination, outgrowth and growth. These resistance properties may then allow spores to serve as a highly stable storage form for bacteria that might be needed to function in an environment such as deep space. While spores can survive for many years in a desiccated state, upon rehydration and exposure to nutrients, spores rapidly transition back to growing cells, thus providing the capacity to manufacture and purify desired pharmaceuticals.

### Example 6: An Integrated Sampling and Diagnosis of COVID-19 on a Cotton Swab

The first line of prevention of COVID-19 is to quickly identify areas with exposure to COVID-19. For instance, it has been clear that COVID-19 are transmitted through skin contact with solid surfaces and aerosol particles that carry COVID-19. Although disinfection on large suspected areas with bleach and alcohol are proven effective in neutralizing virus in public places (e.g. hospitals, grocery stores and public transportation), it is not practical or economical to apply this type of “blinded” approach to disinfect all surface areas. It is reasoned that there is an immediate need to identify the source of the contamination in those public areas to track and de-risk the exposure to COVID-19 on solid surfaces as well as skins from infected and asymptomatic humans.

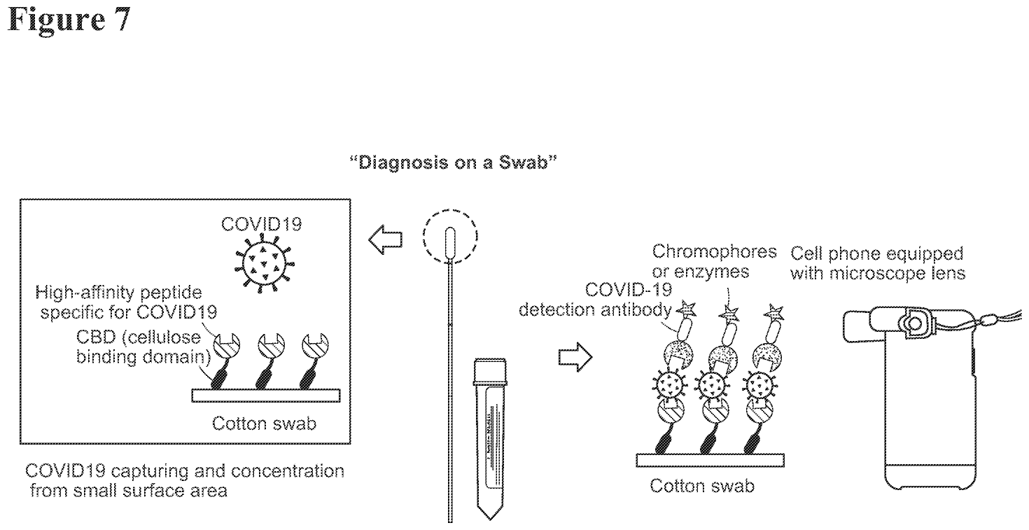

Motivated by hand swabbing for explosive detection at checkpoints and airport gates, the aim of this work was to develop a swabbing method to simultaneously sample and detect COVID-19 on surfaces. However, it is challenging to detect the low densities of COVID-19 present on most surfaces via existing diagnostic tests since those tests are optimized for high viral titers such as those found in saliva and blood from COVID-19-infected patients. Here, it was proposed to develop a functional swab that can capture COVID-19 from a large surface area via COVID-19-specific binding peptides, perform a gold standard immune assay on the swab, and rapidly acquire results facilitated by smart phones with commercially available microscope lens (FIG. 7). In doing so, this “sampling and diagnosis on a swab” technology can become a powerful point-of-care device, based on a cellulose-based immunoblot assay. Moreover, this technology features portability, room temperature storage, dry form, long shelf life, and high specificity.

First, noninfectious recombinant COVID-19 spike proteins that are complexed with RNA fragments as the proxies for intact virus will be produced and will be them on a surface at different densities to mimic contaminated areas. Second, at least three different FDA-approved cotton swabs the contact time and wetness of the swabs to efficiently capture and remove COVID-19 “ghost” from the surface to immobilize the cellulose-bound and COVID-19-specific fusion protein. The contact time and wetness of the swabs will be optimized to efficiently capture and remove COVID-19 from the surface. The final step is the direct immune detection of COVID-19 on the swabs via a smartphone. The spike-protein-based detection will be benchmarked against RNA detection, where RNA will be extracted from the swabs and quantitative PCR with both positive and negative controls will be used to evaluate the sensitivity (FIG. 7).

### Example 7: A Highly Stable and Versatile Diagnostic Platform Against Hepatitis B Virus

Hepatitis B is a severe liver infection that attacks the liver and can cause both acute and chronic diseases by the hepatitis B virus (HBV). As of 2016, 27 million people (10.5% of all people estimated to be living with hepatitis B) were aware of their infection, while 4.5 million (16.7%) of the people diagnosed were on treatment. Laboratory confirmation of the diagnosis is essential, for it is not possible, on clinical grounds, to differentiate hepatitis B from hepatitis caused by other viral agents.

The diagnosis of HBV infection relies heavily on serological and molecular tests. They can 1) directly provide the instructions to the doctors about the medication and diagnose 2) directly reflect the replication status and infectivity of HBV, and 3) directly reflect the activation degree of the virus and indirectly reflect the self-immune response level.

Serological tests use serum-based blood tests to identify virus-encoded antigens and their corresponding antibodies: hepatitis B surface antigen (HBsAg), anti-HBs, hepatitis Be antigen (HBeAg), anti-HBe, and antibodies to hepatitis core antigen (anti-HBc) either qualitatively or quantitatively. So high demand of the antigen to recognize the virus is necessary.

Based on a spore-based nanobody production platform, which features portability, room temperature storage, dry form, long shelf life, and high specificity, there was a proposal to develop an HVB-spore to produce the serum-based blood tests needed nanobodies which can be rapidly acquired in 24h.

### Construction of Investigative Plasmids Encoding HBV-Diagnosed Nanobodies (Nbs)

Sequences for HBV-diagnose Nbs can be obtained from the Research Collaboratory for Structural Bioinformatics Protein Data Bank (RCSB PDB) and synthesized as gene fragments after codon optimization by Twist Bioscience Inc. (San Francisco, CA, USA).

To construct recombinant plasmids containing Histag-flagged Nbs, DNA fragments will be amplified via PCR with Q5 high fidelity polymerase (NEB), and the gel-purified PCR product will be ligated into pHT43 predigested by BamHI and XbaI, through Gibson assembly. After transformation into DH5a, bacteria will be selected on LB agar plates containing 100 μg/ml ampicillin following incubation at 37° C. for 14 hr. Single colonies will be inoculated in 2 ml LB medium and grown for 12-18 hr at 37° C., plasmid will be isolated, and sent for sequence verification by Sanger Sequencing. Correct pHT43-based plasmids will be transferred into chemically prepared competent WB800N cells.

### Production and Purification of HBV-Diagnosed Nanobodies (Nbs)

Purified proteins will be obtained by growing B. subtilis WB800N containing the pHT43-based plasmid encoding the desired protein in 200 mL LB medium at 37° C. in a 2L shake flask at 250 rpm. Protein expression will be induced with 1 mM IPTG when the culture reached an OD₆₀₀ₙₘ of 0.6-0.8 and growth is continued for 18 h at 28° C. The culture supernatant will be harvested by centrifugation at 6000×g for 30 min at room temperature. Next, fresh culture supernatant containing His-tagged proteins will be purified at room temperature by gravity flow using the gravity-based column (Biorad) with cobalt agarose beads (Goldbio, St. Louis, MO, USA). The supernatant will pass through the chromatography column after the column washing with 10 column volumes of wash buffer (33 mM phosphate, 500 mM NaCl and 10 mM imidazole, pH 7.4). Protein will be eluted with elution buffer (33 mM phosphate, 150 mM NaCl, and 150 mM imidazole, pH 7.4). The OD₂₈₀ₙₘ of eluted fractions will be measured and fractions with detectable protein levels (>0.1 mg/ml) will be combined. For buffer exchange to protein storage buffer (1 mM dithiothreitol (DTT), 20 mM 4-(2-hydroxyethyl)-1-piperazineethanesulfonic acid (HEPES), 500 mM NaCl and 10% glycerol, pH 7.4), pooled supernatants will be loaded on a Amicon® ultrafiltration column. The loaded columns will be centrifuged at 4° C. and 2880×g to remove the elution buffer, diluted with storage buffer, and this process will be repeated three times. The concentrated protein solution will be transferred to a microcentrifuge tube and stored at −80° C. for further study. Purified HBV-diagnosed nanobodies will be verified by an immune test.

### INCORPORATION BY REFERENCE

All publications patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A nanobody comprising a variable domain of an antibody, wherein the nanobody is contained within a Bacillus subtilis bacteria strain.

2. The nanobody of claim 1, wherein the variable domain is a heavy chain variable domain.

3. The nanobody of claim 1 or 2, wherein the antibody is a mammalian antibody.

4. The nanobody of claim 3, wherein the mammalian antibody is a camelid antibody.

5. The nanobody of claim 1 or 2, wherein the antibody is a fish antibody.

6. The nanobody of any one of claim 1-5, wherein the Bacillus subtilis strain is deficient in at least eight extracellular proteases.

7. The nanobody of any one of claims 1-6, wherein the Bacillus subtilis strain comprises at least 90% genomic, 16S and/or CRISPR sequence identity to the nucleotide sequence of Bacillus subtilis strain WB800N (GenBank Accession No. CP032310.1).

8. The nanobody of any one of claims 1-6, wherein the Bacillus subtilis strain comprises at least 99% genomic, 16S and/or CRISPR sequence identity to the nucleotide sequence of Bacillus subtilis strain WB800N (GenBank Accession No. CP032310.1).

9. The nanobody of any one of claims 1-6, wherein the Bacillus subtilis strain is Bacillus subtilis strain WB800N (GenBank Accession No. CP032310.1).

10. The nanobody of any one of claims 1-9, further comprising an affinity tag.

11. The nanobody of claim 10, wherein the affinity tag binds to an immobile substrate.

12. The nanobody of claim 11, wherein the immobile substrate is a cellulose substrate.

13. The nanobody of any one of claims 1-12, wherein the nanobody is conjugated to a drug.

14. The nanobody of any one of claims 1-13, wherein the nanobody is conjugated to a label.

15. The nanobody of any one of claims 1-14, wherein the nanobody binds to a target antigen.

16. The nanobody of claim 15, wherein the target antigen is a small molecule.

17. The nanobody of claim 16, wherein the small molecule is methotrexate.

18. The nanobody of claim 17, wherein the nanobody comprises a nucleic acid sequence that is at least 80% identical to SEQ ID NO. 1.

19. The nanobody of claim 17, wherein the nanobody comprises a nucleic acid sequence that is at least 90% identical to SEQ ID NO. 1.

20. The nanobody of claim 17, wherein the nanobody comprises SEQ ID NO. 1.

21. The nanobody of claim 16, wherein the small molecule is caffeine.

22. The nanobody of claim 21, wherein the nanobody comprises a nucleic acid sequence that is at least 80% identical to SEQ ID NO. 2.

23. The nanobody of claim 21, wherein the nanobody comprises a nucleic acid sequence that is at least 90% identical to SEQ ID NO. 2.

24. The nanobody of claim 21, wherein the nanobody comprises SEQ ID NO. 2.

25. The nanobody of claim 15, wherein the target antigen is a eukaryotic cell surface protein.

26. The nanobody of claim 25, wherein the eukaryotic cell surface protein is an immune checkpoint ligand.

27. The nanobody of claim 26, wherein the immune checkpoint ligand is PD-L1.

28. The nanobody of claim 27, wherein the nanobody comprises an amino acid sequence that is at least 80% identical to SEQ ID NO. 3.

29. The nanobody of claim 27, wherein the nanobody comprises an amino acid sequence that is at least 90% identical to SEQ ID NO. 3.

30. The nanobody of claim 27, wherein the nanobody comprises SEQ ID NO. 3.

31. The nanobody of claim 26, wherein the immune checkpoint ligand is CTLA-4.

32. The nanobody of claim 31, wherein the nanobody comprises an amino acid sequence that is at least 80% identical to SEQ ID NO. 4.

33. The nanobody of claim 31, wherein the nanobody comprises an amino acid sequence that is at least 90% identical to SEQ ID NO. 4.

34. The nanobody of claim 31, wherein the nanobody comprises SEQ ID NO. 4.

35. The nanobody of claim 15, wherein the target antigen is a viral antigen.

36. The nanobody of claim 35, wherein the viral antigen is a SARS-CoV-2 antigen.

37. The nanobody of claim 36, wherein the SARS-CoV-2 antigen is a spike glycoprotein.

38. The nanobody of claim 35, wherein the viral antigen is a hepatitis B antigen.

39. The nanobody of claim 38, wherein the hepatitis B antigen is hepatitis B surface antigen (HBsAg).

40. The nanobody of claim 38, wherein the hepatitis B antigen is hepatitis B e-antigen (HBeAg).

41. The nanobody of claim 38, wherein the hepatitis B antigen is hepatitis B core antigen (HBcAg).

42. The nanobody of claim 15, wherein the target antigen is an inflammatory cytokine.

43. The nanobody of claim 42, wherein the inflammatory cytokine is TNF-α.

44. A cell culture supernatant of a Bacillus subtilis strain comprising a nanobody of any one of claims 1-43.

45. The cell culture supernatant of claim 44, wherein the Bacillus subtilis strain is deficient in at least eight extracellular proteases.

46. The cell culture supernatant of claim 44 or 45, wherein the Bacillus subtilis strain comprises at least 90% genomic, 16S and/or CRISPR sequence identity to the nucleotide sequence of Bacillus subtilis strain WB800N (GenBank Accession No. CP032310.1).

47. The cell culture supernatant of claim 44 or 45, wherein the Bacillus subtilis strain comprises at least 99% genomic, 16S and/or CRISPR sequence identity to the nucleotide sequence of Bacillus subtilis strain WB800N (GenBank Accession No. CP032310.1).

48. The cell culture supernatant of claim 44 or 45, wherein the Bacillus subtilis strain is Bacillus subtilis strain WB800N (GenBank Accession No. CP032310.1).

49. A vegetative cell of a Bacillus subtilis strain comprising a nanobody of any one of claims 1-43.

50. The vegetative cell of claim 49, wherein the Bacillus subtilis strain is deficient in at least eight extracellular proteases.

51. The vegetative cell of claim 49 or 50, wherein the Bacillus subtilis strain comprises at least 90% genomic, 16S and/or CRISPR sequence identity to the nucleotide sequence of Bacillus subtilis strain WB800N (GenBank Accession No. CP032310.1).

52. The vegetative cell of claim 49 or 50, wherein the Bacillus subtilis strain comprises at least 99% genomic, 16S and/or CRISPR sequence identity to the nucleotide sequence of Bacillus subtilis strain WB800N (GenBank Accession No. CP032310.1).

53. The vegetative cell of claim 49 or 50, wherein the Bacillus subtilis strain is Bacillus subtilis strain WB800N (GenBank Accession No. CP032310.1).

54. A vector comprising a polynucleotide encoding a nanobody of any one of claims 1-43.

55. A sporulated cell of a Bacillus subtilis strain comprising a vector of claim 54.

56. The sporulated cell of claim 55, wherein the Bacillus subtilis strain is deficient in at least eight extracellular proteases.

57. The sporulated cell of claim 55 or 56, wherein the Bacillus subtilis strain comprises at least 90% genomic, 16S and/or CRISPR sequence identity to the nucleotide sequence of Bacillus subtilis strain WB800N (GenBank Accession No. CP032310.1).

58. The sporulated cell of claim 55 or 56, wherein the Bacillus subtilis strain comprises at least 99% genomic, 16S and/or CRISPR sequence identity to the nucleotide sequence of Bacillus subtilis strain WB800N (GenBank Accession No. CP032310.1).

59. The sporulated cell of claim 55 or 56, wherein the Bacillus subtilis strain is Bacillus subtilis strain WB800N (GenBank Accession No. CP032310.1).

60. A pharmaceutical composition comprising a nanobody of any one of claims 1-43.

61. A pharmaceutical composition comprising a cell culture supernatant of any one of claims 44-48.

62. A pharmaceutical composition comprising a vegetative cell of any one of claims 49-53.

63. A pharmaceutical composition comprising a sporulated cell of any one of claims 55-59.

64. Use of a pharmaceutical composition of any one of claims 60-63 for the preparation of a medicament for the treatment or prevention of a disease or disorder.

65. The use of claim 64, wherein the disease or disorder is a gastrointestinal disease.

66. The use of claim 64, wherein the disease or disorder is an immunoinflammatory disease.

67. The use of claim 64, wherein the disease or disorder is a metabolic disorder.

68. The use of claim 64, wherein the disease or disorder is a cancer.

69. The use of claim 64, wherein the disease or disorder is an infectious disease.

70. The of claim 69, wherein the infectious disease is a viral infection.

71. The use of claim 70, wherein the viral infection is COVID-19.

72. The use of claim 70, wherein the viral infection is hepatitis B infection.

73. The use of any one of claims 64-72, wherein the medicament comprises an additional therapeutic.

74. The use of claim 73, wherein the additional therapeutic is an anti-inflammatory agent.

75. The use of claim 73, wherein the additional therapeutic is a chemotherapeutic agent.

76. The use of claim 73, wherein the additional therapeutic is an immunotherapy agent.

77. The use of claim 76, wherein the immunotherapy is an immune checkpoint inhibitor.

78. The use of claim 73, wherein the additional therapeutic is an anti-viral agent.

79. A method of treating a disease or disorder, comprising administering to a subject in need thereof an effective amount of the pharmaceutical composition of any one of claims 60-63.

80. The method of claim 79, wherein the disease or disorder is a gastrointestinal disease.

81. The method of claim 79, wherein the disease or disorder is a metabolic disorder.

82. The method of claim 79, wherein the disease or disorder is an immunoinflammatory disease.

83. The method of claim 79, wherein the disease or disorder is a cancer.

84. The method of claim 79, wherein the disease or disorder is an infectious disease.

85. The method of claim 84, wherein the infectious disease is a viral infection.

86. The method of claim 85, wherein the viral infection is COVID-19.

87. The method of claim 85, wherein the viral infection is hepatitis B infection.

88. The method of any one of claims 79-87, further comprising conjointly administering to the subject an effective amount of an additional therapeutic.

89. The method of claim 88, wherein the additional therapeutic is an anti-inflammatory agent.

90. The method of claim 88, wherein the additional therapeutic is a chemotherapeutic agent.

91. The method of claim 88, wherein the additional therapeutic is an immunotherapy agent.

92. The method of claim 91, wherein the immunotherapy is an immune checkpoint inhibitor.

93. The method of claim 88, wherein the additional therapeutic is an anti-viral agent.

94. A method of inhibiting virus fusion to a human cell, comprising contacting the virus with an nanobody of any one of claims 1-43.

95. The method of claim 94, wherein the virus is SARS-CoV-2 virus.

96. The method of claim 94, wherein the virus is hepatitis B virus.

97. A method of producing the nanobody of any one of claims 1-43, comprising the steps of:
a) expressing the vector of claim 54 in a Bacillus subtilis strain; and
b) harvesting the nanobody from the Bacillus subtilis strain or a cell culture supernatant of the Bacillus subtilis strain.

98. The method of claim 97, wherein the nanobody comprises an affinity tag.

99. The method of claim 98, further comprising isolating the nanobody on an immobile substrate by binding of the affinity tag to the immobile substrate.

100. The method of claim 99, wherein the immobile substrate is a cellulose substrate.

101. The method of any one of claims 97-100, wherein the Bacillus subtilis strain secretes the nanobody.

102. The method of any one of claims 97-101, wherein the Bacillus subtilis strain is deficient in at least eight extracellular proteases.

103. The method of any one of claims 97-102, wherein the Bacillus subtilis strain comprises at least 90% genomic, 16S and/or CRISPR sequence identity to the nucleotide sequence of Bacillus subtilis strain WB800N (GenBank Accession No. CP032310.1).

104. The method of any one of claims 97-102, wherein the Bacillus subtilis strain comprises at least 99% genomic, 16S and/or CRISPR sequence identity to the nucleotide sequence of Bacillus subtilis strain WB800N (GenBank Accession No. CP032310.1).

105. The method of any one of claims 97-102, wherein the Bacillus subtilis strain is Bacillus subtilis strain WB800N (GenBank Accession No. CP032310.1).

106. The method of any one of claims 101-105, wherein the nanobody is harvested from the cell culture supernatant of the Bacillus subtilis strain.

107. The method any one of claims 97-106, wherein the Bacillus subtilis strain is a sporulated Bacillus subtilis strain prior to step (a).

108. The method of claim 107, wherein the sporulated Bacillus subtilis strain is germinated into a vegetative Bacillus subtilis strain prior to step (a).

109. A method of detecting the presence of a target antigen in a sample, comprising incubating the nanobody of any one of claims 1-43 with the sample, wherein the nanobody comprises a detectable label.

110. The method of claim 109, wherein the target antigen is a small molecule.

111. The method of claim 110, wherein the small molecule is methotrexate.

112. The method of claim 110, wherein the small molecule is caffeine.

113. The method of claim 109, wherein the target antigen is a eukaryotic cell surface protein.

114. The method of claim 113, wherein the eukaryotic cell surface protein is an immune checkpoint ligand.

115. The method of claim 114, wherein the immune checkpoint ligand is PD-L1.

116. The method of claim 114, wherein the immune checkpoint ligand is CTLA-4.

117. The method of claim 109, wherein the target antigen is a viral antigen.

118. The method of claim 117, wherein the viral antigen is a SARS-CoV-2 antigen.

119. The method of claim 118, wherein the SARS-CoV-2 antigen is a spike glycoprotein.

120. The method of claim 117, wherein the viral antigen is a hepatitis B antigen.

121. The method of claim 120, wherein the hepatitis B antigen is hepatitis B surface antigen (HBsAg).

122. The method of claim 120, wherein the hepatitis B antigen is hepatitis B e-antigen (HBeAg).

123. The method of claim 120, wherein the hepatitis B antigen is hepatitis B core antigen (HBcAg).

124. The method of claim 109, wherein the target antigen is an inflammatory cytokine.

125. The method of claim 124, wherein the inflammatory cytokine is TNF-α.

126. A kit for detecting a target antigen in a sample, comprising a device for collecting the sample and reagents for detecting the target antigen, wherein the reagents comprise the nanobody of any one of claims 1-43, and the nanobody comprises a detectable label.

127. The kit of claim 126, wherein the target antigen is a small molecule.

128. The kit of claim 127, wherein the small molecule is methotrexate.

129. The kit of claim 127, wherein the small molecule is caffeine.

130. The kit of claim 126, wherein the target antigen is a eukaryotic cell surface protein.

131. The kit of claim 130, wherein the eukaryotic cell surface protein is an immune checkpoint ligand.

132. The kit of claim 131, wherein the immune checkpoint ligand is PD-L1.

133. The kit of claim 131, wherein the immune checkpoint ligand is CTLA-4.

134. The kit of claim 126, wherein the target antigen is a viral antigen.

135. The kit of claim 134, wherein the viral antigen is a SARS-CoV-2 antigen.

136. The kit of claim 135, wherein the SARS-CoV-2 antigen is a spike glycoprotein.

137. The kit of claim 134, wherein the viral antigen is a hepatitis B antigen.

138. The kit of claim 137, wherein the hepatitis B antigen is hepatitis B surface antigen (HBsAg).

139. The kit of claim 137, wherein the hepatitis B antigen is hepatitis B e-antigen (HBeAg).

140. The kit of claim 137, wherein the hepatitis B antigen is hepatitis B core antigen (HBcAg).

141. The kit of claim 126, wherein the target antigen is an inflammatory cytokine.

142. The kit of claim 141, wherein the inflammatory cytokine is TNF-α.
